(19) United States
(12) Patent Application Publication
(10) Pub. No.: US 2011/0059485 A1
(43) Pub. Date: Mar. 10, 2011

(54) Plasmids from Thermophilic Organisms, Vectors Derived Therefrom, and Uses Thereof

(75) Inventors: Nicky Caiazza, Hanover, NH (US); Anne Warner, Lebanon, NH (US); Chris Herring, Lebanon, NH (US)

(73) Assignee: Mascoma Corporation, Lebanon, NH (US)

(21) Appl. No.: 12/677,428

(22) PCT Filed: Sep. 10, 2008
(86) PCT No.: PCT/US08/10545

§ 371 Date: Nov. 12, 2010

Related U.S. Application Data
(60) Provisional application No. 60/971,225, filed on Sep. 10, 2007.

(51) Int. Cl. C12P 21/02; C07H 21/04; C12N 15/63; C12N 15/70; C12N 15/74; C12N 1/21; C12N 1/19; C12P 19/34; C12N 15/64; C07K 14/00; C12N 9/16

(52) U.S. Cl. 435/69.1; 536/23.7; 435/320.1; 435/252.3; 435/255.1; 435/91.1; 435/91.4; 530/350; 435/196

(57) ABSTRACT

The present invention is directed to a replicative, thermostable plasmid. In particular, the present invention is directed to a replicative, thermostable plasmid comprising a sequence derived from the pB6A plasmid and at least one functional unit comprising a sequence that is not found in plasmid pB6A.

### BACKGROUND OF THE INVENTION

1. Field of the Invention

The present invention relates to the field of molecular biology, and in particular, to thermophilic organisms and plasmids that are stably maintained in such organisms.

2. Background Art

Thermophilic microorganisms, which can grow at temperatures of 45° C. and above, are useful for a variety of industrial processes. For example, thermophilic microorganisms can be used as biocatalysts in reactions at higher operating temperatures than can be achieved with mesophilic microorganisms. Thermophilic organisms are particularly useful in biologically mediated processes for energy conversion, such as the production of ethanol from plant biomass, because higher operating temperatures allow more convenient and efficient removal of ethanol in vaporized form from the fermentation medium.

The ability to metabolically engineer thermophilic microorganisms to improve various properties (e.g., ethanol production, breakdown of lignocellulosic materials), would allow the benefit of higher operating temperatures to be combined with the benefits of using industrially important enzymes from a variety of sources in order to improve efficiency and lower the cost of production of various industrial processes, such as energy conversion and alternative fuel production. Important tools for genetically engineering thermophilic microorganisms are plasmids that can survive and self-replicate in thermophilic hosts.

To date, very few plasmids have been identified from thermophilic microorganisms, considering the number of thermophilic hosts that have been characterized, and plasmids that are stable in thermophilic hosts such as Thermoanaerobacterium saccharolyticum, Clostridium thermocellum, have not been usefully characterized. Weimer et al., Arch. Microbiol. (1984) 138:31-36, identified plasmids in four out of seven thermophilic anaerobic bacteria (including the B6A strain), but did no more than determine the size of the plasmids on an agarose gel. Ahring et al. U.S. Pat. Appl. Publ. No. 2005/0026293 A1, isolated and characterized three plasmids from Anaerocellum thermophilum DSM6725 for use as vectors, but did not characterize plasmids from T. saccharolyticum or other thermophilic bacteria.

In certain cases, the current suite of vectors available for use in thermophilic hosts can be used to deliver DNA into the host cell and, through subsequent recombination events, plasmid-associated markers can be selected for after chromosomal integration. This has been demonstrated for T. saccharolyticum, for example, but not C. thermocellum. This use of a plasmid is suitable for disrupting genes and placing foreign DNA into the host chromosome in a directed fashion. However, many plasmid uses require that the plasmid be stable and capable of autonomous replication. For instance, the ability to establish reporters, expression systems, and complementation studies are greatly facilitated with stable plasmids. Furthermore, the use of an autonomously-replicating, thermostable plasmid would be valuable for use as a shuttle vector and for expression of exogenous enzymes and proteins in industrial processes. However, not all replication proteins from thermophilic bacteria can be used to create shuttle vectors between thermophilic and mesophilic hosts. For example, Belogurova et al., Mol. Biol. (2002) 36: 106-113, demonstrated that expression of the replication protein RepN encoded by the RC plasmid of T. saccharolyticum was lethal in E. coli.

Therefore, there remains a need for replicative plasmids that are stable at the temperatures of thermophilic hosts, e.g., at about 45° C. and above. Likewise, there is a need for replicative, thermostable plasmids that can serve a variety of purposes, such as a shuttle vector between different hosts (including both thermophilic and non-thermophilic hosts), a cloning vector, an expression vector, and a reporter system.

### BRIEF SUMMARY OF THE INVENTION

In one aspect, the present invention is generally directed to a plasmid derived from Thermoanaerobacterium saccharolyticum strain B6A that is thermostable and can autonomously replicate in thermophilic hosts. In another aspect the present invention is directed to replicative, thermostable plasmids for use as cloning vectors, shuttle vectors, expression vectors, and reporter systems.

In a further aspect, the present invention is directed to an isolated plasmid comprising a nucleotide sequence encoding a polypeptide having Rep protein activity, wherein the polypeptide is at least 90% identical to the amino acid sequence of SEQ ID NO:22. In a preferred embodiment, the plasmid is stable and replicative in a thermophilic host.

In a further aspect, the present invention is directed to an isolated plasmid comprising a nucleotide sequence encoding a polypeptide having Rep protein activity, wherein the polypeptide is at least 90% identical to the amino acid sequence of SEQ ID NO:22; and at least one functional unit comprising a nucleotide sequence that is not found in plasmid pB6A (SEQ ID NO:9) or the plasmid isolated from the T. Saccharolyticum type strain B6A deposited as ATCC No. 49915. In one embodiment, the plasmid is replicative and stable in a thermophilic host. In one embodiments, the functional unit is selected from the group consisting of a replicon, an origin of replication, a sequence encoding a protein or a functional protein fragment, a restriction site, a multiple cloning site, and any combination thereof.

In another aspect, the invention is directed to an isolated nucleic acid comprising a sequence that is at least about 90%, 95%, 96%, 97%, 98%, 99%, or 100% identical to SEQ ID NO:21, wherein said nucleic acid does not consist only of the plasmid pB6A of SEQ ID NO:9 or the plasmid isolated from T. Saccharolyticum type strain B6A deposited as ATCC No. 49915. In a further aspect, the invention is directed to an isolated nucleic acid comprising a sequence that encodes a polypeptide that is at least about 90%, 95%, 96%, 97%, 98%, 99%, or 100% identical to the amino acid sequence of SEQ ID NO:22, wherein said nucleic acid does not consist only of the plasmid pB6A of SEQ ID NO:9 or the plasmid isolated from T. Saccharolyticum type strain B6A deposited as ATCC No. 49915. In a further aspect, the invention is directed to a plasmid comprising the isolated nucleic acids, wherein the plasmid does not consist only of the plasmid pB6A of SEQ ID NO:9 or the plasmid isolated from T. Saccharolyticum type strain B6A deposited as ATCC No. 49915.

In another aspect, the isolated plasmid comprises a gram-positive rolling circle origin of replication. In a particular aspect the origin of replication comprises SEQ ID NO:30.

In another aspect, the functional unit is a replicon, preferably a broad host-range replicon. In another aspect, the broad host range replicon is selected from the group consisting of: an RK2 replicon, a pRO1600 replicon, and a p15a/ColE1 replicon. In another aspect, the replicon is functional in one or more organisms selected from Acetobacter, Achromobacter, Acinetobacter, Aeromonas, Agrobacterium, Alcaligenes, Anabaena, Azospirrillum, Azotobacter, Bartonella, Bordetella, Caulobacter, Clavobacter, Enterobacteriaceae, Haemophilus, Hypomycrobium, Legionella, Klebsiella, Methylophilus, Methylosinus, Myxococcus, Neisseria, Paracoccus, Proteus, Pseudomonas, Rhizobium, Rhodopseudomonas, Rhodospirillum, Salmonella, Serratia, Thiobacillus, Vibrio, Xanthomonas, Yersinia, and Zymomonas. In certain aspects, the replicon that is functional in one or more organisms is a second replicon within a plasmid or shuttle vector.

In another aspect, the functional unit is a yeast replicon. In further aspects, the yeast replicon is CEN6/ARSH.

In another aspect, the functional unit encodes a selectable marker. In a further aspect, the selectable marker is resistance to an antibiotic selected from ampicillin, kanamycin, erythromycin, chloramphenicol, gentamycin, kasugamycin, rifampicin, spectinomycin, D-Cycloserine, nalidixic acid, streptomycin, tetracycline, or combinations thereof.

In another aspect, the selectable marker is a nutritional marker.

In another aspect; the selectable marker is a yeast selectable marker. In further aspects the yeast selectable marker is selected from the group consisting of URA3, HIS3, LEU2, TRP1, LYS2 and ADE2.

In another aspect, the functional unit is a multiple cloning site. In a further aspect, the multiple cloning site comprises one or more restriction sites selected from HindIII, MluI, SpelI BglII, StuI, BspDI/ClaI, PvuII, NdeI, NcoI, SmaI/XmaI, PvuI, EagI/XmaIII, PaeR7I/XhoI, PstI, EcoRI, SqacI, EcoRV, SphI, NaeI, NheI, BamHI, NarI, ApaI, Acc65I/KpnI, SalI, ApaLI, HpaI, BspEI, NruI, XbaI, BclI, BalI, SwaI, Sse8387I, SrfI, NotI, AscI, PacI, and PmeI, or combinations thereof.

In another aspect, the functional unit comprises a sequence that encodes a protein or functional protein fragment. In a further aspect, the protein or functional fragment thereof facilitates the anaerobic oxidation of an organic compound. In a further aspect, the protein or functional protein fragment is an enzyme. In a further aspect, the enzyme is a saccharolytic enzyme or a fermentation enzyme.

In another aspect, the functional unit comprises a sequence that encodes a reporter gene. In one aspect, the reporter gene encodes a protein that is functional in anaerobic conditions. In a further aspect, the reporter gene is catechol 2,3-oxygenase (xylE). In a further aspect, the reporter gene is selected from the group consisting of: β-galactosidase, β-glucuronidase, luciferase, green fluorescent protein, red fluorescent protein or combinations thereof. In a still further aspect, the reporter gene further comprises a promoter. In a still further aspect, the promoter is a heterologous promoter.

In another aspect, the plasmid comprises the sequence of SEQ ID NO:10 or the sequence of the plasmid deposited at the ATCC as ______.

In another aspect, the plasmid comprises the sequence of SEQ ID NO:11 or the sequence of the plasmid deposited at the ATCC as ______.

In another aspect, the plasmid comprises the sequence of SEQ ID NO:14.

In another aspect, the plasmid comprises the sequence of SEQ ID NO:17.

In another aspect, the plasmid comprises the sequence of SEQ ID NO:20.

In another aspect, the plasmid comprises the sequence of SEQ ID NO:25.

In another aspect, the plasmid comprises the sequence of SEQ ID NO:28.

In another aspect, the plasmid comprises the sequence of SEQ ID NO:39.

In another aspect, the plasmid comprises the sequence of SEQ ID NO:40.

In another aspect, the plasmid of the present invention is a shuttle vector. In further aspects, the shuttle vector is an E. coli-S. cerevisiae-thermophile shuttle vector. In additional embodiments, the E. coli-S. cerevisiae-thermophile shuttle vector comprises a gram-positive rolling circle origin of replication, an antibiotic-resistance gene, a yeast selectable marker, and a yeast replicon.

In another aspect, the E. coli-S. cerevisiae-thermophile shuttle vector comprises a selectable marker for a thermophilic bacterium.

In another aspect, the invention is directed to a host cell comprising an isolated plasmid of the present invention. In a further aspect, the host cell is a bacterium.

In a further aspect, the bacterium is a thermophilic bacterium selected from one or more of a Thermoanaerobacterium species, Clostridium species, Thermoanaerobacter species, Thermobacteroides species, Anaerocellum species, and Caldicellulosiruptor species.

In another aspect, the host cell is a yeast cell. In a further aspect, the yeast cell is a thermophilic yeast cell.

In another aspect, the present invention is directed to a method for expressing a heterologous sequence in a thermophilic host, comprising transforming a thermophilic host with a plasmid of the present invention; and culturing the transformed thermophilic host for a length of time and under conditions whereby the heterologous sequence is expressed.

In another aspect, the present invention is directed to a method of producing a replicative, thermostable plasmid, comprising obtaining a nucleotide sequence encoding a polypeptide having Rep protein activity, wherein the polypeptide is at least 90% identical to the amino acid sequence of SEQ ID NO:22, or a functional fragment thereof; obtaining at least one functional unit comprising a sequence that is not found in plasmid pB6A (SEQ ID NO:9) or the plasmid isolated from T. Saccharolyticum type strain B6A deposited as ATCC No. 49915.; and combining the nucleotide sequences together.

In another aspect, the present invention is directed to a method of producing a shuttle vector, comprising providing a first replicon that is autonomously replicable in a first host, wherein the replicon comprises a nucleotide sequence encoding a polypeptide having Rep protein activity, wherein the polypeptide is at least 90% identical to the amino acid sequence of SEQ ID NO:22, or a functional fragment thereof; obtaining a fragment of the first replicon comprising at least the nucleotide sequence encoding a polypeptide having Rep protein activity by utilizing routine molecular biology techniques known in the art, such as restriction enzyme digestion, polymerase chain reaction (PCR) or oligonucleotide synthesis; providing a second replicon that is heterologous to the first replicon and autonomously replicable in a second host and obtaining a fragment of the second replicon comprising at least an origin of replication using routine molecular biology techniques known in the art, as described above; and ligating, fusing, or assembling together the fragment of the first replicon with the fragment of the second replicon to obtain a shuttle vector that is autonomously replicable in both the first host and the second host. In another embodiment, the method further comprises providing a third replicon that is heterologous to the first and second replicons, and that is autonomously replicable in a third host, with one or more restriction enzymes to obtain a fragment of the third replicon comprising at least an origin of replication; and ligating and/or assembling the fragments of the first, second, and third replicons to obtain a shuttle vector that is autonomously replicable in the first, second, and third hosts. In another aspect, the invention is directed to a shuttle vector produced by these methods.

In another aspect, the invention is directed to an isolated polypeptide comprising a sequence that is at least about 90%, 95%, 96%, 97%, 98%, 99% or 100% identical to SEQ ID NO:22 or a functional fragment thereof. In one embodiment, the functional fragment has DNA nicking activity. In another embodiment, the functional fragment has specific origin site recognition activity.

### BRIEF DESCRIPTION OF THE FIGURES

FIG. 1A. Isolation of pMU120 (pB6A) from Thermoanaerobacterium saccharolyticum strain B6A. The left lane of the gel (“ladder”) shows the supercoiled DNA ladder. The right lane (“pB6A”) shows a strong band at approximately 2,300 base pairs, which represents the supercoiled DNA, and a faint band at approximately 4,500 base pairs, which represents slower-moving nicked or relaxed DNA.

FIG. 1B. Gel purification of a 2,300 base pair band from the gel in FIG. 1A. The left lane of the gel (“ladder”) shows the supercoiled DNA ladder. The right lane (“pB6A”), again shows a strong band at approximately 2,300 base pairs, which represents the supercoiled DNA, and a faint band at approximately 4,500 base pairs, which represents slower-moving nicked or relaxed DNA.

FIG. 2. Putative clones containing fragments of pMU120 restriction digestion with AseI. Fragments generated by digestion with AseI were cloned into pUC19 and digested with XmnI and EcoRI. Lanes 1-5 represent fragments from the digestion of pUC19 which contain AseI-generated fragments of pMU120. Lane 6 represents the same digest performed on a control pUC19 vector with no inserts. Lane 7 represents the digest of plasmid pMU120 with AseI.

FIG. 3. Map of assembly of fragments of pMU120. Inserts from the AseI digest were used to design sequencing primers to sequence additional regions of pMU120. The sequenced fragments were assembled based on their overlap.

FIG. 4. Map of pMU120 (pB6A). The map shows the location of primers used in the sequencing reactions. Primer X00254 is represented by SEQ ID NO:3; Primer X00255 is represented by SEQ ID NO:4; Primer X00256 is represented by SEQ ID NO:5; Primer X00316 is represented by SEQ ID NO:7. The location of the MfeI restriction site is also shown. The sequence of pMU120 is shown in SEQ ID NO:9.

FIG. 5. Open reading frame map of pMU120 (pB6A). The map shows the location of primers used in the sequencing reactions and putative open reading frames (slender arrows). The thick arrow represents an open reading frame that shares homology with the repB gene of cryptic plasmid pCB101 found in Clostridium butyricum. The location of the MfeI restriction site is also shown.

FIG. 6A-B. Maps of plasmid pMU121 (pB6ApUC). Panels A and B both represent maps of pMU121, showing the result of ligating pMU120 into the EcoRI site of pUC19. Plasmid pMU121 (SEQ ID NO:10) contains a selective marker for ampicillin resistance (AP^{r}), shown in both panels A and B. Panel A shows the multiple cloning site of pMU121, the ApaLI restriction sites, and the locations of the sequences that correspond to primers X00254, X00255, X00256, and X00316. Panel B shows the location of the sequence encoding repB in pMU121, as well as the SapI site.

FIG. 7. Map of plasmid pMU131. A HindIII restriction digest fragment containing the kanamycin resistance gene (“Kn”) and a suspected promoter from plasmid pIKM1 was ligated into pMU121 to create pMU131 (SEQ ID NO:11).

FIG. 8. Confirmation of transformation of T. saccharolyticum by pMU131. Lane 1 of the gel represents a 1 kb DNA ladder (New England Biolabs® Inc.). Lane 4 represents plasmid pMU131 digested with BamH1. Lanes 2 and 3 represent plasmid DNA recovered from the transformed T. saccharolyticum hosts and digested with BamHI. The candidate plasmids in lanes 2 and 3 run at approximately 6.4 kb, the size expected for pMU131.

FIG. 9. Map of plasmid pMU141. Chloramphenicol resistance (“CM(R)”) and erythromycin resistance (“ERY(R)”) genes were amplified from pJIR418 and engineered with HindIII sites for ligation into pMU121 to create pMU141 (pB6ApUCcatery) (SEQ ID NO:14).

FIG. 10. Map of plasmid pMU144. The chloramphenicol resistance (“CM(R)”) gene was amplified from pJIR418 and engineered with HindIII sites for ligation into pMU121 to create pMU141 (pB6ApUCcat) (SEQ ID NO:20).

FIG. 11. Map of plasmid pMU143. The erythromycin resistance (“ERY(R)”) gene was amplified from pJIR418 and engineered with HindIII sites for ligation into pMU121 to create pMU143 (pB6ApUCery) (SEQ ID NO:17).

FIG. 12. Map of plasmid pMU110. The pMU110 plasmid was used to obtain the Ura3-Cen6/Arsh region by PCR amplification. Location of the PCR primers X00592 and X00593 are indicated.

FIG. 13. Map of plasmid pMU158. This map shows the result of ligating SapI-linearized pMU121 with a yeast Ura3-Cen6/Arsh selectable marker. Plasmid pMU158 (SEQ ID NO:25) also contains a selective marker for ampicillin resistance (AP^{r}), an origin of replication, and the repB sequence described herein.

FIGS. 14A-D. Construction of the pMU158 plasmid. A. Linearization of pMU121 with Sap I. Lane 1 shows an NEB 1 kb ladder. The fourth band from the top in the ladder lane corresponds to 5 kb. Lane 2 shows the predicted approximately 5 kb DNA fragment corresponding to pMU121 digested with Sap I. B. Amplified Ura3-Cen6/Arsh. Primers X00592 and X00593 were used to amplify the Ura3-Cen6/Arsh region of pMU110 and clone this fragment into pMU121 using yeast mediated ligation. Lane 1 shows a 1 kb ladder (the second band from the bottom corresponds to 1.5 kb). Lane 2 shows the amplified Ura3-Cen6/Arsh migrating at approximately 1.7 kb. C. Restriction enzyme analysis of pMU158 with BamH1 and NcoI. Lane 1 shows the DNA ladder. The fourth band from the top is 5 kb and the bottom band is 1 kb. Lanes 2-4 show the expected 5.4 and 1.2 kb bands generated from the BamHI/NcoI double digest. D. Restriction enzyme analysis of pMU158 with BglII. Lane 1 shows the DNA ladder. The fourth band from the top is 5 kb and the bottom band is 1 kb. Lanes 2-4 show the predicted 4.9 and 1.6 kb bands generated from the BglII digest.

FIG. 15. Map of pMU105. The pMU105 plasmid was used to obtain the kanamycin resistance (“Kn”) gene by PCR amplification. Location of the PCR primers X00613 and X00615 are indicated.

FIG. 16. The kanamycin resistance gene (“Kn”) generated by PCR amplification. Lane 1 shows the NEB DNA ladder. The third band from the bottom in the ladder lane is 1.5 kb. Lane 2 shows the amplified product running at the expected size of 1,475 bp.

FIG. 17. Map of pMU166. This map shows the result of ligating pMU158 with an amplicon containing the E. Coli selective marker for kanamycin (Kn). The pMU166 (SEQ ID NO:28) plasmid also contains a yeast origin of replication, a yeast Ura3-Cen6/Arsh selectable marker, and the repB sequence.

FIG. 18. Digestion of pMU166 with EcoRV. Lane 1 corresponds to the DNA ladder. The bottom four bands are 3.0, 2.0, 1.5. and 1.0 kb, respectively. Lanes 2-4 show DNA fragments generated from the digestion of three independent isolates of the pMU166 plasmid with EcoRV.

FIG. 19. Comparison of Ura3 expression between T. Saccharolyticum harboring pMU675 plasmid and Ura3+ T. Saccharolyticum strain ALK2. Expression from pMU675 was greater than 10,000-fold higher.

FIG. 20. Map of pMU675. This map shows plasmid pMU675 (SEQ ID NO:39) constructed by fusing and inserting PCR-amplified kanamycin selectable marker, the C. thermocellum CBP promoter, the T. Saccharolyticum Ura3 gene, and the T1+T2 terminator sequence into the pMU158 backbone (SEQ ID NO:25) using yeast-mediated ligation.

FIG. 21A-B. A) PCR screen of catD insert for pMU362. Positive band at 1253 bp indicates that all 7 clones screened were positive. B) Clones #2 and #3 were further screened using a BamHI+EcoRV digest (lanes 1 and 3) with expected bands at 3.7, 1.5, 1.1 Kb, 363 bp and an ApalI+SacI (lanes 2 and 4) digest with expected bands at 3.3, 2.5, 1.2, and 0.5 kb.

FIG. 22. Gel analysis of the EcoRV+SacI digest of T. Saccharolyticum pMU362 plasmid isolation. All eight colonies indicate the presence of the pMU362 plasmid as compared to the lane 10 pMU362 control. Lane 11 is the pMU131 digest control.

FIG. 23. Map of pMU362. This map shows the construction of pMU362 (SEQ ID NO:40) by cloning the catD chloramphenicol resistance gene and its native promoter into the pCR2.1-TOPO TA cloning vector (Invitrogen). The fragment was gel purified from the TOPO vector and ligated into the pMU131 vector (SEQ ID NO:11) using the BamHI and PstI restriction sites.

### DETAILED DESCRIPTION OF THE INVENTION

The present invention relates to, inter alfa, the isolation, construction, and use of thermostable plasmids. Applicants have isolated and characterized a thermostable plasmid, pB6A (also referred to herein as pMU120), from Thermoanaerobacterium saccharolyticum strain B6A and constructed novel Escherichia coli-thermophile shuttle vectors using pB6A (e.g., pMU121 (SEQ ID NO:10), pMU131 (SEQ ID NO:11), pMU141 (SEQ ID NO:14), pMU143 (SEQ ID NO:17), pMU144 (SEQ ID NO:20), pMU158 (SEQ ID NO:25), pMU166 (SEQ ID NO:28), pMU675 (SEQ ID NO:39), and pMU362 (SEQ ID NO:40)). Applicants' invention provides important tools for use in genetically engineering thermophilic microorganisms. In addition, Applicants have identified a unique replication protein, repB (SEQ ID NOs:21 and 22), from the plasmid pMU120. This replication protein-encoding nucleic acid (and its expression product) may be used in a variety of cloning and expression vectors and, particularly, in shuttle vectors for the expression of homologous and heterologous genes in thermophilic microorganisms such as bacteria and yeast.

### Definitions

A “plasmid” or “vector” refers to an extrachromosomal element often carrying one or more genes that are not part of the central metabolism of the cell, and is usually in the form of a circular double-stranded DNA molecule. Such elements may be autonomously replicating sequences, genome integrating sequences, phage or nucleotide sequences, linear, circular, or supercoiled, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. Preferably, the plasmids or vectors of the present invention are stable and self-replicating.

An “expression vector” is a vector that is capable of directing the expression of genes to which it is operably linked.

A “shuttle vector” is a cloning vector that is capable of replication and/or expression in more than one host cell type.

The term “thermophilic” refers to an organism that grows and thrives at a temperature of about 45° C. or higher.

The term “mesophilic” refers to an organism that grows and thrives at a temperature of about 25° C. to about 40° C.

A “replicon” is a genetic element that behaves as an autonomous unit during DNA replication. In a non-limiting example, the replicon is a broad host range replicon (a recognized term of art), such as an RK2 replicon, a pRO1600 replicon, or a p15a/ColE1 replicon. In a non-limiting example, the replicon is functional in an organism selected from the genera consisting of: Acetobacter, Achromobacter, Acinetobacter, Aeromonas, Agrobacterium, Alcaligenes, Anabaena, Anaerocellum, Azospirrillum, Azotobacter, Bartonella, Bordetella, Caldicellulosiruptor, Caulobacter, Clavobacter, Clostridium, Enterobacteriaceae, Haemophilus, Hypomycrobium, Legionella, Klebsiella, Methylophilus, Methylosinus, Myxococcus, Neisseria, Paracoccus, Proteus, Pseudomonas, Rhizobium, Rhodopseudomonas, Rhodospirillum, Salmonella, Serratia, Thermoanaerobacter, Thermoanaerobacterium, Thermobacteroides, Thiobacillus, Vibrio, Xanthomonas, Yersinia, and Zymomonas.

A “selectable marker” is a gene, the expression of which creates a detectable phenotype and which facilitates detection of host cells that contain a plasmid having the selectable marker. Non-limiting examples of selectable markers include drug resistance genes and nutritional markers. For example, the selectable marker can be a gene that confers resistance to an antibiotic selected from the group consisting of: ampicillin, kanamycin, erythromycin, chloramphenicol, gentamycin, kasugamycin, rifampicin, spectinomycin, D-Cycloserine, nalidixic acid, streptomycin, or tetracycline. Other non-limiting examples of selection markers include adenosine deaminase, aminoglycoside phosphotransferase, dihydrofolate reductase, hygromycin-B-phosphotransferase, thymidine kinase, and xanthine-guanine phosphoribosyltransferase. A single plasmid can comprise one or more selectable markers.

The term “heterologous” as used herein refers to an element of a plasmid or cell that is derived from a source other than the endogenous source. Thus, for example, a heterologous sequence could be a sequence that is derived from a different gene or plasmid from the same host, from a different strain of host cell, or from an organism of a different taxonomic group (e.g., different kingdom, phylum, class, order, family genus, or species, or any subgroup within one of these classifications). The term “heterologous” is also used synonymously herein with the term “exogenous.”

The term “functional unit” as used herein refers to any sequence which represents a structural or regulatory feature, region, or element. Such functional units, include, but are not limited to a replicon, an origin of replication, a sequence encoding a protein or a functional protein fragment, a restriction site, a multiple cloning site, and any combination thereof. The functional unit may be an untranslated nucleic acid sequence (for example, with regulatory properties or functions) or a sequence for a gene encoding a protein (for example, a structural or regulatory gene).

The term “stable plasmid” refers to a plasmid that is capable of autonomous replication and which is maintained throughout at least one and preferably many successive generations of host cell division. A “thermostable plasmid” is a plasmid that is stable at the temperatures of a thermophilic host.

A “reporter gene” is a gene that produces a detectable product that is connected to a promoter of interest so that detection of the reporter gene product can be used to evaluate promoter function. A reporter gene may also be fused to a gene of interest (e.g., 3′ to the endogenous promoter of the gene of interest), such that the fused genes are expressed as a fusion protein that allow one to detect whether the gene of interest is expressed under a given set of conditions. Non-limiting examples of reporter genes include: β-galactosidase, β-glucuronidase, luciferase, chloramphenicol acetyltransferase (CAT), secreted alkaline phosphatase (SEAP), green fluorescent protein (GFP), red fluorescent protein (RFP), and catechol 2,3-oxygenase (xylE).

A “nucleic acid” is a polymeric compound comprised of covalently linked subunits called nucleotides. Nucleic acid includes polyribonucleic acid (RNA) and polydeoxyribonucleic acid (DNA), both of which may be single-stranded or double-stranded. DNA includes cDNA, genomic DNA, synthetic DNA, and semi-synthetic DNA.

An “isolated nucleic acid molecule” or “isolated nucleic acid fragment” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester anologs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5′ to 3′ direction along the non-transcribed strand of DNA (i.e., the strand having a sequence homologous to the mRNA).

A “gene” refers to an assembly of nucleotides that encode a polypeptide, and includes cDNA and genomic DNA nucleic acids. “Gene” also refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences.

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength. Hybridization and washing conditions are well known and exemplified in Sambrook, J., Fritsch, E. F. and Maniatis, T. MOLECULAR CLONING: A LABORATORY MANUAL, Second Edition, Cold Spring Harbor Laboratory Press, Cold Spring Harbor (1989), particularly Chapter 11 and Table 11.1 therein (hereinafter “Maniatis”, entirely incorporated herein by reference). The conditions of temperature and ionic strength determine the “stringency” of the hybridization. Stringency conditions can be adjusted to screen for moderately similar fragments, such as homologous sequences from distantly related organisms, to highly similar fragments, such as genes that duplicate functional enzymes from closely related organisms. Post-hybridization washes determine stringency conditions. One set of preferred conditions uses a series of washes starting with 6× SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 min. A more preferred set of stringent conditions uses higher temperatures in which the washes are identical to those above except for the temperature of the final two 30 min washes in 0.2×SSC, 0.5% SDS was increased to 60° C. Another preferred set of highly stringent conditions uses two final washes in 0.1×SSC, 0.1% SDS at 65° C. Another set of highly stringent conditions are defined by hybridization at 0.1×SSC, 0.1% SDS, 65° C. and washed with 2×SSC, 0.1% SDS followed by 0.1×SSC, 0.1% SDS.

Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tm for hybrids of nucleic acids having those sequences. The relative stability (corresponding to higher Tm) of nucleic acid hybridizations decreases in the following order: RNA:RNA, DNA:RNA, DNA:DNA. For hybrids of greater than 100 nucleotides in length, equations for calculating Tm have been derived (see, e.g., Maniatis at 9.50-9.51). For hybridizations with shorter nucleic acids, i.e., oligonucleotides, the position of mismatches becomes more important, and the length of the oligonucleotide determines its specificity (see, e.g., Maniatis, at 11.7-11.8). In one embodiment the length for a hybridizable nucleic acid is at least about 10 nucleotides. Preferably a minimum length for a hybridizable nucleic acid is at least about 15 nucleotides; more preferably at least about 20 nucleotides; and most preferably the length is at least 30 nucleotides. Furthermore, the skilled artisan will recognize that the temperature and wash solution salt concentration may be adjusted as necessary according to factors such as length of the probe.

The term “percent identity”, as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those described in: Computational Molecular Biology (Lesk, A. M., ed.) Oxford University Press, NY (1988); Biocomputing: Informatics and Genome Projects (Smith, D. W., ed.) Academic Press, NY (1993); Computer Analysis of Sequence Data, Part I (Griffin, A. M., and Griffin, H. G., eds.) Humana Press, NJ (1994); Sequence Analysis in Molecular Biology (von Heinje, G., ed.) Academic Press (1987); and Sequence Analysis Primer (Gribskov, M. and Devereux, J., eds.) Stockton Press, NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignment of the sequences was performed using the Clustal method of alignment (Higgins and Sharp (1989) CABIOS. 5:151-153) with the default parameters (GAP PENALTY=10, GAP LENGTH PENALTY=10). Default parameters for pairwise alignments using the Clustal method were KTUPLE 1, GAP PENALTY=3, WINDOW=5 and DIAGONALS SAVED=5.

Suitable nucleic acid sequences or fragments thereof (including any of the isolated polynucleotides of the present invention) encode polypeptides that are at least about 70% to 75% identical to the amino acid sequences reported herein, preferably at least about 80%, 85%, or 90% identical to the amino acid sequences reported herein, and most preferably at least about 95%, 96%, 97%, 98%, 99%, or 100% identical to the amino acid sequences reported herein. Suitable nucleic acid fragments are preferably at least about 70%, 75%, or 80% identical to the nucleic acid sequences reported herein, preferably at least about 80%, 85%, or 90% identical to the nucleic acid sequences reported herein, and most preferably at least about 95%, 96%, 97%, 98%, 99%, or 100% identical to the nucleic acid sequences reported herein. Suitable nucleic acid fragments not only have the above identities/similarities but typically encode a polypeptide having at least 50 amino acids, preferably at least 100 amino acids, more preferably at least 150 amino acids, still more preferably at least 200 amino acids, and most preferably at least 250 amino acids.

The term “probe” refers to a single-stranded nucleic acid molecule that can base pair with a complementary single stranded target nucleic acid to form a double-stranded molecule.

The term “complementary” is used to describe the relationship between nucleotide bases that are capable to hybridizing to one another. For example, with respect to DNA, adenosine is complementary to thymine and cytosine is complementary to guanine. Accordingly, the instant invention also includes isolated nucleic acid fragments that are complementary to the complete sequences as reported in the accompanying Sequence Listing as well as those substantially similar nucleic acid sequences.

As used herein, the term “oligonucleotide” refers to a nucleic acid, generally of about 18 nucleotides, that is hybridizable to a genomic DNA molecule, a cDNA molecule, or an mRNA molecule. Oligonucleotides can be labeled, e.g., with ³²P-nucleotides or nucleotides to which a label, such as biotin, has been covalently conjugated. An oligonucleotide can be used as a probe to detect the presence of a nucleic acid according to the invention. Similarly, oligonucleotides (one or both of which may be labeled) can be used as PCR primers, either for cloning full length or a fragment of a nucleic acid of the invention, or to detect the presence of nucleic acids according to the invention. Generally, oligonucleotides are prepared synthetically, preferably on a nucleic acid synthesizer. Accordingly, oligonucleotides can be prepared with non-naturally occurring phosphoester analog bonds, such as thioester bonds, etc.

A DNA “coding sequence” is a double-stranded DNA sequence which is transcribed and translated into a polypeptide in a cell in vitro or in vivo when placed under the control of appropriate regulatory sequences. “Suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, RNA processing site, effector binding site and stem-loop structure. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from mRNA, genomic DNA sequences, and even synthetic DNA sequences. If the coding sequence is intended for expression in a eukaryotic cell, a polyadenylation signal and transcription termination sequence will usually be located 3′ to the coding sequence.

“Open reading frame” is abbreviated ORF and means a length of nucleic acid sequence, either DNA, cDNA or RNA, that comprises a translation start signal or initiation codon, such as an ATG or AUG, and a termination codon and can be potentially translated into a polypeptide sequence.

“Promoter” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

A coding sequence is “under the control” of transcriptional and translational control sequences in a cell when RNA polymerase transcribes the coding sequence into mRNA, which is then trans-RNA spliced (if the coding sequence contains introns) and translated into the protein encoded by the coding sequence.

“Transcriptional and translational control sequences” are DNA regulatory sequences, such as promoters, enhancers, terminators, and the like, that provide for the expression of a coding sequence in a host cell. In eukaryotic cells, polyadenylation signals are control sequences.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The term “expression,” as used herein, refers to the transcription and stable accumulation of sense (mRNA) or antisense RNA derived from the nucleic acid fragment of the invention. Expression may also refer to translation of mRNA into a polypeptide.

The terms “restriction endonuclease” and “restriction enzyme” refer to an enzyme which binds and cuts at a specific nucleotide sequence within double stranded DNA.

A “derivative” of the plasmid of the present invention means a plasmid comprising a part of the plasmid of the present invention, or the plasmid of present invention and another DNA sequence. The “part of a plasmid” means at least a part containing a region essential for autonomous replication of the plasmid. The plasmid of the present invention can replicate in a host microorganism even if a region other than the region essential for the autonomous replication of the plasmid (replication control region), that is, the region other than the region containing the replication origin and genes necessary for the replication, is deleted.

The term “rep” or “repB” refers to a replication protein which controls the ability of a thermostable plasmid to replicate. As used herein the rep protein will also be referred to as a “replication protein” or a “replicase”. The term “rep” will be used to delineate the gene encoding the rep protein.

The term “origin or replication” is abbreviated “ORI” and refers to a specific site or sequence within a DNA molecule at which DNA replication is initiated. A plasmid of the invention comprises one or more ORIs. The one or more ORIs may be from any source but are preferably from bacteria or yeast. Multiple ORIs within a single plasmid may be from different sources (e.g., heterologous ORIs).

### Nucleic Acid and Amino Acid Sequences of the Invention

Applicants have identified a nucleic acid encoding a unique replication protein, repB, within the pB6A plasmid. This replication protein-encoding nucleic acid can be used in a variety of cloning and expression vectors and particularly in shuttle vectors for the expression of homologous and heterologous genes in various thermophilic hosts (e.g., Thermoanaerobacterium and Clostridium species). Comparisons of the nucleotide and amino acid sequences of the present replication protein show that the sequence is unique, having only 56.5% identity at the nucleotide level to orfB of C. butyricum plasmid pCB101 (Accession No. CAA44562, Brehm, J. K., Pennock, A., Young, M., Oultram, J. D. and Minton, N. P., “Physical characterisation of the replication origin of the cryptic plasmid pCB101 isolated from Clostridium butyricum,” Plasmid (In press)), and only 61% identity at the amino acid level to repB from the indigenous plasmid of Clostridium species MCF-1 (GenBank Accession No. U59416, Chen, T. and Leschine, S. B., Submitted (27-MAY-1996) Microbiology, Univ. of Massachusetts).

The nucleic acid sequence encoding the repB of the present invention is represented by SEQ ID NO:21:

(SEQ ID NO: 21)
atgttacaaaatgatgtttttattgattttactaataaaataaattcaataagggattgtaataaatatt
ggtatttggatgtttataaaaagcagaaaataaaggattttaaaaagactaatttgtgtaaagataa
gttctgtaataattgtaagaaagttaaacaggcttcaagaatgcaaaaatatattcctgaattacag
aaatacaaagatggcttatatcattttatatttactgttgaaaatgtgccaggtagtgaattaagaga
tactattgataggttgtttaagtctttaagtcatttacaaggtatttaagtggtaatcttaaaataaaa
ggtgttaattttgataaatggggttataaaggctgtgtaaggtctttagaggtaacttatagtatgat
tgataatcatattatgtatcatccacacttgcatgttgcgatgatattagatcctattacgatggtttt
aatgttgaaaggatgcatataattaataagtttagttatagctatggtgttttaaaaaggttgtttact
gatgatgaattattaattcaaaaaatttggtatttattgtttaataatattgaggttaacatggccaata
taaataatttagaggatggttattcttgtttagttaataagtttagtgattatgattatgcggagctgttt
aagtatatttgtaaaaatactgatgaacaaggtttacttatgacttatgatatttttaaagatttatattt
tgcattacataatgttcatcagatacaaggctatggttgtttatataatataagagatgatactcaatt
agatttaaaggttgatgacatttataatgatttgattgatttattacaagttacagaaaatcctataca
gtctatggaaactgtacaggatttattaaaggatactgaatatacaataataagccgtaagcgtat
atttaagtatctaacacaattatatcataaggat

The amino acid sequence encoding the repB protein of the present invention is represented by SEQ ID NO:22:

(SEQ ID NO: 22)

MLQNDVFIDFTNKINSIRDCNKYWYLDVYKKQKIKDFKKT
NLCKDKFCNNCKKVKQASRMQKYIPELQKYKDGLYHFIFT
VENVPGSELRDTIDRLFKSFKSFTRYLSGNLKIKGVNFDKW
GYKGCVRSLEVTYSMIDNHIMYHPHLHVAMILDPFYDGFN
VERMHIINKFSYSYGVLKRLFTDDELLIQKIWYLLFNNIEVN
MANINNLEDGYSCLVNKFSDYDYAELFKYICKNTDEQGLM
TYDIFKDLYFALHNVHQIQGYGCLYNIRDDTQLDLKVDDIY
NDLIDLLQVTENPIQSMETVQDLLKDTEYTIISRKRIFKYLTQ
LYHKD

Thus a sequence is within the scope of the invention comprises a nucleotide sequence encoding a polypeptide that has at least 70%, 75%, 80%, 85%, 90%, 95%, 96%, 97%, 98%, 99%, or 100% identity when compared to a polypeptide having the sequence as set forth in SEQ ID NO:22, or a second nucleotide sequence comprising the complement of the first nucleotide sequence. Accordingly, in some embodiments, the rep amino acid sequences are at least about 70% to about 75% identical or at least about 80% to about 85% identical to SEQ ID NO:22. In particular embodiments, the rep amino acid sequences are at least about 90% to about 95%, 96%, 97%, 98% , 99%, or 100% identical to amino acid SEQ ID NO:22. In some embodiments, the nucleotide sequence encodes a polypeptide having a replication function. In a more specific embodiment, the replication function facilitates autonomous replication of pB6A and derivative plasmids and/or vectors thereof.

Similarly, in some embodiments, nucleic acid sequences corresponding to the instant rep genes are those encoding active proteins and which are at least about 70% to about 75% identical to SEQ ID NO:21. In particular embodiments, the rep nucleic acid sequences are at least about 80% to about 85% identical to SEQ ID NO:21. In more particular embodiments, the rep nucleic acid sequences are at least about 90% to about 95%, 96%, 97%, 98%, 99%, or 100% identical SEQ ID NO:21.

In a specific embodiment, the invention is directed to an isolated nucleic acid comprising a sequence that is at least about 90% to about 95%, 96%, 97%, 98%; 99%, or 100% identical SEQ ID NO:21, provided that said sequence is not and/or does not consist only of the plasmid pB6A of SEQ ID NO:9 or the plasmid isolated from T. Saccharolyticum type strain B6A deposited as ATCC No. 49915 (DSM7060). In another specific embodiment, the invention is directed to an isolated nucleic acid comprising a sequence that encodes a polypeptide that is at least about 90% to about 95%, 96%, 97%, 98%, 99%, or 100% identical SEQ ID NO:21, provided that said sequence is not and/or does not consist only of the plasmid pB6A of SEQ ID NO:9 or the plasmid isolated from T. Saccharolyticum type strain B6A deposited as ATCC No. 49915 (DSM7060). In some embodiments the invention is directed to a plasmid comprising the isolated nucleic acid sequence. In some embodiments, the nucleotide sequence encodes a polypeptide having a replication function. In a more specific embodiment, the replication function facilitates autonomous replication of pB6A and derivative plasmids and/or vectors thereof.

There are five identified conserved domains of rolling circle Rep proteins, called Domains I-V, as well as two additional domains known as the “N” an “C” domains that are conserved for certain thermophilic Rep proteins. See Delver et al., Mol. Gen Genet (1996) 253:166-172. Delver et al. provide an amino acid sequence alignment for several Rep proteins from plasmids belonging to the pC194 family, including pCB101, which has 56.5% nucleotide sequence identity to the pB6A repB of SEQ ID NO:21, and identify the different domains within these Rep proteins. Based on the alignment of the RepB protein of SEQ ID NO:22 and pCB101, the following are the predicted domains of the RepB protein of SEQ ID NO:22:

| Conserved | Amino acid Positions of Putative RepB |
|---|---|
| Domain | Domains Within SEQ ID NO: 22 |
| I | 17-58 |
| II | 74-90 |
| III | 118-184 |
| IV | 222-242 |
| V | 248-272 |
| C | 273-313 |

Delver et al. also noted that certain thermophilic plasmids have a conserved asparagine residue in domain IV, or a histidine residue in domain II, both of which can be found in the RepB protein of SEQ ID NO:22. Another feature that is conserved in domain III among RepB proteins, including those from pCB1, pCB101, pST1 (see Delver et al., FIG. 3), and some Clostridium sp. Rep B homologs (e.g., Genbank Accession Nos. AAB02938 and AAK79836), is a “YHPHxH” motif (standard one-letter amino acid designation) in domain III of the protein. The “two His” motif (i.e., two histidines separated by a bulky hydrophobic moiety) has been recognized as conserved among numerous rolling circle initiator proteins. See, e.g., Ilyina and Koonin, Nucl. Acid. Res. (1992) 20:3279-3285.

Hence, also encompassed by the present invention are amino acid sequence fragments of the rep protein encoded by SEQ ID NO:22, wherein said fragments retain rep protein activity (e.g., functional fragments). Such fragments include, but are not limited to, conserved domains such as I-V, N, and C, as well as fragments that comprise conserved features of rolling circle Rep proteins and which confer activity to Rep proteins, such as a conserved asparagine residue in domain IV, a histidine residue in domain II, or the YHPHxH motif of domain III. Also encompassed by the present invention are nucleic acid sequences encoding the rep protein functional fragments. Also encompassed by the present invention are nucleotide and/or amino acid sequences having at least about 70%, 75%, 80%, 85%, 90%, 95%, 96%, 97%, 98%, 99%, or 100% identity the nucleotide and/or amino acid sequences encoding the rep protein functional fragments. Methods of determining the minimal replicon of a plasmid are set forth in, for example, Devine et al., J. Bateriol. (1989) 171:1166-1172. In some embodiments, the Rep proteins and functional fragments thereof can be used with any of the functional features, plasmids, vectors, heterologous sequences, etc. described herein or any combination thereof.

The present invention also comprises plasmids derived from pB6A (pMU120). The pB6A (pMU120) plasmid was isolated as described in the Examples herein from the publicly available B6A-RI type strain of Thermoanaerobacterium saccharolyticum, deposited as ATCC 49915 (ATCC, 10801 University Blvd., Manassas, Va. 20110) and DSM7060 (DSMZ, Braunschweig, Germany). The B6A type strain was deposited at ATCC in 1993, according to Lee et al., Int. J. Syst. Bacteriol. (1993) 43:41-51.

The complete nucleic acid sequence of the pB6A (pMU120) plasmid is represented by SEQ ID NO:9:

(SEQ ID NO: 9)
GGTGTTAATTTTGATAAATGGGGTTATAAAGGCTGTGTAAGGTCTTTAGAGGTAACTTATAGTATGATTGATAATCA
TATTATGTATCATCCACACTTGCATGTTGCGATGATATTAGATCCTTTTTACGATGGTTTTAATGTTGAAAGGATGC
ATATAATTAATAAGTTTAGTTATAGCTATGGTGTTTTAAAAAGGTTGTTTACTGATGATGAATTATTAATTCAAAAA
ATTTGGTATTTATTGTTTAATAATATTGAGGTTAACATGGCCAATATAAATAATTTAGAGGATGGTTATTCTTCTTT
AGTTAATAAGTTTAGTGATTATGATTATGCGGAGCTGTTTAAGTATATTTGTAAAAATACTGATGAACAAGGTTTAC
TTATGACTTATGATATTTTTAAAGATTTATATTTTGCATTACATAATGTTCATCAGATACAAGGCTATGGTTGTTTAT
ATAATATAAGAGATGATACTCAATTAGATTTAAAGGTTGATGACATTTATAATGATTTGATTGATTTATTACAAGTT
ACAGAAAATCCTATACAGTCTATGGAAACTGTACAGGATTTATTAAAGGATACTGAATATACAATAATAAGCCGTA
AGCGTATATTTAAGTATCTAACACAATTATATCATAAGGATTGATATTTATACCGTCTGTCGGACTCATGCGGAGG
GGGACTTGAGGGGGTCTCCCCTCGCATTGTACGACAGACGGTATTATTATTATACAAATTTTTTTTATGTAATTTTTT
TTGTGTAATTTTTTTATACAAATAATATTTCAATTGACAAAGTTTTCTATTTGTGTTAACATTGTTTATATAATAGTG
AACAGTGTTAAGATTAAATGTGAGGTGTTTGTATGGATATTAATGATTATAAAGAGAAGGGACTTTATTTATTAAG
TAGTATGGATGATTTTATTAAAATTAATGATTTGTTTATGGGTAAAGTTGTTTCTCCTGGCTATGTTGCTTCGGTTTT
TGGTGTTTCCAGGTCTACTGTTACACAATGGATTCAAAGACGTAAAATTAGAGCTTTTAAGTATAAAGGTAAGGAA
GGTGACTATATGGTTATACCTATTGCTGATATTATTGATTACAAAAGATTGAGTAATAATGATTTTATTTATGATAA
GTTAGTGAGGTGATTTATTTTATGTTTGACGATAGCTATGTTGTTAATGAGTGTTCGTCTAATGTTAGTGAAAATGA
TAGAGATTTTTGTAGTTTGGTTGGTCGTTTTATGATTATTAATGGTATAGATAAGTTGGTTATTAAGATTAATAGAA
AATTTAATAGGAAATCTTTAAGTTTAGATTTTAGTGTTGATTTATTCCCTTCTATCAAAGTTTCTGAATAGTTTTTT
TTGATGAGTTTAACAAAACGTGTGGTTTTTATTTTTCTTTTAATTCTTTTACAATTTTTAAGGCTTTTAGAGATGTTC
ATAATCATAATAAAATATCATTTTATTTTGCATAATTTCGGGTCTGGGCCGCAGACCAGGCCCAGTGCTAACAATAT
TAATTTTTAATGTTAGGAATTGTTTAATTCTTAATTGTGTTTTTAAAGGTAGAATAATTACCCATTCGCCCTTTAGCC
AACAAAAATTAAGGAGGTATAAACATGGATAAAATGGATTTGATTCTTCAAGATGAAAGACTGGGTGAGATATTT
AAAGATATAGATTTAACAGATAATGAAAAGAGATATCTTAAATGGTTATGGAAATGGGATTATGAAACACGTGAT
ACTTTTGTATCAATTTTTTTGAAGCTAAAAAATGGTGGAAAATGATTTTTTTCTTATCTTGATATATTAGAAAAAAG
CGTACTCACGAAGTAAGAATTTGTAAAAAAAGAAGGGGGGATTTTTTTGGATGAGAGTTTGTACAAGCAGATTTTA
AGTAATATTATTATTACTCGTGATTATTGTAAAAATGTTTTAGATAATATAAAGTTCAATGAAAAAATAATTGATTA
TTATGTTATGTTACAAAATGATGTTTTTATTGATTTTACTAATAAAATAAATTCAATAAGGGATTGTAATAAATATT
GGTATTTGGATGTTTATAAAAAGCAGAAAATAAAGGATTTTAAAAAGACTAATTTGTGTAAAGATAAGTTCTGTAA
TAATTGTAAGAAAGTTAAACAGGCTTCAAGAATGCAAAAATATATTCCTGAATTACAGAAATACAAAGATGGCTT
ATATCATTTTATATTTACTGTTGAAAATGTGCCAGGTAGTGAATTAAGAGATACTATTGATAGGTTGTTTAAGTCTT
TTAAGTCATTTACAAGGTATTTAAGTGGTAATCTTAAAATAAAA

The present invention also encompass a nucleic acid comprising a sequence that is at least about 70%, 75%, or 80% identical, preferably at least about 90% to about 95% identical, and more preferably at least about 96%, 97%, 98%, 99% or 100% identical to SEQ ID NO:9. In some embodiments, the present invention is directed to isolated nucleotide sequences that are not and/or do not consist only of the plasmid pB6A of SEQ ID NO:9 or the plasmid isolated from T. Saccharolyticum type strain B6A deposited as ATCC No. 49915 (DSM7060). In particular embodiments, plasmids derived from pB6A may comprise any of functional units or heterologous sequence described herein or any combination thereof.

The nucleic acid sequences and fragments thereof of the present invention may be used to isolate genes encoding homologous proteins from the same or other microbial species. Isolation of homologous genes using sequence-dependent protocols is well known in the art. Examples of sequence-dependent protocols include, but are not limited to, methods of nucleic acid hybridization, and methods of DNA and RNA amplification as exemplified by various uses of nucleic acid amplification technologies (e.g., polymerase chain reaction, Mullis et al., U.S. Pat. No. 4,683,202; ligase chain reaction (LCR) (Tabor, S. et al., Proc. Acad. Sci. USA 82, 1074, (1985)); or strand displacement amplification (S D A, Walker, et al., Proc. Natl. Acad. Sci. U.S.A., 89, 392, (1992)).

For example, genes encoding similar proteins or polypeptides to those of the instant invention could be isolated directly by using all or a portion of the instant nucleic acid fragments as DNA hybridization probes to screen libraries from any desired bacteria using methodology well known to those skilled in the art. Specific oligonucleotide probes based upon the instant nucleic acid sequences can be designed and synthesized by methods known in the art (see, e.g., Maniatis, supra 1989). Moreover, the entire sequences can be used directly to synthesize DNA probes by methods known to the skilled artisan such as random primers DNA labeling, nick translation, or end-labeling techniques, or RNA probes using available in vitro transcription systems. In addition, specific primers can be designed and used to amplify a part of or full-length of the instant sequences. The resulting amplification products can be labeled directly during amplification reactions or labeled after amplification reactions, and used as probes to isolate full length DNA fragments under conditions of appropriate stringency.

Typically, in PCR-type amplification techniques, the primers have different sequences and are not complementary to each other. Depending on the desired test conditions, the sequences of the primers should be designed to provide for both efficient and faithful replication of the target nucleic acid. Methods of PCR primer design are common and well known in the art. Generally two short segments of the instant sequences may be used in polymerase chain reaction (PCR) protocols to amplify longer nucleic acid fragments encoding homologous genes from DNA or RNA. The polymerase chain reaction may also be performed on a library of cloned nucleic acid fragments wherein the sequence of one primer is derived from the instant nucleic acid fragments, and the sequence of the other primer takes advantage of the presence of the polyadenylic acid tracts to the 3′ end of the mRNA precursor encoding microbial genes. Alternatively, the second primer sequence may be based upon sequences derived from the cloning vector. For example, the skilled artisan can follow the RACE protocol (Frohman et al., PNAS USA 85:8998 (1988)) to generate cDNAs by using PCR to amplify copies of the region between a single point in the transcript and the 3′ or 5′ end. Primers oriented in the 3′ and 5′ directions can be designed from the instant sequences. Using commercially available 3′ RACE or 5′ RACE systems (BRL), specific 3′ or 5′ cDNA fragments can be isolated (Ohara et al., PNAS USA 86:5673 (1989); Loh et al., Science 243:217 (1989)).

Alternatively the instant sequences may be employed as hybridization reagents for the identification of homologs. The basic components of a nucleic acid hybridization test include a probe, a sample suspected of containing the gene or gene fragment of interest, and a specific hybridization method. Probes of the present invention are typically single stranded nucleic acid sequences which are complementary to the nucleic acid sequences to be detected. Probes are “hybridizable” to the nucleic acid sequence to be detected. The probe length can vary from 5 bases to tens of thousands of bases, and will depend upon the specific test to be done. Typically a probe length of about 15 bases to about 30 bases is suitable. Only part of the probe molecule need be complementary to the nucleic acid sequence to be detected. In addition, the complementarity between the probe and the target sequence need not be perfect. Hybridization does occur between imperfectly complementary molecules with the result that a certain fraction of the bases in the hybridized region are not paired with the proper complementary base.

Hybridization methods are well defined and have been described above. Nucleic acid hybridization is adaptable to a variety of assay formats. One of the most suitable is the sandwich assay format. The sandwich assay is particularly adaptable to hybridization under non-denaturing conditions. A primary component of a sandwich-type assay is a solid support. The solid support has adsorbed to it or covalently coupled to it immobilized nucleic acid probe that is unlabeled and complementary to one portion of the sequence.

### Plasmids and Vectors of the Invention

Plasmids useful for gene expression in microorganisms may be either self-replicating (autonomously replicating) plasmids or chromosomally integrated. The self-replicating plasmids have the advantage of having multiple copies of genes of interest, and therefore the expression level can be very high. Chromosome integration plasmids are integrated into the genome by recombination. They have the advantage of being transmitted through successive generations as part of the host chromosome, but they may suffer from a lower level of expression. In a preferred embodiment, plasmids or vectors according to the present invention are stable and self-replicating and are used according to the methods of the invention.

Vectors or plasmids useful for the transformation of suitable host cells are well known in the art. Typically the vector or plasmid contains sequences directing transcription and translation of the relevant gene, a selectable marker, and sequences allowing autonomous replication or chromosomal integration. In a specific embodiment, the plasmid or vector comprises a nucleic acid according to the present invention. Suitable vectors comprise a region 5′ of the gene which harbors transcriptional initiation controls and a region 3′ of the DNA fragment which controls transcriptional termination. It some embodiments, both control regions are derived from genes homologous to the transformed host cell, however, such control regions need not be derived from the genes native to the specific species chosen as a production host.

Vectors of the present invention will additionally contain a unique replication protein (rep), as described above, that facilitates the replication of the vector in the thermophilic host. Additionally the present vectors will comprise a stability coding sequence that is useful for maintaining the stability of the vector in the host and has a significant degree of homology to putative cell division proteins. The vectors of the present invention will contain convenient restriction sites for the facile insertion of genes of interest to be expressed in a thermophilic host.

In a preferred embodiment, the vectors of the present invention comprise one or more restriction sites. In one embodiment, the vectors comprise a multiple cloning site (MCS) comprising one or more unique restriction sites. Non-limiting examples of the restriction sites for use in the present invention include sites for recognition by HindIII, MluI, SpeI, BglII, StuI, BspDI/ClaI, PvuII, NdeI, NcoI, SmaI/XmaI, SacII, PvuI, EagI/XmaIII, PaeR7I/XhoI, PstI, EcoRI, SqacI, EcoRV, SphI, NaeI, NheI, BamHI, NarI, ApaI, Acc65I/KpnI, SalI, ApaLI, HpaI, BspEI, NruI, XbaI, BclI, BalI, SwaI, Sse8387I, SrfI, NotI, AscI, PacI, and PmeI, or any combination thereof. In a particular embodiment, the EcoRI, SacI, KpnI, SmaI, XmaI, BamHI, XbaI, HincII, PstI, SphI, HindIII, AvaI, or any combination thereof.

The present invention relates to a specific plasmid, pB6A (pMU120), isolated from a Thermoanaerobacterium saccharolyticum host, and plasmids and shuttle vectors derived and constructed therefrom. The pB6A vector contains a unique replication sequence for Thermoanaerobacterium, while the shuttle vectors additionally contain an origin of replication (ORI) for replication in E. coli and antibiotic resistance markers for selection in thermophilic hosts and E. coli.

Bacterial plasmids typically range in size from about 1 kb to about 200 kb and are generally autonomously replicating genetic units in the bacterial host. When a bacterial host has been identified that may contain a plasmid containing desirable genes, cultures of host cells are grown up, lysed and the plasmid purified from the cellular material. If the plasmid is of the high copy number variety, it is possible to purify it without additional amplification. If additional plasmid DNA is needed, a bacterial cell may be grown in the presence of a protein synthesis inhibitor such as chloramphenicol which inhibits host cell protein synthesis and allow additional copies of the plasmid to be made. Cell lysis may be accomplished either enzymatically (e.g., lysozyme) in the presence of a mild detergent, by boiling or treatment with strong base. The method chosen will depend on a number of factors including the characteristics of the host bacteria and the size of the plasmid to be isolated.

After lysis, the plasmid DNA may be purified by gradient centrifugation (CsCl-ethidium bromide for example) or by phenol:chloroform solvent extraction. Additionally, size or ion exchange chromatography may be used as well as differential separation with polyethylene glycol. Readily available commercial plasmid prep kits may also be used.

Once the plasmid DNA has been purified, the plasmid may be analyzed by restriction enzyme analysis and sequenced to determine the sequence of the genes contained on the plasmid and the position of each restriction site to create a plasmid restriction map. Methods of constructing or isolating vectors are common and well known in the art (see, e.g., Maniatis, supra, Chapter 1; Rohde, C., World J. Microbiol. Biotechnol. (1995), 11(3), 367-9); Trevors, J. T., J. Microbiol. Methods (1985), 3(5-6), 259-71).

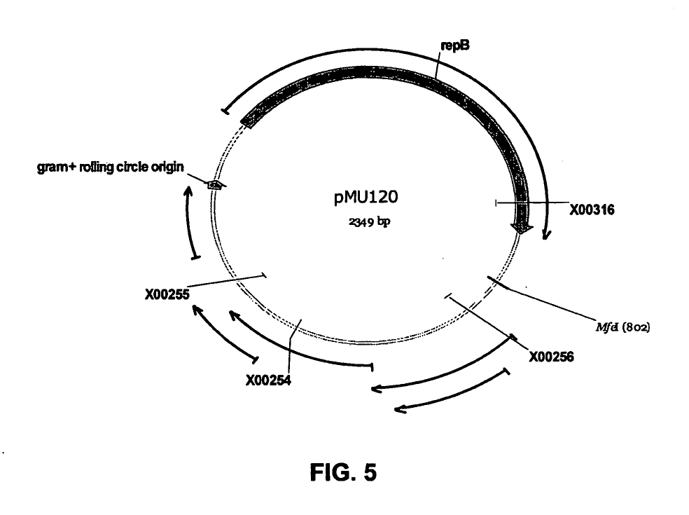

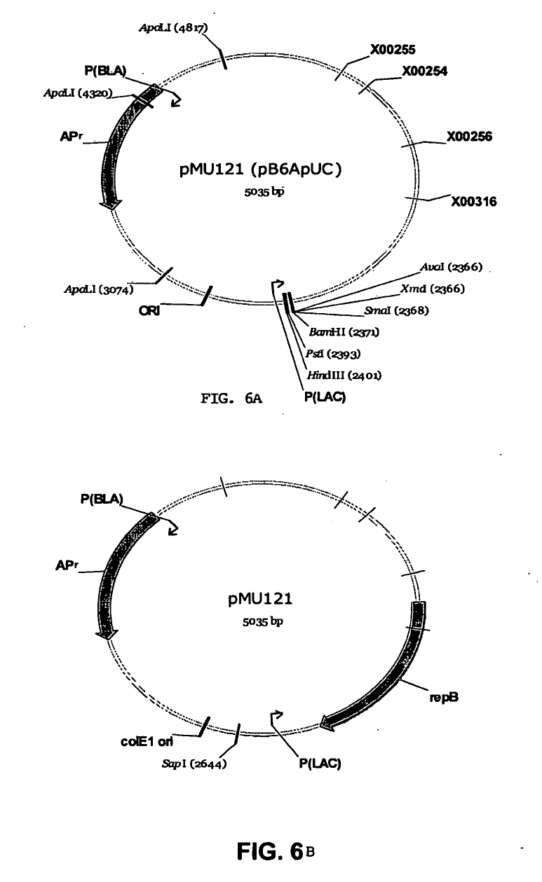

Using standard methods, the 2.3 kb pB6A (pMU120) was isolated from Thermoanaerobacterium saccharolyticm strain B6A (ATCC Deposit 49915/DSM7060), purified, and mapped to identify six open reading frames (see FIG. 5), as described in the Examples herein.

Once mapped, isolated plasmids may be modified in a number of ways. Using the existing restriction sites, specific genes desired for expression in the host cell may be inserted within the plasmid. Additionally, using techniques well known in the art, new or different restriction sites may be engineered into the plasmid to facilitate gene insertion. Many native bacterial plasmids contain genes encoding resistance or sensitivity to various antibiotics. However, it may be useful to insert additional selectable markers to replace the existing ones with others. Selectable markers useful in the present invention include, but are not limited to genes conferring antibiotic resistance or sensitivity, genes encoding a selectable label such as a color (e.g., lac) or light (e.g., Luc; Lux) or genes encoding proteins that confer a particular phenotypic metabolic or morphological trait. Generally, markers that are selectable in both gram negative and gram positive hosts are preferred. Particularly suitable in the present invention are markers that encode antibiotic resistance or sensitivity, including but not limited to ampicillin resistance gene, tetracycline resistance gene, erythromycin resistance gene, chloramphenicol resistance gene, kanamycin resistance gene, and thiostrepton resistance gene.

In one aspect, plasmids of the present invention contain a gene of interest to be expressed in the host. The genes to be expressed may be either native or endogenous to the host or foreign or heterologous genes. Particularly suitable are genes encoding enzymes or proteins (or functional fragments thereof) involved in various synthesis or degradation pathways. In one embodiment, the gene of interest encodes a protein or functional fragment thereof that facilitates the anaerobic oxidation of an organic compound.

Genes of interest for expression in a thermophilic host (e.g., Thermoanaerobacterium or Clostridium) using Applicants' vectors and methods include, but are not limited to: endoglucanase, exoglucanase, endoxylanase, exoxylanase, endogalactanase, endoarabinase, cellobiohydrolase, exo-β-1,3-glucanase, endo-β-1,4-glucanase, endo-β-D-mannanase, endo-β-1,4-mannanase, β-mannanase, β-mannosidase, endo-β-xylanase, α-galactosidase, polygalacturonase, α-glucuronidase, cellodextrinase, xyloglucanase, xylose isomerase, xylose reductase, xylitol dehydrogenase, xylulokinase, transaldolase, transketolase, β-glucosidase, endo-1,4-β-xylanase (EC-Number 3.2.1.8), xylan endo-β-1,3-xylosidase (EC-Number 3.2.1.32), α-xylosidase, β-xylosidase, oligoxyloglucan hydrolase, oligoxyloglucan reducing-end-specific cellobiohydrolase (EC-Number 3.2.1.150), endoxyloglucan transferase, xyloglucan endotransglycosylase, xyloglucan hydrolase, xyloglucan endohydrolase, xyloglucan-specific exo-β-1,4-glucanase (EC-Number 3.2.1.155), xyloglucan-specific endo-β-1,4-glucanase (EC-Number 3.2.1.151), glucuronoarabinoxylan endo-β-1,4-xylanase (EC-Number 3.2.1.136), α-L-arabinofuranosidase, acetylesterase, acetylxylanesterase, α-amylase, β-amylase, glucoamylase, pullulanase, β-glucanase, hemicellulase, arabinosidase, mannanase, pectin hydrolase, pectate lyase, and combinations thereof.

The plasmids or vectors according to the invention may further comprise at least one promoter suitable for driving expression of a gene in a thermophilic host (e.g., Thermoanaerobacterium or Clostridium). Typically these promoters, including the initiation control regions, will be derived from the thermophilic host. Termination control regions may also be included and may be derived from various genes native to the preferred hosts.

Optionally it may be desired to produce the instant gene product as a secretion product of the transformed host. Secretion of desired proteins into the growth media has the advantages of simplified and less costly purification procedures. It is well known in the art that secretion signal sequences are often useful in facilitating the active transport of expressible proteins across cell membranes. The creation of a transformed host capable of secretion may be accomplished by the incorporation of a DNA sequence that codes for a secretion signal which is functional in the host production host. Methods for choosing appropriate signal sequences are well known in the art (see for example EP 546049; WO 9324631). The secretion signal DNA or facilitator may be located between the expression-controlling DNA and the instant gene or gene fragment, and in the same reading frame with the latter.

Aspects of the present invention relate to the transformation of thermophilic microorganisms with plasmids and vectors of the present invention. Their potential in process applications in biotechnology stems from their ability to grow at relatively high temperatures with attendant high metabolic rates, production of physically and chemically stable enzymes, and elevated yields of end products. Major groups of thermophilic bacteria include eubacteria and archaebacteria. Thermophilic eubacteria include: phototropic bacteria, such as cyanobacteria, purple bacteria, and green bacteria; Gram-positive bacteria, such as Bacillus, Clostridium, Lactic acid bacteria, and Actinomyces; and other eubacteria, such as Thiobacillus, Spirochete, Desulfotomaculum, Gram-negative aerobes, Gram-negative anaerobes, and Thermotoga. Within archaebacteria are considered Methanogens, extreme thermophiles (an art-recognized term), and Thermoplasma. In certain embodiments, the present invention relates to Gram-negative organotrophic thermophiles of the genera Thermus, Gram-positive eubacteria, such as genera Clostridium, and also which comprise both rods and cocci, genera in group of eubacteria, such as Thermosipho and Thermotoga, genera of Archaebacteria, such as Thermococcus, Thermoproteus (rod-shaped), Thermofilum (rod-shaped), Pyrodictium, Acidianus, Sulfolobus, Pyrobaculum, Pyrococcus, Thermodiscus, Staphylothermus, Desulfurococcus, Archaeoglobus, and Methanopyrus. Some examples of thermophilic microorganisms (including bacteria, prokaryotic microorganism, and fungi), which may be suitable for the present invention include, but are not limited to: Clostridium thermosulfurogenes, Clostridium cellulolyticum, Clostridium thermocellum, Clostridium thermohydrosulfuricum, Clostridium thermoaceticum, Clostridium thermosaccharolyticum, Clostridium tartarivorum, Clostridium thermocellulaseum, Thermoanaerobacterium thermosaccarolyticum, Thermoanaerobacterium saccharolyticum, Thermobacteroides acetoethylicus, Thermoanaerobium brockii, Methanobacterium thermoautotrophicum, Pyrodictium occultum, Thermoproteus neutrophilus, Thermofilum librum, Thermothrix thioparus, Desulfovibrio thermophilus, Thermoplasma acidophilum, Hydrogenomonas thermophilus, Thermomicrobium roseum, Thermus flavas, Thermus ruber, Pyrococcus furiosus, Thermus aquaticus, Thermus thermophilus, Chloroflexus aurantiacus, Thermococcus litoralis, Pyrodictium abyssi, Bacillus stearothermophilus, Cyanidium caldarium, Mastigocladus laminosus, Chlamydothrix calidissima, Chlamydothrix penicillata, Thiothrix carnea, Phormidium tenuissimum, Phormidium geysericola, Phormidium subterraneum, Phormidium bijahensi, Oscillatoria filiformis, Synechococcus lividus, Chloroflexus aurantiacus, Pyrodictium brockii, Thiobacillus thiooxidans, Sulfolobus acidocaldarias, Thiobacillus thermophilica, Bacillus stearothermophilus, Cercosulcifer hamathensis, Vahlkampfia reichi, Cyclidium citrullus, Dactylaria gallopava, Synechococcus lividus, Synechococcus elongatus, Synechococcus minervae, Synechocystis aquatilus, Aphanocapsa thermalis, Oscillatoria terebriformis, Oscillatoria amphibia, Oscillatoria germinata, Oscillatoria okenii, Phormidium laminosum, Phormidium parparasiens, Symploca thermalis, Bacillus acidocaldarias, Bacillus coagulans, Bacillus thermocatenalatus, Bacillus licheniformis, Bacillus pamilas, Bacillus macerans, Bacillus circulans, Bacillus laterosporus, Bacillus brevis, Bacillus subtilis, Bacillus sphaericus, Desulfotomaculum nigrificans, Streptococcus thermophilus, Lactobacillus thermophilus, Lactobacillus bulgaricus, Bifidobacterium thermophilum, Streptomyces fragmentosporus, Streptomyces thermonitrificans, Streptomyces thermovulgaris, Pseudonocardia thermophila, Thermoactinomyces vulgaris, Thermoactinomyces sacchari, Thermoactinomyces candidas, Thermomonospora curvata, Thermomonospora viridis, Thermomonospora citrina, Microbispora thermodiastatica, Microbispora aerata, Microbispora bispora, Actinobifida dichotomica, Actinobifida chromogena, Micropolyspora caesia, Micropolyspora faeni, Micropolyspora cectivugida, Micropolyspora cabrobrunea, Micropolyspora thermovirida, Micropolyspora viridinigra, Methanobacterium thermoautothropicum, variants thereof, and/or progeny thereof.

In certain embodiments, the present invention relates to thermophilic bacteria of the genera Thermoanaerobacterium or Thermoanaerobacter, including, but not limited to, species selected from the group consisting of: Thermoanaerobacterium thermosulfurigenes, Thermoanaerobacterium aotearoense, Thermoanaerobacterium polysaccharolyticum, Thermoanaerobacterium zeae, Thermoanaerobacterium xylanolyticum, Thermoanaerobacterium saccharolyticum, Thermoanaerobium brockii, Thermoanaerobacterium thermosaccharolyticum, Thermoanaerobacter thermohydrosulfuricus, Thermoanaerobacter ethanolicus, Thermoanaerobacter brockii, variants thereof, and progeny thereof.

In certain embodiments, the present invention relates to microorganisms of the genera Geobacillus, Saccharococcus, Paenibacillus, Bacillus, and Anoxybacillus, including, but not limited to, species selected from the group consisting of Geobacillus thermoglucosidasius, Geobacillus stearothermophilus, Saccharococcus caldoxylosilyticus, Saccharoccus thermophiles, Paenibacillus campinasensis, Bacillus flavothermus, Anoxybacillus kamchatkensis, Anoxybacillus gonensis, variants thereof, and progeny thereof.

The present invention also relates to a plasmid or vector that is able to replicate or “shuttle” between at least two different organisms. Shuttle vectors are useful for carrying genetic material from one organism to another. The shuttle vector is distinguished from other vectors by its ability to replicate in more than one host. This is facilitated by the presence of an origin of replication corresponding to each host in which it must replicate. The present vectors are designed to replicate in thermophilic hosts for the purpose of gene expression. As such, each will contain an ORI capable of initiating replication in the host (e.g., Thermoanaerobacterium or Clostridium, or any other thermophilic bacteria or yeast host, including but not limited to those listed herein). Many of the genetic manipulations for this vector may be easily accomplished in E. coli. It is therefore particularly useful to have a shuttle vector comprising an origin of replication that will function in E. coli and other gram positive bacteria. A number of ORI sequences for gram positive bacteria have been determined and the sequence for the ORI in E. coli determined (see for example Hirota et al., Prog. Nucleic Acid Res. Mol. Biol. (1981), 26, 33-48); Zyskind, J. W.; Smith, D. W., Proc. Natl. Acad. Sci. U.S.A., 77, 2460-2464 (1980), GenBank ACC. NO. (GBN): J01808). In some embodiments, the ORI sequences are isolated from gram positive bacteria, and particularly those members of the Actinomycetales bacterial family. Members of the Actinomycetales bacterial family include for example, the genera Actinomyces, Actinoplanes, Arcanobacterium, Corynebacterium, Dietzia, Gordonia, Mycobacterium, Nocardia, Rhodococcus, Tsukamurella, Brevibacterium, Arthrobacter, Propionibacterium, Streptomyces, Micrococcus, and Micromonospora. In other embodiments, the ORI sequences are isolated or derived from other bacterial or yeast cell hosts including, but not limited to the genera and species of bacteria and yeast listed herein above.

In one aspect, the present invention is directed to a method of producing a shuttle vector, the method comprising: providing a first replicon that is autonomously replicable in a first host, the replicon comprising a nucleotide sequence encoding a polypeptide having Rep protein activity, wherein the nucleotide sequence is at least about 70%, 75%, 80%, 85%, 90%, 95%, 96%, 97%, 98%, 99% or 100% identical to the nucleotide sequence of SEQ ID NO:21 or a functional fragment thereof and/or wherein the polypeptide encoded by the nucleotide is at least about 70%, 75%, 80%, 85%, 90%, 95%, 96%, 97%, 98%, 99% or 100% identical to the amino acid sequence of SEQ ID NO:22 (also included for use in the shuttle vector and methods are those functional fragments of the Rep protein as described in detail herein above); digesting the first replicon with one or more restriction enzymes to obtain a fragment of the replicon comprising at least the nucleotide sequence encoding a polypeptide having Rep protein activity; digesting a second (or third, or fourth, etc.) replicon that is heterologous to the first replicon and autonomously replicable in a second host with one or more restriction enzymes to obtain a fragment of the second (or third, or fourth, etc.) replicon comprising at least an origin of replication; ligating the fragments to obtain a shuttle vector that is autonomously replicable in both the first host and the second (or third, or fourth, etc.) host. The method can be performed using standard molecular biology techniques as know in the art and described herein.

In a particular embodiment, the first replicon is pB6A (pMU120) as represented by SEQ ID NO:9 or the plasmid isolated from the T. Saccharolyticum type strain deposited as ATCC 49915/DSM7060, or a derivative or variant thereof. In another particular embodiment, the second (or third, fourth, etc.) replicon is capable of replicating in a bacterial host. In a preferred embodiment, the bacterial host is E. coli. In a specific embodiment, the second (or third, fourth, etc.) replicon is selected from the group consisting of ColE1, pMB1, p15A, pSC101, F, R6K, R1, RK2, pRO1600, and λ dv. In another specific embodiment, the second (or third, fourth, etc.) replicon is a plasmid selected from the group consisting of pUC19, pUC18, pBR322, pMK16, pACYC184, pLG338, pDF41, pRK353, pBEU50, pRK2501, pGE374, pTrc99A, pTrc99B, and pTrc99C. In another particular embodiment, the second (or third, fourth, etc.) replicon is capable of replicating in a yeast host cell. In one embodiment, the yeast host cell is Saccharomyces cerevisie. In a particular embodiment, the second (or third, fourth, etc.) replicon is a yeast replicon selected from the group consisting of: ARS 1 and the 2 μm replicon. In another specific embodiment, the second (or third, fourth, etc.) replicon is a yeast plasmid selected from the group consisting of YIp5, YRp7, YRp17, YEp13, YEp24, YCp19, YCp50, YLp21, pYAC3, CEN4, and 2 μm plasmid.

Shuttle vectors of the present invention can also comprise one or more heterologous nucleotide sequences encoding one or more proteins or functional protein fragments, including but not limited to proteins of interest described herein; one or multiple cloning sites (polylinkers); and one or more restriction sites in addition to those found in the multiple cloning site. In a particular embodiment, the shuttle vectors of the present invention comprise one or more selectable markers.

In specific embodiments, numerous shuttle vectors are described herein: pMU121, pMU131, pMU141, pMU143, pMU144, and pMU362, each of which is based on ligation of pMU120 with pUC19, with the addition of various selection markers, and pMU158, pMU166, and pMU675, which also include a yeast replicon.

pMU121 has a size of about 5 kb and its map is shown in FIG. 6. The complete sequence of pMU121 is given in SEQ ID NO:10:

(SEQ ID NO: 10)
AATTGACAAAGTTTTCTATTTGTGTTAACATTGTTTATATAATAGTGAACAGTGTTAAGATTAAATGTGAGGTGTTT
GTATGGATATTAATGATTATAAAGAGAAGGGACTTTATTTATTAAGTAGTATGGATGATTTTATTAAAATTAATGAT
TTGTTTATGGGTAAAGTTGTTTCTCCTGGCTATGTTGCTTCGGTTTTTGGTGTTTCCAGGTCTACTGTTACACAATGG
ATTCAAAGACGTAAAATTAGAGCTTTTAAGTATAAAGGTAAGGAAGGTGACTATATGGTTATACCTATTGCTGATA
TTATTGATTACAAAAGATTGAGTAATAATGATTTTATTTATGATAAGTTAGTGAGGTGATTTATTTTATGTTTGACG
ATAGCTATGTTGTTAATGAGTGTTCGTCTAATGTTAGTGAAAATGATAGAGATTTTTGTAGTTTGGTTGGTCGTTTT
ATGATTATTAATGGTATAGATAAGTTGGTTATTAAGATTAATAGAAAATTTAATAGGAAATCTTTAAGTTTAGATTT
TAGTGTTGATTTATTCCCTTCTATCAAAGTTTCTGAATTAGTTTTTTTTGATGAGTTTAACAAAACGTGTGGTTTTTA
TTTTTCTTTTAATTCTTTTACAATTTTTAAGGCTTTTAGAGATGTTCATAATCATAATAAAATATCATTTTATTTTGCA
TAATTTCGGGTCTGGGCCGCAGACCAGGCCCAGTGCTAACAATATTAATTTTTAATGTTAGGAATTGTTTAATTCTT
AATTGTGTTTTTAAAGGTAGAATAATTACCCATTCGCCCTTTAGCCAACAAAAATTAAGGAGGTATAAACATGGAT
AAAATGGATTTGATTCTTCAAGATGAAAGACTGGGTGAGATATTTAAAGATATAGATTTAACAGATAATGAAAAG
AGATATCTTAAATGGTTATGGAAATGGGATTATGAAACACGTGATACTTTTGTATCAATTTTTTGAAGCTAAAAAA
TGGTGGAAAATGATTTTTTTCTTATCTTGATATATTAGAAAAAAGCGTACTCACGAAGTAAGAATTTGTAAAAAAA
GAAGGGGGGATTTTTTTGGATGAGAGTTTGTACAAGCAGATTTTAAGTAATATTATTATTACTCGTGATTATTGTAA
AAATGTTTTAGATAATATAAAGTTCAATGAAAAAATAATTGATTATTATGTTATGTTACAAAATGATGTTTTTATTG
ATTTTACTAATAAAATAAATTCAATAAGGGATTGTAATAAATATTGGTATTTGGATGTTTATAAAAAGCAGAAAAT
AAAGGATTTTAAAAAGACTAATTTGTGTAAAGATAAGTTCTGTAATAATTGTAAGAAAGTTAAACAGGCTTCAAGA
ATGCAAAAATATATTCCTGAATTACAGAAATACAAAGATGGCTTATATCATTTTATATTTACTGTTGAAAATGTGCC
AGGTAGTGAATTAAGAGATACTATTGATAGGTTGTTTAAGTCTTTTAAGTCATTTACAAGGTATTTAAGTGGTAATC
TTAAAATAAAAGGTGTTAATTTTGATAAATGGGGTTATAAAGGCTGTGTAAGGTCTTTAGAGGTAACTTATAGTAT
GATTGATAATCATATTATGTATCATCCACATTGCATGTTGCGATGATATTAGATCCTTTTTACGATGGTTTTAATGT
TGAAAGGATGCATATAATTAATAAGTTTAGTTATAGCTATGGTGTTTTAAAAAGGTTGTTTACTGATGATGAATTAT
TAATTCAAAAAATTTGGTATTTATTGTTTAATAATATTGAGGTTAACATGGCCAATATAAATAATTTAGAGGATGGT
TATTCTTGTTTAGTTAATAAGTTTAGTGATTATGATTATGCGGAGCTGTTTAAGTATATTTGTAAAAATACTGATGA
ACAAGGTTTACTTATGACTTATGATATTTTTAAAGATTTATATTTTGCATTACATAATGTTCATCAGATACAAGGCT
ATGGTTGTTTATATAATATAAGAGATGATACTCAATTAGATTTAAAGGTTGATGACATTTATAATGATTTGATTGAT
TTATTACAAGTTACAGAAAATCCTATACAGTCTATGGAAACTGTACAGGATTTATTAAAGGATACTGAATATACAA
TAATAAGCCGTAAGCGTATATTTAAGTATCTAACACAATTATATCATAAGGATTGATATTTATACCGTCTGTCGGAC
TCATGCGGAGGGGGACTTGAGGGGGTCTCCCCTCGCATTGTACGACAGACGGTATTATTATTATACAAATTTTTTTT
ATGTAATTTTTTTTGTGTAATTTTTTTATACAAATAATATTTCAATTCGAGCTCGGTACCCGGGGATCCTCTAGAGTC
GACCTGCAGGCATGCAAGCTTGGCGTAATCATGGTCATAGCTGTTTCCTGTGTGAAATTGTTATCCGCTCACAATTC
CACACAACATACGAGCCGGAAGCATAAAGTGTAAAGCCTGGGGTGCCTAATGAGTGAGCTAACTCACATTAATTG
CGTTGCGCTCACTGCCCGCTTTCCAGTCGGGAAACCTGTCGTGCCAGCTGCATTAATGAATCGGCCAACGCGCGGG
GAGAGGCGGTTTGCGTATTGGGCGCTCTTCCGCTTCCTCGCTCACTGACTCGCTGCGCTCGGTCGTTCGGCTGCGGC
GAGCGGTATCAGCTCACTCAAAGGCGGTAATACGGTTATCCACAGAATCAGGGGATAACGCAGGAAAGAACATGT
GAGCAAAAGGCCAGCAAAAGGCCAGGAACCGTAAAAAGGCCGCGTTGCTGGCGTTTTTCCATAGGCTCCGCCCCC
CTGACGAGCATCACAAAAATCGACGCTCAAGTCAGAGGTGGCGAAACCCGACAGGACTATAAAGATACCAGGCGT
TTCCCCCTGGAAGCTCCCTCGTGCGCTCTCCTGTTCCGACCCTGCCGCTTACCGGATACCTGTCCGCCTTTCTCCCTT
CGGGAAGCGTGGCGCTTTCTCATAGCTCACGCTGTAGGTATCTCAGTTCGGTGTAGGTCGTTCGCTCCAAGCTGGG
CTGTGTGCACGAACCCCCCGTTCAGCCCGACCGCTGCGCCTTATCCGGTAACTATCGTCTTGAGTCCAACCCGGTAA
GACACGACTTATCGCCACTGGCAGCAGCCACTGGTAACAGGATTAGCAGAGCGAGGTATGTAGGCGGTGCTACAG
AGTTCTTGAAGTGGTGGCCTAACTACGGCTACACTAGAAGGACAGTATTTGGTATCTGCGCTCTGCTGAAGCCAGT
TACCTTCGGAAAAAGAGTTGGTAGCTCTTGATCCGGCAAACAAACCACCGCTGGTAGCGGTGGTTTTTTTGTTTGC
AAGCAGCAGATTACGCGCAGAAAAAAAGGATCTCAAGAAGATCCTTTGATCTTTTCTACGGGGTCTGACGCTCAGT
GGAACGAAAACTCACGTTAAGGGATTTTGGTCATGAGATTATCAAAAAGGATCTTCACCTAGATCCTTTTAAATTA
AAAATGAAGTTTTAAATCAATCTAAAGTATATATGAGTAAACTTGGTCTGACAGTTACCAATGCTTAATCAGTGAG
GCACCTATCTCAGCGATCTGTCTATTTCGTTCATCCATAGTTGCCTGACTCCCCGTCGTGTAGATAACTACGATACG
GGAGGGCTTACCATCTGGCCCCAGTGCTGCAATGATACCGCGAGACCCACGCTCACCGGCTCCAGATTTATCAGCA
ATAAACCAGCCAGCCGGAAGGGCCGAGCGCAGAAGTGGTCCTGCAACTTTATCCGCCTCCATCCAGTCTATTAATT
GTTGCCGGGAAGCTAGAGTAAGTAGTTCGCCAGTTAATAGTTTGCGCAACGTTGTTGCCATTGCTACAGGCATCGT
GGTGTCACGCTCGTCGTTTGGTATGGCTTCATTCAGCTCCGTTCCCAACGATCAAGGCGAGTTACATGATCCCCCA
TGTTGTGCAAAAAAGCGGTTAGCTCCTTCGGTCCTCCGATCGTTGTCAGAAGTAAGTTGGCCGCAGTGTTATCACTC
ATGGTTATGGCAGCACTGCATAATTCTCTTACTGTCATGCCATCCGTAAGATGCTTTTCTGTGACTGGTGAGTACTC
AACCAAGTCATTCTGAGAATAGTGTATGCGGCGACCGAGTTGCTCTTGCCCGGCGTCAATACGGGATAATACCGCG
CCACATAGCAGAACTTTAAAAGTGCTCATCATTGGAAAACGTTCTTCGGGGCGAAAACTCTCAAGGATCTTACCGC
TGTTGAGATCCAGTTCGATGTAACCCACTCGTGCACCCAACTGATCTTCAGCATCTTTTACTTTCACCAGCGTTTCT
GGGTGAGCAAAAACAGGAAGGCAAAATGCCGCAAAAAAGGGAATAAGGGCGACACGGAAATGTTGAATACTCAT
ACTCTTCCTTTTTCAATATTATTGAAGCATTTATCAGGGTTATTGTCTCATGAGCGGATACATATTTGAATGTATTTA
GAAAAATAAACAAATAGGGGTTCCGCGCACATTTCCCCGAAAAGTGCCACCTGACGTCTAAGAAACCATTATTATC
ATGACATTAACCTATAAAAATAGGCGTATCACGAGGCCCTTTCGTCTCGCGCGTTTCGGTGATGACGGTGAAAACC
TCTGACACATGCAGCTCCCGGAGACGGTCACAGCTTGTCTGTAAGCGGATGCCGGGAGCAGACAAGCCCGTCAGG
GCGCGTCAGCGGGTGTTGGCGGGTGTCGGGGCTGGCTTAACTATGCGGCATCAGAGCAGATTGTACTGAGAGTGC
ACCATATGCGGTGTGAAATACCGCACAGATGCGTAAGGAGAAAATACCGCATCAGGCGCCATTCGCCATTCAGGC
TGCGCAACTGTTGGGAAGGGCGATCGGTGCGGGCCTCTTCGCTATTACGCCAGCTGGCGAAAGGGGGATGTGCTGC
AAGGCGATTAAGTTGGGTAACGCCAGGGTTTTCCCAGTCACGACGTTGTAAAACGACGGCCAGTG

The plasmid pMU121 was deposited at the ATCC Patent Depository, 10801 University Blvd., Manassas, Va. 20110, on Sep. 10, 2008, as ATCC Deposit NO. ______. The present invention also encompasses a nucleic acid comprising a sequence that is at least about 70%, 75%, or 80% identical, preferably at least about 90% to about 95% identical, and more preferably at least about 96%, 97%, 98%, 99% or 100% identical to SEQ ID NO:10 or the plasmid deposited as ATCC Deposit No. ______.

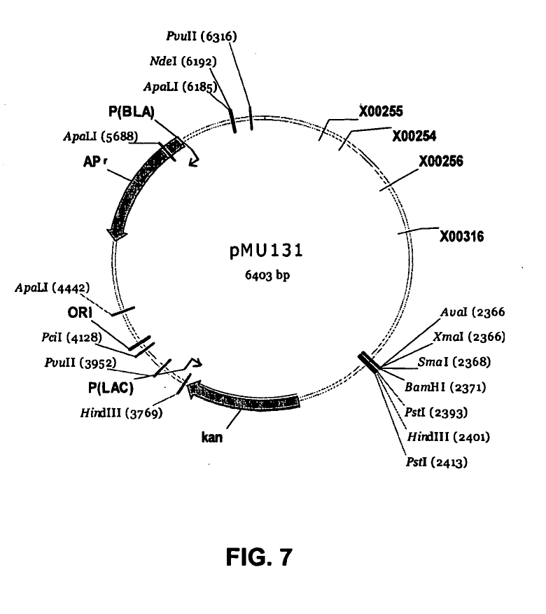

pMU131 has a size of about 6.4 kb and its map is shown in FIG. 7. The complete sequence of pMU131 is given in SEQ ID NO:11:

(SEQ ID NO: 11)
AATTGACAAAGTTTTCTATTTGTGTTAACATTGTTTATATAATAGTGAACAGTGTTAAGATTAAATGTGAGGTGTTT
GTATGGATATTAATGATTATAAAGAGAAGGGACTTTATTTATTAAGTAGTATGGATGATTTTATTAAAATTAATGAT
TTGTTTATGGGTAAAGTTGTTTCTCCTGGCTATGTTGCTTCGGTTTTTGGTGTTTCCAGGTCTACTGTTACACAATGG
ATTCAAAGACGTAAAATTAGAGCTTTTAAGTATAAAGGTAAGGAAGGTGACTATATGGTTATACCTATTGCTGATA
TTATTGATTACAAAAGATTGAGTAATAATGATTTTATTTATGATAAGTTAGTGAGGTGATTTATTTTATGTTTGACG
ATAGCTATGTTGTTAATGAGTGTTCGTCTAATGTTAGTGAAAATGATAGAGATTTTTGTAGTTTGGTTGGTCGTTTT
ATGATTATTAATGGTATAGATAAGTTGGTTATTAAGATTAATAGAAAATTTAATAGGAAATCTTTAAGTTTAGATTT
TAGTGTTGATTTATTCCCTTCTATCAAAGTTTCTGAATTAGTTTTTTTTGATGAGTTTAACAAAACGTGTGGTTTTTA
TTTTTCTTTTAATTCTTTTACAATTTTTAAGGCTTTTAGAGATGTTCATAATCATAATAAAATATCATTTTATTTTGCA
TAATTTCGGGTCTGGGCCGCAGACCAGGCCCAGTGCTAACAATATTAATTTTTAATGTTAGGAATTGTTTAATTCTT
AATTGTGTTTTTAAAGGTAGAATAATTACCCATTCGCCCTTTAGCCAACAAAAATTAAGGAGGTATAAACATGGAT
AAAATGGATTTGATTCTTCAAGATGAAAGACTGGGTGAGATATTTAAAGATATAGATTTAACAGATAATGAAAAG
AGATATCTTAAATGGTTATGGAAATGGGATTATGAAACACGTGATACTTTTGTATCAATTTTTTTGAAGCTAAAAAA
TGGTGGAAAATGATTTTTTTCTTATCTTGATATATTAGAAAAAAGCGTACTCACGAAGTAAGAATTTGTAAAAAAA
GAAGGGGGGATTTTTTTGGATGAGAGTTTGTACAAGCAGATTTTAAGTAATATTATTATTACTCGTGATTATTGTAA
AAATGTTTTAGATAATATAAAGTTCAATGAAAAAATAATTGATTATTATGTTATGTTACAAAATGATGTTTTTATTG
ATTTTACTAATAAAATAAATTCAATAAGGGATTGTAATAAATATTGGTATTTGGATGTTTATAAAAAGCAGAAAAT
AAAGGATTTTAAAAAGACTAATTTGTGTAAAGATAAGTTCTGTAATAATTGTAAGAAAGTTAAACAGGCTTCAAGA
ATGCAAAAATATATTCCTGAATTACAGAAATACAAAGATGGCTTATATCATTTTATATTTACTGTTGAAAATGTGCC
AGGTAGTGAATTAAGAGATACTATTGATAGGTTGTTTAAGTCTTTTAAGTCATTTACAAGGTATTTAAGTGGTAATC
TTAAAATAAAAGGTGTTAATTTTGATAAATGGGGTTATAAAGGCTGTGTAAGGTCTTTAGAGGTAACTTATAGTAT
GATTGATAATCATATTATGTATCATCCACACTTGCATGTTGCGATGATATTAGATCCTTTTTACGATGGTTTTAATGT
TGAAAGGATGCATATAATTAATAAGTTTAGTTATAGCTATGGTGTTTTAAAAAGGTTGTTTACTGATGATGAATTAT
TAATTCAAAAAATTTGGTATTTATTGTTTAATAATATTGAGGTTAACATGGCCAATATAAATAATTTAGAGGATGGT
TATTCTTGTTTAGTTAATAAGTTTAGTGATTATGATTATGCGGAGCTGTTTAAGTATATTTGTAAAAATACTGATGA
ACAAGGTTTACTTATGACTTATGATATTTTTAAAGATTTATATTTTGCATTACATAATGTTCATCAGATACAAGGCT
ATGGTTGTTTATATAATATAAGAGATGATACTCAATTAGATTTAAAGGTTGATGACATTTATAATGATTTGATTGAT
TTATTACAAGTTACAGAAAATCCTATACAGTCTATGGAAACTGTACAGGATTTATTAAAGGATACTGAATATACAA
TAATAAGCCGTAAGCGTATATTTAAGTATCTAACACAATTATATCATAAGGATTGATATTTATACCGTCTGTCGGAC
TCATGCGGAGGGGGACTTGAGGGGGTCTCCCCTCGCATTGTACGACAGACGGTATTATTATTATACAAATTTTTTTT
ATGTAATTTTTTTTGTGTAATTTTTTTATACAAATAATATTTCAATTCGAGCTCGGTACCCGGGGATCCTCTAGAGTC
GACCTGCAGGCATGCAACCTTGGCTGCAGGTCGATAAACCCAGCGAACCATTTGAGGTGATAGGTAAGATTATAC
CGAGGTATGAAAACGAGAATTGGACCTTTACAGAATTACTCTATGAAGCGCCATATTTAAAAAGCTACCAAGACG
AAGAGGATGAAGAGGATGAGGAGGCAGATTGCCTTGAATATATTGACAATACTGATAAGATAATATATCTTTTATA
TAGAAGATATCGCCGTATGTAAGGATTTCAGGGGGCAAGGCATAGGCAGCGCGCTTATCAATATATCTATAGAATG
GGCAAAGCATAAAAACTTGCATGGACTAATGCTTGAAACCCAGGACAATAACCTTATAGCTTGTAAATTCTATCAT
AATTGTGGTTTCAAAATCGGCTCCGTCGATACTATGTTATACGCCAACTTTCAAAACAACTTTGAAAAAGCTGTTTT
CTGGTATTTAAGGTTTTAGAATGCAAGGAACAGTGAATTGGAGTTCGTCTTGTTATAATTAGCTTCTTGGGGTATCT
TTAAATACTGTAGAAAAGAGGAAGGAAATAATAAATGGCTAAAATGAGAATATCACCGGAATTGAAAAAACTGAT
CGAAAAATACCGCTGCGTAAAAGATACGGAAGGAATGTCTCCTGCTAAGGTATATAAGCTGGTGGGAGAAAATGA
AAACCTATATTTAAAAATGACGGACAGCCGGTATAAAGGGACCACCTATGATGTGGAACGGGAAAAGGACATGAT
GCTATGGCTGGAAGGAAAGCTGCCTGTTCCAAAGGTCCTGCACTTTGAACGGCATGATGGCTGGAGCAATCTGCTC
ATGAGTGAGGCCGATGGCGTCCTTTGCTCGGAAGAGTATGAAGATGAACAAAGCCCTGAAAAGATTATCGAGCTG
TATGCGGAGTGCATCAGGCTCTTTCACTCCATCGACATATCGGATTGTCCCTATACGAATAGCTTAGACAGCCGCTT
AGCCGAATTGGATTACTTACTGAATAACGATCTGGCCGATGTGGATTGCGAAAACTGGGAAGAAGACACTCCATTT
AAAGATCCGCGCGAGCTGTATGATTTTTTAAAGACGGAAAAGCCCGAAGAGGAACTTGTCTTTTCCCACGGCGACC
TGGGAGACAGCAACATCTTTGTGAAAGATGGCAAAGTAAGTGGCTTTATTGATCTTGGGAGAAGCGGCAGGGCGG
ACAAGTGGTATGACATTGCCTTCTGCGTCCGGTCGATCAGGGAGGATATCGGGGAAGAACAGTATGTCGAGCTATT
TTTTGACTTACTGGGGATCAAGCCTGATTGGGAGAAAATAAAATATTATATTTTACTGGATGAATTGTTTTAGTACC
TAGATTTAGATGTCTAAAAAGCTTGGCGTAATCATGGTCATAGCTGTTTCCTGTGTGAAATTGTTATCCGCTCACAA
TTCCACACAACATACGAGCCGGAAGCATAAAGTGTAAAGCCTGGGGTGCCTAATGAGTGAGCTAACTCACATTAA
TTGCGTTGCGCTCACTGCCCGCTTTCCAGTCGGGAAACCTGTCGTGCCAGCTGCATTAATGAATCGGCCAACGCGC
GGGGAGAGGCGGTTTGCGTATTGGGCGCTCTTCCGCTTCCTCGCTCACTGACTCGCTGCGCTCGGTCGTTCGGCTGC
GGCGAGCGGTATCAGCTCACTCAAAGGCGGTAATACGGTTATCCACAGAATCAGGGGATAACGCAGGAAAGAACA
TGTGAGCAAAAGGCCAGCAAAAGGCCAGGAACCGTAAAAAGGCCGCGTTGCTGGCGTTTTTCCATAGGCTCCGCC
CCCCTGACGAGCATCACAAAAATCGACGCTCAAGTCAGAGGTGGCGAAACCCGACAGGACTATAAAGATACCAGG
CGTTTCCCCCTGGAAGCTCCCTCGTGCGCTCTCCTGTTCCGACCCTGCCGCTTACCGGATACCTGTCCGCCTTTCTCC
CTTCGGGAAGCGTGGCGCTTTCTCATAGCTCACGCTGTAGGTATCTCAGTTCGGTGTAGGTCGTTCGCTCCAAGCTG
GGCTGTGTGCACGAACCCCCCGTTCAGCCCGACCGCTGCGCCTTATCCGGTAACTATCGTCTTGAGTCCAACCCGGT
AAGACACGACTTATCGCCACTGGCAGCAGCCACTGGTAACAGGATTAGCAGAGCGAGGTATGTAGGCGGTGCTAC
AGAGTTCTTGAAGTGGTGGCCTAACTACGGCTACACTAGAAGGACAGTATTTGGTATCTGCGCTCTGCTGAAGCCA
GTTACCTTCGGAAAAAGAGTTGGTAGCTCTTGATCCGGCAAACAAACCACCGCTGGTAGCGGTGGTTTTTTTGTTTG
CAAGCAGCAGATTACGCGCAGAAAAAAAGGATCTCAAGAAGATCCTTTGATCTTTTCTACGGGGTCTGACGCTCAG
TGGAACGAAAACTCACGTTAAGGGATTTTGGTCATGAGATTATCAAAAAGGATCTTCACCTAGATCCTTTTAAATT
AAAAATGAAGTTTTAAATCAATCTAAAGTATATATGAGTAAACTTGGTCTGACAGTTACCAATGCTTAATCAGTGA
GGCACCTATCTCAGCGATCTGTCTATTTCGTTCATCCATAGTTGCCTGACTCCCCGTCGTGTAGATAACTACGATAC
GGGAGGGCTTACCATCTGGCCCCAGTGCTGCAATGATACCGCGAGACCCACGCTCACCGGCTCCAGATTTATCAGC
AATAAACCAGCCAGCCGGAAGGGCCGAGCGCAGAAGTGGTCCTGCAACTTTATCCGCCTCCATCCAGTCTATTAAT
TGTTGCCGGGAAGCTAGAGTAAGTAGTTCGCCAGTTAATAGTTTGCGCAACGTTGTTGCCATTGCTACAGGCATCG
TGGTGTCACGCTCGTCGTTTGGTATGGCTTCATTCAGCTCCGGTTCCCAACGATCAAGGCGAGTTACATGATCCCCC
ATGTTGTGCAAAAAAGCGGTTAGCTCCTTCGGTCCTCCGATCGTTGTCAGAAGTAAGTTGGCCGCAGTGTTATCACT
CATGGTTATGGCAGCACTGCATAATTCTCTTACTGTCATGCCATCCGTAAGATGCTTTTCTGTGACTGGTGAGTACT
CAACCAAGTCATTCTGAGAATAGTGTATGCGGCGACCGAGTTGCTCTTGCCCGGCGTCAATACGGGATAATACCGC
GCCACATAGCAGAACTTTAAAAGTGCTCATCATTGGAAAACGTTCTTCGGGGCGAAAACTCTCAAGGATCTTACCG
CTGTTGAGATCCAGTTCGATGTAACCCACTCGTGCACCCAACTGATCTTCAGCATCTTTTACTTTCACCAGCGTTTCT
GGGTGAGCAAAAACAGGAAGGCAAAATGCCGCAAAAAAGGGAATAAGGGCGACACGGAAATGTTGAATACTCAT
ACTCTTCCTTTTTCAATATTATTGAAGCATTTATCAGGGTTATTGTCTCATGAGCGGATACATATTTGAATGTATTTA
GAAAAATAAACAAATAGGGGTTCCGCGCACATTTCCCCGAAAAGTGCCACCTGACGTCTAAGAAACCATTATTATC
ATGACATTAACCTATAAAAATAGGCGTATCACGAGGCCCTTTCGTCTCGCGCGTTTCGGTGATGACGGTGAAAACC
TCTGACACATGCAGCTCCCGGAGACGGTCACAGCTTGTCTGTAAGCGGATGCCGGGAGCAGACAAGCCCGTCAGG
GCGCGTCAGCGGGTGTTGGCGGGTGTCGGGGCTGGCTTAACTATGCGGCATCAGAGCAGATTGTACTGAGAGTGC
ACCATATGCGGTGTGAAATACCGCACAGATGCGTAAGGAGAAAATACCGCATCAGGCGCCATTCGCCATTCAGGC
TGCGCAACTGTTGGGAAGGGCGATCGGTGCGGGCCTCTTCGCTATTACGCCAGCTGGCGAAAGGGGGATGTGCTGC
AAGGCGATTAAGTTGGGTAACGCCAGGGTTTTCCCAGTCACGACGTTGTAAAACGACGGCCAGTG

The plasmid pMU131 was deposited at the ATCC Patent Depository, 10801 University Blvd., Manassas, Va. 20110, on Sep. 10, 2008, as ATCC Deposit NO. ______. The present invention also encompasses a nucleic acid comprising a sequence that is at least about 70%, 75%, or 80% identical, preferably at least about 90% to about 95% identical, and more preferably at least about 96%, 97%, 98%, 99% or 100% identical to SEQ ID NO:11 or the plasmid deposited as ATCC Deposit No. ______.

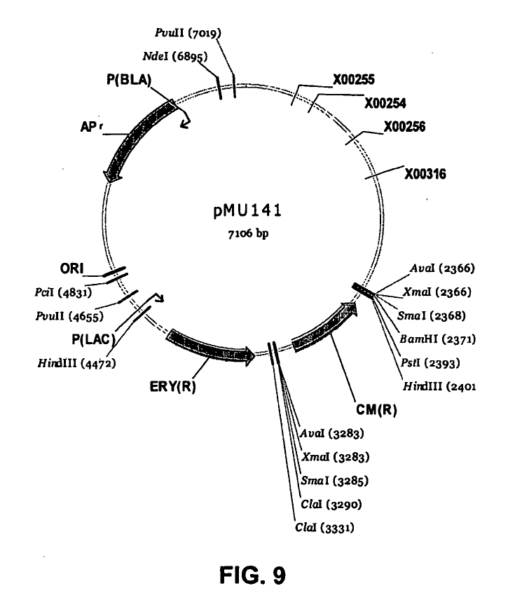

pMU141 has a size of about 7.1 kb and its map is shown in FIG. 9. The complete sequence of pMU141 is given in SEQ ID NO:14:

(SEQ ID NO: 14)
AATTGACAAAGTTTTCTATTTGTGTTAACATTGTTTATATAATAGTGAACAGTGTTAAGATTAAATGTGAGGTGTTT
GTATGGATATTAATGATTATAAAGAGAAGGGACTTTATTTATTAAGTAGTATGGATGATTTTATTAAAATTAATGAT
TTGTTTATGGGTAAAGTTGTTTCTCCTGGCTATGTTGCTTCGGTTTTTGGTGTTTCCAGGTCTACTGTTACACAATGG
ATTCAAAGACGTAAAATTAGAGCTTTTAAGTATAAAGGTAAGGAAGGTGACTATATGGTTATACCTATTGCTGATA
TTATTGATTACAAAAGATTGAGTAATAATGATTTTATTTATGATAAGTTAGTGAGGTGATTTATTTTATGTTTGACG
ATAGCTATGTTGTTAATGAGTGTTCGTCTAATGTTAGTGAAAATGATAGAGATTTTTGTAGTTTGGTTGGTCGTTTT
ATGATTATTAATGGTATAGATAAGTTGGTTATTAAGATTAATAGAAAATTTAATAGGAAATCTTTAAGTTTAGATTT
TAGTGTTGATTTATTCCCTTCTATCAAAGTTTCTGAATTAGTTTTTTTTGATGAGTTTAACAAAACGTGTGGTTTTTA
TTTTTCTTTTAATTCTTTTACAATTTTTAAGGCTTTTAGAGATGTTCATAATCATAATAAAATATCATTTTATTTTGCA
TAATTTCGGGTCTGGGCCGCAGACCAGGCCCAGTGCTAACAATATTAATTTTTAATGTTAGGAATTGTTTAATTCTT
AATTGTGTTTTTAAAGGTAGAATAATTACCCATTCGCCCTTTAGCCAACAAAAATTAAGGAGGTATAAACATGGAT
AAAATGGATTTGATTCTTCAAGATGAAAGACTGGGTGAGATATTTAAAGATATAGATTTAACAGATAATGAAAAG
AGATATCTTAAATGGTTATGGAAATGGGATTATGAAACACGTGATACTTTTGTATCAATTTTTTTGAAGCTAAAAAA
TGGTGGAAAATGATTTTTTTCTTATCTTGATATATTAGAAAAAAGCGTACTCACGAAGTAAGAATTTGTAAAAAAA
GAAGGGGGGATTTTTTTGGATGAGAGTTTGTACAAGCAGATTTTAAGTAATATTATTATTACTCGTGATTATTGTAA
AAATGTTTTAGATAATATAAAGTTCAATGAAAAAATAATTGATTATTATGTTATGTTACAAAATGATGTTTTTATTG
ATTTTACTAATAAAATAAATTCAATAAGGGATTGTAATAAATATTGGTATTTGGATGTTTATAAAAAGCAGAAAAT
AAAGGATTTTAAAAAGACTAATTTGTGTAAAGATAAGTTCTGTAATAATTGTAAGAAAGTTAAACAGGCTTCAAGA
ATGCAAAAATATATTCCTGAATTACAGAAATACAAAGATGGCTTATATCATTTTATATTTACTGTTGAAAATGTGCC
AGGTAGTGAATTAAGAGATACTATTGATAGGTTGTTTAAGTCTTTTAAGTCATTTACAAGGTATTTAAGTGGTAATC
TTAAAATAAAAGGTGTTAATTTTGATAAATGGGGTTATAAAGGCTGTGTAAGGTCTTTAGAGGTAACTTATAGTAT
GATTGATAATCATATTATGTATCATCCACACTTGCATGTTGCGATGATATTAGATCCTTTTTACGATGGTTTTAATGT
TGAAAGGATGCATATAATTAATAAGTTTAGTTATAGCTATGGTGTTTTAAAAAGGTTGTTTACTGATGATGAATTAT
TAATTCAAAAAATTTGGTATTTATTGTTTAATAATATTGAGGTTAACATGGCCAATATAAATAATTTAGAGGATGGT
TATTCTTGTTTAGTTAATAAGAAAGTGATTATGATTATGCGGAGCTGTTTAAGTATATTTGTAAAAATACTGATGA
ACAAGGTTTACTTATGACTTATGATATTTTTAAAGATTTATATTTTGCATTACATAATGTTCATCAGATACAAGGCT
ATGGTTATTTATATAATATAAGAGATGATACTCAATTAGATTTAAAGGTTGATGACATTTATAATGATTTGATTGAT
TTATTACAAGTTACAGAAAATCCTATACAGTCTATGGAAACTGTACAGGATTTATTAAAGGATACTGAATATACAA
TAATAAGCCGTAAGCGTATATTTAAGTATCTAACACAATTATATCATAAGGATTGATATTTATACCGTCTGTCGGAC
TCATGCGGAGGGGGACTTGAGGGGGTCTCCCCTCGCATTGTACGACAGACGGTATTATTATTATACAAATTTTTTTT
ATGTAATTTTTTTTGTGTAATTTTTTTATACAAATAATATTTCAATTCGAGCTCGGTACCCGGGGATCCTCTAGAGTC
GACCTGCAGGCATGCAAGCTTGTTATGTATAAAATTGTAGATTTTAGGGTAACAAAAAACACCGTATTTCTACGAT
GTTTTTGCTTAAATACTTGTTTTTAGTTACAGACAAACCTGAAGTTAACTATTTATCAATTCCTGCAATTCGTTTACA
AAACGGCAAATGTGAAATCCGTCACATACTGCGTGATGAACTTGAATTGCCAAAGGAAGTATAATTTTGTTATCTT
CTTTATAATATTTCCCCATAGTAAAAATAGGAATCAAATAATCATATCCTTTCTGCAAATTCAGATTAAAGCCATCG
AAGGTTGACCACGGTATCATAGATACATTAAAAATGTTTTCCGGAGCATTTGGCTTTCCTTCCATTCTATGATTGTT
TCCATACCGTTGCGTATCACTTTCATAATCTGCAAAAAATGATTTAAAGTCAGACTTACACTCAGTCCAAAGGCTGG
AAAATGTTTCAGTATCATTGTGAAATATTGTATAGCTTGGTATCATCTCATCATATATCCCCAATTCACCATCTTGA
TTGATTGCCGTCCTAAACTCTGAATGGCGGTTTACAATCATTGCAATATAATAAAGCATTGCAGGATATAGTTTCAT
TCCCTTTTCCTTTATTTGTGTGATATCCACTTTAACGGTCATGCTGTATGTACAAGGTACACTTGCAAAGTAGTGGTC
AAAATACTCTTTTCTGTTCCAACTATTTTTATCAATTTTTTCAAATACCATCTAAGTTCCCTCTCAAATTCAAGTTTA
TCGCTCTAATGAACAAAGATATTATACCACATTTTTGTGAATTTTTCAACTTGCCCACTTCGACTGCACTCCCGACT
TAATAACTTCTTGAACACTTGCCGAAAAAGAAAAACTGCCGGGTACGTACCCGGGATCGATCCCCGCCGAGCGCTT
AGTGGGAATTTGTACCCCTTATCGATACAAATTCCCCGTAGGCGCTAGGGACCTCTTTAGCTCCTTGGAAGCTGTCA
GTAGTATACCTAATAATTTATCTACATTCCCTTTAGTAACGTGTAACTTTCCAAATTTACAAAAGCGACTCATAGAA
TTATTTCCTCCCGTTAAATAATAGATAACTATTAAAAATAGACAATACTTGCTCATAAGTAACGGTACTTAAATTGT
TTACTTTGGCGTGTTTCATTGCTTGATGAAACTGATTTTTAGTAAACAGTTGACGATATTCTCGATTGACCCATTTTG
AAACAAAGTACGTATATAGCTTCCAATATTTATCTGGAACATCTGTGGTATGGCGGGTAAGTTTTATTAAGACACT
GTTTACTTTTGGTTTAGGATGAAAGCATTCCGCTGGCAGCTTAAGCAATTGCTGAATCGAGACTTGAGTGTGCAAG
AGCAACCCTAGTGTTCGGTGAATATCCAAGGTACGCTTGTAGAATCCTTCTTCAACAATCAGATAGATGTCAGACG
CATGGCTTTCAAAAACCACTTTTTTAATAATTTGTGTGCTTAAATGGTAAGGAATACTCCCAACAATTTTATACCTC
TGTTTGTTAGGGAATTGAAACTGTAGAATATCTTGGTGAATTAAAGTGACACGAGTATTCAGTTTTAATTTTTCTGA
CGATAAGTTGAATAGATGACTGTCTAATTCAATAGACGTTACCTGTTTACTTATTTTAGCCAGTTTCGTCGTTAAAT
GCCCTTTACCTGTTCCAATTTCGTAAACGGTATCGGTTTCTTTTAAATTCAATTGTTTTATTATTTGGTTGAGTACTTT
TTCACTCGTTAAAAAGTTTTGAGAATATTTTATATTTTTGTTCATGTAATCACTCCTCCTTAATTACAAATTAAAGC
ATCTAATTTAACTTCAATTCCTATTATACAAAATTTTAAGATACTGCACTATCAACACACTCTTAAGTTTGCTTCTAA
GTCTTATTTCCATAACTTCTTTTACGTTTCCGGGTACAATTCGTAATCATGTCATAGCTGTTTCCTGTGTGAAATTCT
TATCCGCTCACAATTCCACACAACATACGAGCCGGAAGCATAAAGTGTAAAGCCTGGGGTGCCTAATGAGTGAGC
TAACTCAAGCTTGGCGTAATCATGGTCATAGCTGTTTCCTGTGTGAAATTGTTATCCGCTCACAATTCCACACAACA
TACGAGCCGGAAGCATAAAGTGTAAAGCCTGGGGTGCCTAATGAGTGAGCTAACTCACATTAATTGCGTTGCGCTC
ACTGCCCGCTTTCCAGTCGGGAAACCTGTCGTGCCAGCTGCATTAATGAATCGGCCAACGCGCGGGGAGAGGCGGT
TTGCGTATTGGGCGCTCTTCCGCTTCCTCGCTCACTGACTCGCTGCGCTCGGTCGTTCGGCTGCGGCGAGCGGTATC
AGCTCACTCAAAGGCGGTAATACGGTTATCCACAGAATCAGGGGATAACGCAGGAAAGAACATGTGAGCAAAAG
GCCAGCAAAAGGCCAGGAACCGTAAAAAGGCCGCGTTGCTGGCGTTTTTCCATAGGCTCCGCCCCCCTGACGAGC
ATCACAAAAATCGACGCTCAAGTCAGAGGTGGCGAAACCCGACAGGACTATAAAGATACCAGGCGTTTCCCCCTG
GAAGCTCCCTCGTGCGCTCTCCTGTTCCGACCCTGCCGCTTACCGGATACCTGTCCGCCTTTCTCCCTTCGGGAAGC
GTGGCGCTTTCTCATAGCTCACGCTGTAGGTATCTCAGTTCGGTGTAGGTCGTTCGCTCCAAGCTGGGCTGTGTGCA
CGAACCCCCCGTTCAGCCCGACCGCTGCGCCTTATCCGGTAACTATCGTCTTGAGTCCAACCCGGTAAGACACGAC
TTATCGCCACTGGCAGCAGCCACTGGTAACAGGATTAGCAGAGCGAGGTATGTAGGCGGTGCTACAGAGTTCTTGA
AGTGGTGGCCTAACTACGGCTACACTAGAAGGACAGTATTTGGTATCTGCGCTCTGCTGAAGCCAGTTACCTTCGG
AAAAAGAGTTGGTAGCTCTTGATCCGGCAAACAAACCACCGCTGGTAGCGGTGGTTTTTTTGTTTGCAAGCAGCAG
ATTACGCGCAGAAAAAAAGGATCTCAAGAAGATCCTTTGATCTTTTCTACGGGGTCTGACGCTCAGTGGAACGAAA
ACTCACGTTAAGGGATTTTGGTCATGAGATTATCAAAAAGGATCTTCACCTAGATCCTTTTAAATTAAAAATGAAG
TTTTAAATCAATCTAAAGTATATATGAGTAAACTTGGTCTGACAGTTACCAATGCTTAATCAGTGAGGCACCTATCT
CAGCGATCTGTCTATTTCGTTCATCCATAGTTGCCTGACTCCCCGTCGTGTAGATAACTACGATACGGGAGGGCTTA
CCATCTGGCCCCAGTGCTGCAATGATACCGCGAGACCCACGCTCACCGGCTCCAGATTTATCAGCAATAAACCAGC
CAGCCGGAAGGGCCGAGCGCAGAAGTGGTCCTGCAACTTTATCCGCCTCCATCCAGTCTATTAATTGTTGCCGGGA
AGCTAGAGTAAGTAGTTCGCCAGTTAATAGAAGCGCAACGTTGTTGCCATTGCTACAGGCATCGTGGTGTCACGC
TCGTCGTTTGGTATGGCTTCATTCAGCTCCGGTTCCCAACGATCAAGGCGAGTTACATGATCCCCCATGTTGTGCAA
AAAAGCGGTTAGCTCCTTCGGTCCTCCGATCGTTGTCAGAAGTAAGTTGGCCGCAGTGTTATCACTCATGGTTATGG
CAGCACTGCATAATTCTCTTACTGTCATGCCATCCGTAAGATGCTTTTCTGTGACTGGTGAGTACTCAACCAAGTCA
TTCTGAGAATAGTGTATGCGGCGACCGAGTTGCTCTTGCCCGGCGTCAATACGGGATAATACCGCGCCACATAGCA
GAACTTTAAAAGTGCTCATCATTGGAAAACGTTCTTCGGGGCGAAAACTCTCAAGGATCTTACCGCTGTTGAGATC
CAGTTCGATGTAACCCACTCGTGCACCCAACTGATCTTCAGCATCTTTTACTTTCACCAGCGTTTCTGGGTGAGCAA
AAACAGGAAGGCAAAATGCCGCAAAAAAGGGAATAAGGGCGACACGGAAATGTTGAATACTCATACTCTTCCTTT
TTCAATATTATTGAAGCATTTATCAGGGTTATTGTCTCATGAGCGGATACATATTTGAATGTATTTAGAAAAATAAA
CAAATAGGGGTTCCGCGCACATTTCCCCGAAAAGTGCCACCTGACGTCTAAGAAACCATTATTATCATGACATTAA
CCTATAAAAATAGGCGTATCACGAGGCCCTTTCGTCTCGCGCGTTTCGGTGATGACGGTGAAAACCTCTGACACAT
GCAGCTCCCGGAGACGGTCACAGCTTGTCTGTAAGCGGATGCCGGGAGCAGACAAGCCCGTCAGGGCGCGTCAGC
GGGTGTTGGCGGGTGTCGGGGCTGGCTTAACTATGCGGCATCAGAGCAGATTGTACTGAGAGTGCACCATATGCGG
TGTGAAATACCGCACAGATGCGTAAGGAGAAAATACCGCATCAGGCGCCATTCGCCATTCAGGCTGCGCAACTGTT
GGGAAGGGCGATCGGTGCGGGCCTCTTCGCTATTACGCCAGCAAGCGAAAGGGGGATGTGCTGCAAGGCGATTAA
GTTGGGTAACGCCAGGGTTTTCCCAGTCACGACGTTGTAAAACGACGGCCAGTG

The present invention also encompasses a nucleic acid comprising a sequence that is at least about 70%, 75%, or 80% identical, preferably at least about 90% to about 95% identical, and more preferably at least about 96%, 97%, 98%, 99% or 100% identical to SEQ ID NO:14.

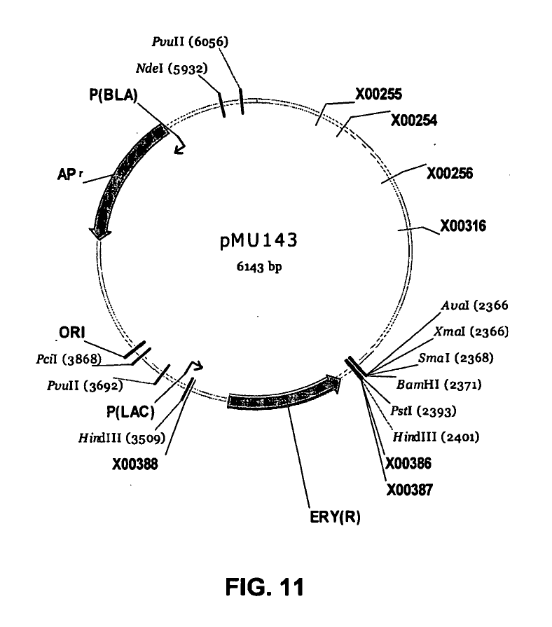

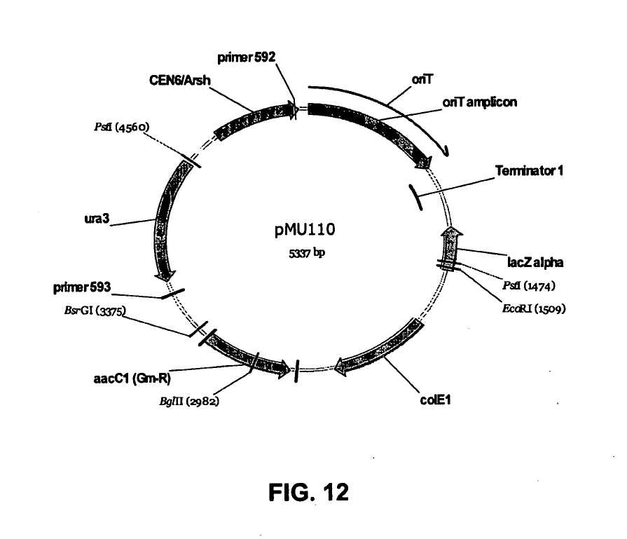

pMU143 has a size of about 6.1 kb and its map is shown in FIG. 11. The complete sequence of pMU143 is given in SEQ ID NO:17:

(SEQ ID NO: 17)
AATTGACAAAGTTTTCTATTTGTGTTAACATTGTTTATATAATAGTGAACAGTGTTAAGATTAAATGTGAGGTGTTT
GTATGGATATTAATGATTATAAAGAGAAGGGACTTTATTTATTAAGTAGTATGGATGATTTTATTAAAATTAATGAT
TTGTTTATGGGTAAAGTTGTTTCTCCTGGCTATGTTGCTTCGGAATTGGTGTTTCCAGGTCTACTGTTACACAATGG
ATTCAAAGACGTAAAATTAGAGCTTTTAAGTATAAAGGTAAGGAAGGTGACTATATGGTTATACCTATTGCTGATA
TTATTGATTACAAAAGATTGAGTAATAATGATTTTATTTATGATAAGTTAGTGAGGTGATTTATTTTATGTTTGACG
ATAGCTATGTTGTTAATGAGTGTTCGTCTAATGTTAGTGAAAATGATAGAGATTTTTGTAGTTTGGTTGGTCGTTTT
ATGATTATTAATGGTATAGATAAGTTGGTTATTAAGATTAATAGAAAATTTAATAGGAAATCTTTAAGTTTAGATTT
TAGTGTTGATTTATTCCCTTCTATCAAAGTTTCTGAATTAGTTTTTTTTGATGAGTTTAACAAAACGTGTGGTTTTTA
TTTTTCTTTTAATTCTTTTACAATTTTTAAGGCTTTTAGAGATGTTCATAATCATAATAAAATATCATTTTATTTTGCA
TAATTTCGGGTCTGGGCCGCAGACCAGGCCCAGTGCTAACAATATTAATTTTTAATGTTAGGAATTGTTTAATTCTT
AATTGTGTTTTTAAAGGTAGAATAATTACCCATTCGCCCTTTAGCCAACAAAAATTAAGGAGGTATAAACATGGAT
AAAATGGATTTGATTCTTCAAGATGAAAGACTGGGTGAGATATTTAAAGATATAGATTTAACAGATAATGAAAAG
AGATATCTTAAATGGTTATGGAAATGGGATTATGAAACACGTGATACATTTGTATCAATTTTTTTGAAGCTAAAAAA
TGGTGGAAAATGATTTTTTTCTTATCTTGATATATTAGAAAAAAGCGTACTCACGAAGTAAGAATTTGTAAAAAAA
GAAGGGGGGATTTTTTTGGATGAGAGTTTGTACAAGCAGATTTTAAGTAATATTATTATTACTCGTGATTATTGTAA
AAATGTTTTAGATAATATAAAGTTCAATGAAAAAATAATTGATTATTATGTTATGTTACAAAATGATGTTTTTATTG
ATTTTACTAATAAAATAAATTCAATAAGGGATTGTAATAAATATTGGTATTTGGATGTTTATAAAAAGCAGAAAAT
AAAGGATTTTAAAAAGACTAATTTGTGTAAAGATAAGTTCTGTAATAATTGTAAGAAAGTTAAACAGGCTTCAAGA
ATGCAAAAATATATTCCTGAATTACAGAAATACAAAGATGGCTTATATCATTTTATATTTACTGTTGAAAATGTGCC
AGGTAGTGAATTAAGAGATACTATTGATAGGTTGTTTAAGTCTTTTAAGTCATTTACAAGGTATTTAAGTGGTAATC
TTAAAATAAAAGGTGTTAATTTTGATAAATGGGGTTATAAAGGCTGTGTAAGGTCTTTAGAGGTAACTTATAGTAT
GATTGATAATCATATTATGTATCATCCACACTTGCATGTTGCGATGATATTAGATCCTTTTTACGATGGTTTTAATGT
TGAAAGGATGCATATAATTAATAAGTTTAGTTATAGCTATGGTGTTTTAAAAAGGTTGTTTACTGATGATGAATTAT
TAATTCAAAAAATTTGGTATTTATTGTTTAATAATATTGAGGTTAACATGGCCAATATAAATAATTTAGAGGATGGT
TATTCTTGTTTAGTTAATAAGTTTAGTGATTATGATTATGCGGAGCTGTTTAAGTATATTTGTAAAAATACTGATGA
ACAAGGTTTACTTATGACTTATGATATTTTTAAAGATTTATATTTTGCATTACATAATGTTCATCAGATACAAGGCT
ATGGTTGTTTATATAATATAAGAGATGATACTCAATTAGATTTAAAGGTTGATGACATTTATAATGATTTGATTGAT
TTATTACAAGTTACAGAAAATCCTATACAGTCTATGGAAACTGTACAGGATTTATTAAAGGATACTGAATATACAA
TAATAAGCCGTAAGCGTATATTTAAGTATCTAACACAATTATATCATAAGGATTGATATTTATACCGTCTGTCGGAC
TCATGCGGAGGGGGACTTGAGGGGGTCTCCCCTCGCATTGTACGACAGACGGTATTATTATTATACAAATTTTTTTT
ATGTAATTTTTTTTGTGTAATTTTTTTATACAAATAATATTTCAATTCGAGCTCGGTACCCGGGGATCCTCTAGAGTC
GACCTGCAGGCATGCAAGCTTGGTCTTTGTACTAACCTGTGGTTATGTATAAAATTGTAGATTTTAGGGTAACAAA
AAACACCGTATTTCTACGATGTTTTTGCTTAAATACTTGTTTTTAGTTACAGACAAACCTGAAGTTAACTATTTATCA
ATTCCTGCAATTCGTTTACAAAACGGCAAATGTGAAATCCGTCACATACTGCGTGATGAACTTGAATTGCCAAAGG
AAGTATAATTTTGTTATCTTCTTTATAATATTTCCCCATAGTAAAAATAGGAATCAAATAATCATATCCTTTCTGCA
AATTCAGATTAAAGCCATCGAAGGTTGACCACGGTATCATAGATACATTAAAAATGTTTTCCGGAGCATTTGGCTT
TCCTTCCATTCTATGATTGTTTCCATACCGTTGCGTATCACTTTCATAATCTGCTAAAAATGATTTAAAGTCAGACTT
ACACTCAGTCCAAAGGCTGGAAAATGTTTCAGTATCATTGTGAAATATTGTATAGCTTGGTATCATCTCATCATATA
TCCCCAATTCACCATCTTGATTGATTGCCGTCCTAAACTCTGAATGGCGGTTTACAATCATTGCAATATAATAAAGC
ATTGCAGGATATAGTTTCATTCCCTTTTCCTTTATTTGTGTGATATCCACTTTAACGGTCATGCTGTATGTACAAGGT
ACACTTGCAAAGTAGTGGTCAAAATACTCTTTTCTGTTCCAACTATTTTTATCAATTTTTTCAAATACCATCTAAGTT
CCCTCTCAAATTCAAGTTTATCGCTCTAATGAACAAAGATATTATACCACATTTTTGTGAATTTTTCAACTTGCCCA
CTTCGACTGCACTCCCGACTTAATAACTTCTTGAACACTTGCCGAAAAAGAAAAACTGCCGGGTACGTACCCGGGA
TCGATCCCCGCCGAGCGCTTAGTGGGAATTTGTACCCCTTATCGATACAAATTCCCCGTAGGCGCTAGGGACCTCTT
TAGCTCCTTGGAAGCTGTCAGTAGAAGCTTGGCGTAATCATGGTCATAGCTGTTTCCTGTGTGAAATTGTTATCCGC
TCACAATTCCACACAACATACGAGCCGGAAGCATAAAGTGTAAAGCCTGGGGTGCCTAATGAGTGAGCTAACTCA
CATTAATTGCGTTGCGCTCACTGCCCGCTTTCCAGTCGGGAAACCTGTCGTGCCAGCTGCATTAATGAATCGGCCAA
CGCGCGGGGAGAGGCGGTTTGCGTATTGGGCGCTCTTCCGCTTCCTCGCTCACTGACTCGCTGCGCTCGGTCGTTCG
GCTGCGGCGAGCGGTATCAGCTCACTCAAAGGCGGTAATACGGTTATCCACAGAATCAGGGGATAACGCAGGAAA
GAACATGTGAGCAAAAGGCCAGCAAAAGGCCAGGAACCGTAAAAAGGCCGCGTTGCTGGCGTTTTTCCATAGGCT
CCGCCCCCCTGACGAGCATCACAAAAATCGACGCTCAAGTCAGAGGTGGCGAAACCCGACAGGACTATAAAGATA
CCAGGCGTTTCCCCCTGGAAGCTCCCTCGTGCGCTCTCCTGTTCCGACCCTGCCGCTTACCGGATACCTGTCCGCCT
TTCTCCCTTCGGGAAGCGTGGCGCTTTCTCATAGCTCACGCTGTAGGTATCTCAGTTCGGTGTAGGTCGTTCGCTCC
AAGCTGGGCTGTGTGCACGAACCCCCCGTTCAGCCCGACCGCTGCGCCTTATCCGGTAACTATCGTCTTGAGTCCA
ACCCGGTAAGACACGACTTATCGCCACTGGCAGCAGCCACTGGTAACAGGATTAGCAGAGCGAGGTATGTAGGCG
GTGCTACAGAGTTCTTGAAGTGGTGGCCTAACTACGGCTACACTAGAAGGACAGTATTTGGTATCTGCGCTCTGCT
GAAGCCAGTTACCTTCGGAAAAAGAGTTGGTAGCTCTTGATCCGGCAAACAAACCACCGCTGGTAGCGGTGGTTTT
TTTGTTTGCAAGCAGCAGATTACGCGCAGAAAAAAAGGATCTCAAGAAGATCCTTTGATCTTTTCTACGGGGTCTG
ACGCTCAGTGGAACGAAAACTCACGTTAAGGGATTTTGGTCATGAGATTATCAAAAAGGATCTTCACCTAGATCCT
TTTAAATTAAAAATGAAGTTTTAAATCAATCTAAAGTATATATGAGTAAACTTGGTCTGACAGTTACCAATGCTTAA
TCAGTGAGGCACCTATCTCAGCGATCTGTCTATTTCGTTCATCCATAGTTGCCTGACTCCCCGTCGTGTAGATAACT
ACGATACGGGAGGGCTTACCATCTGGCCCCAGTGCTGCAATGATACCGCGAGACCCACGCTCACCGGCTCCAGATT
TATCAGCAATAAACCAGCCAGCCGGAAGGGCCGAGCGCAGAAGTGGTCCTGCAACTTTATCCGCCTCCATCCAGTC
TATTAATTGTTGCCGGGAAGCTAGAGTAAGTAGTTCGCCAGTTAATAGTTTGCGCAACGTTGTTGCCATTGCTACAG
GCATCGTGGTGTCACGCTCGTCGTTTGGTATGGCTTCATTCAGCTCCGAATCCCAACGATCAAGGCGAGTTACATGA
TCCCCCATGTTGTGCAAAAAAGCGGTTAGCTCCTTCGGTCCTCCGATCGTTGTCAGAAGTAAGTTGGCCGCAGTGTT
ATCACTCATGGTTATGGCAGCACTGCATAATTCTCTTACTGTCATGCCATCCGTAAGATGCTTTTCTGTGACTGGTG
AGTACTCAACCAAGTCATTCTGAGAATAGTGTATGCGGCGACCGAGTTGCTCTTGCCCGGCGTCAATACGGGATAA
TACCGCGCCACATAGCAGAACTTTAAAAGTGCTCATCATTGGAAAACGTTCTTCGGGGCGAAAACTCTCAAGGATC
TTACCGCTGTTGAGATCCAGTTCGATGTAACCCACTCGTGCACCCAACTGATCTTCAGCATCTTTTACTTTCACCAG
CGTTTCTGGGTGAGCAAAAACAGGAAGGCAAAATGCCGCAAAAAAGGGAATAAGGGCGACACGGAAATGTTGAA
TACTCATACTCTTCCTTTTTCAATATTATTGAAGCATTTATCAGGGTTATTGTCTCATGAGCGGATACATATTTGAAT
GTATTTAGAAAAATAAACAAATAGGGGTTCCGCGCACATTTCCCCGAAAAGTGCCACCTGACGTCTAAGAAACCAT
TATTATCATGACATTAACCTATAAAAATAGGCGTATCACGAGGCCCTTTCGTCTCGCGCGTTTCGGTGATGACGGTG
AAAACCTCTGACACATGCAGCTCCCGGAGACGGTCACAGCTTGTCTGTAAGCGGATGCCGGGAGCAGACAAGCCC
GTCAGGGCGCGTCAGCGGGTGTTGGCGGGTGTCGGGGCTGGCTTAACTATGCGGCATCAGAGCAGATTGTACTGA
GAGTGCACCATATGCGGTGTGAAATACCGCACAGATGCGTAAGGAGAAAATACCGCATCAGGCGCCATTCGCCAT
TCAGGCTGCGCAACTGTTGGGAAGGGCGATCGGTGCGGGCCTCTTCGCTATTACGCCAGCTGGCGAAAGGGGGAT
GTGCTGCAAGGCGATTAAGTTGGGTAACGCCAGGGTTTTCCCAGTCACGACGTTGTAAAACGACGGCCAGTG

The present invention also encompasses a nucleic acid comprising a sequence that is at least about 70%, 75%, or 80% identical, preferably at least about 90% to about 95% identical, and more preferably at least about 96%, 97%, 98%, 99% or 100% identical to SEQ ID NO:17.

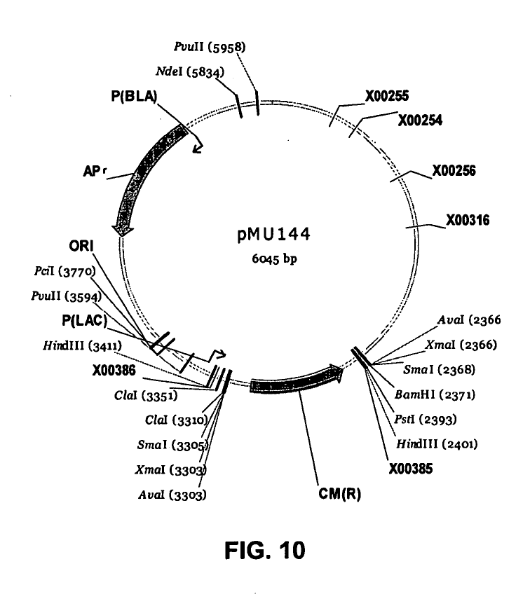

pMU144 has a size of about 6 kb and its map is shown in FIG. 10. The complete sequence of pMU144 is given in SEQ ID NO:20:

(SEQ ID NO: 20)
AATTGACAAAGTTTTCTATTTGTGTTAACATTGTTTATATAATAGTGAACAGTGTTAAGATTAAATGTGAGGTGTTT
GTATGGATATTAATGATTATAAAGAGAAGGGACTTTATTTATTAAGTAGTATGGATGATTTTATTAAAATTAATGAT
TTGTTTATGGGTAAAGTTGTTTCTCCTGGCTATGTTGCTTCGGTTTTTGGTGTTTCCAGGTCTACTGTTACACAATGG
ATTCAAAGACGTAAAATTAGAGCTTTTAAGTATAAAGGTAAGGAAGGTGACTATATGGTTATACCTATTGCTGATA
TTATTGATTACAAAAGATTGAGTAATAATGATTTTATTTATGATAAGTTAGTGAGGTGATTTATTTTATGTTTGACG
ATAGCTATGTTGTTAATGAGTGTTCGTCTAATGTTAGTGAAAATGATAGAGATTTTTGTAGTTTGGTTGGTCGTTTT
ATGATTATTAATGGTATAGATAAGTTGGTTATTAAGATTAATAGAAAATTTAATAGGAAATCTTTAAGTTTAGATTT
TAGTGTTGATTTATTCCCTTCTATCAAAGTTTCTGAATTAGTTTTTTTTGATGAGTTTAACAAAACGTGTGGTTTTTA
TTTTTCTTTTAATTCTTTACAATTTTTAAGGCTTTTAGAGATGTTCATAATCATAATAAAATATCATTTTATTTTGCA
TAATTTCGGGTCTGGGCCGCAGACCAGGCCCAGTGCTAACAATATTAATTTTTAATGTTAGGAATTGTTTAAATCTT
AATTGTGTTTTTAAAGGTAGAATAATTACCCATTCGCCCTTTAGCCAACAAAAATTAAGGAGGTATAAACATGGAT
AAAATGGATTTGATTCTTCAAGATGAAAGACTGGGTGAGATATTTAAAGATATAGATTTAACAGATAATGAAAAG
AGATATCTTAAATGGTTATGGAAATGGGATTATGAAACACGTGATACTTTTGTATCAATTTTTTTGAAGCTAAAAAA
TGGTGGAAAATGATTTTTTTCTTATCTTGATATATTAGAAAAAAGCGTACAAACGAAGTAAGAATTTGTAAAAAAA
GAAGGGGGGATTTTTTTGGATGAGAGTTTGTACAAGCAGATTTTAAGTAATATTATTATTACTCGTGATTATTGTAA
AAATGTTTTAGATAATATAAAGTTCAATGAAAAAATAATTGATTATTATGTTATGTTACAAAATGATGTTTTTATTG
ATTTTACTAATAAAATAAATTCAATAAGGGATTGTAATAAATATTGGTATTTGGATGTTTATAAAAAGCAGAAAAT
AAAGGATTTTAAAAAGACTAATTTGTGTAAAGATAAGTTCTGTAATAATTGTAAGAAAGTTAAACAGGCTTCAAGA
ATGCAAAAATATATTCCTGAATTACAGAAATACAAAGATGGCTTATATCATTTTTATTTACTGTTGAAAATGTGCC
AGGTAGTGAATTAAGAGATACTATTGATAGGTTGTTTAAGTCTTTTAAGTCATTTACAAGGTATTTAAGTGGTAATC
TTAAAATAAAAGGTGTTAATTTTGATAAATGGGGTTATAAAGGCTGTGTAAGGTCTTTAGAGGTAACTTATAGTAT
GATTGATAATCATATTATGTATCATCCACACTTGCATGTTGCGATGATATTAGATCCTTTTTACGATGGTTTTAATGT
TGAAAGGATGCATATAATTAATAAGTTTAGTTATAGCTATGGTGTTTTAAAAAGGTTGTTTACTGATGATGAATTAT
TAATTCAAAAAATTTGGTATTTATTGTTTAATAATATTGAGGTTAACATGGCCAATATAAATAATTTAGAGGATGGT
TATTCTTGTTTAGAAATAAGTTTAGTGATTATGATTATGCGGAGCTGTTTAAGTATATTTGTAAAAATACTGATGA
ACAAGGTTTACTTATGACTTATGATATTTTTAAAGATTTATATTTTGCATTACATAATGTTCATCAGATACAAGGCT
ATGGTTGTTTTATAATATAAGAGATGATACTCAATTAGATTTAAAGGTTGATGACATTTATAATGATTTGATTGAT
TTATTACAAGTTACAGAAAATCCTATACAGTCTATGGAAACTGTACAGGATTTATTAAAGGATACTGAATATACAA
TAATAAGCCGTAAGCGTATATTTAAGTATCTAACACAATTATATCATAAGGATTGATATTTATACCGTCTGTCGGAC
TCATGCGGAGGGGGACTTGAGGGGGTCTCCCCTCGCATTGTACGACAGACGGTATTATTATTATACAAATTTTTTTT
ATGTAATTTTTTTTGTGTAATTTTTTTATACAAATAATATTTCAATTCGAGCTCGGTACCCGGGGATCCTCTAGAGTC
GACCTGCAGGCATGCAAGCTTCTCCTTGGAAGCTGTCAGTAGTATACCTAATAATTTATCTACATTCCCTTTAGTAA
CGTGTAACTTTCCAAATTTACAAAAGCGACTCATAGAATTATTTCCTCCCGTTAAATAATAGATAACTATTAAAAAT
AGACAATACTTGCTCATAAGTAACGGTACTTAAATTGTTTACTTTGGCGTGTTTCATTGCTTGATGAAACTGATTTTT
AGTAAACAGTTGACGATATTCTCGATTGACCCATTTTGAAACAAAGTACGTATATAGCTTCCAATATTTATCTGGAA
CATCTGTGGTATGGCGGGTAAGTTTTATTAAGACACTGTTTACTTTTGGTTTAGGATGAAAGCATTCCGCTGGCAGC
TTAAGCAATTGCTGAATCGAGACTTGAGTGTGCAAGAGCAACCCTAGTGTTCGGTGAATATCCAAGGTACGCTTGT
AGAATCCTTCTTCAACAATCAGATAGATGTCAGACGCATGGCTTTCAAAAACCACTTTTTTAATAATTTGTGTGCTT
AAATGGTAAGGAATACTCCCAACAATTTTATACCTCTGTTTGTTAGGGAATTGAAACTGTAGAATATCTTGGTGAAT
TAAAGTGACACGAGTATTCAGTTTTAATTTTTCTGACGATAAGTTGAATAGATGACTGTCTAATTCAATAGACGTTA
CCTGTTTACTTATTTTAGCCAGTTTCGTCGTTAAATGCCCTTTACCTGTTCCAATTTCGTAAACGGTATCGGTTTCTT
TTAAATTCAATTGTTTTATTATTTGGTTGAGTACTTTTTCACTCGTTAAAAAGTTTTGAGAATAAATATATTTTTGTT
CATGTAATCACTCAACTTAATTACAAATTTTTAGCATCTAATTTAACTTCAATTCCTATTATACAAAATTTTAAGAT
ACTGCACTATCAACACACTCTTAAGTTTGCTTCTAAGTCTTATTTCCATAACTTCTTTTACGTTTCCGGGTACAAATC
GTAATCATGTCATAGCTGTTTCCTGTGTGAAATTCTTATCCGCTCACAATTCCACACAACATACGAGCCGGAAGCAT
AAAGTGTAAAGCCTGGGGTGCCTAATGAGTGAGCTAACTCAAGCTTGGCGTAATCATGGTCATAGCTGTTTCCTGT
GTGAAATTGTTATCCGCTCACAATTCCACACAACATACGAGCCGGAAGCATAAAGTGTAAAGCCTGGGGTGCCTAA
TGAGTGAGCTAACTCACATTAATTGCGTTGCGCTCACTGCCCGCTTTCCAGTCGGGAAACCTGTCGTGCCAGCTGCA
TTAATGAATCGGCCAACGCGCGGGGAGAGGCGGTTTGCGTATTGGGCGCTCTTCCGCTTCCTCGCTCACTGACTCG
CTGCGCTCGGTCGTTCGGCTGCGGCGAGCGGTATCAGCTCACTCAAAGGCGGTAATACGGTTATCCACAGAATCAG
GGGATAACGCAGGAAAGAACATGTGAGCAAAAGGCCAGCAAAAGGCCAGGAACCGTAAAAAGGCCGCGTTGCTG
GCGTTTTTCCATAGGCTCCGCCCCCCTGACGAGCATCACAAAAATCGACGCTCAAGTCAGAGGTGGCGAAACCCGA
CAGGACTATAAAGATACCAGGCGTTTCCCCCTGGAAGCTCCCTCGTGCGCTCTCCTGTTCCGACCCTGCCGCTTACC
GGATACCTGTCCGCCTTTCTCCCTTCGGGAAGCGTGGCGCTTTCTCATAGCTCACGCTGTAGGTATCTCAGTTCGGT
GTAGGTCGTTCGCTCCAAGCTGGGCTGTGTGCACGAACCCCCCGTTCAGCCCGACCGCTGCGCCTTATCCGGTAAC
TATCGTCTTGAGTCCAACCCGGTAAGACACGACTTATCGCCACTGGCAGCAGCCACTGGTAACAGGATTAGCAGAG
CGAGGTATGTAGGCGGTGCTACAGAGTTCTTGAAGTGGTGGCCTAACTACGGCTACACTAGAAGGACAGTATTTGG
TATCTGCGCTCTGCTGAAGCCAGTTACCTTCGGAAAAAGAGTTGGTAGCTCTTGATCCGGCAAACAAACCACCGCT
GGTAGCGGTGGTTTTTTTGTTTGCAAGCAGCAGATTACGCGCAGAAAAAAAGGATCTCAAGAAGATCCTTTGATCT
TTTCTACGGGGTCTGACGCTCAGTGGAACGAAAACTCACGTTAAGGGATTTTGGTCATGAGATTATCAAAAAGGAT
CTTCACCTAGATCCTTTTAAATTAAAAATGAAGTTTTAAATCAATCTAAAGTATATATGAGTAAACTTGGTCTGACA
GTTACCAATGCTTAATCAGTGAGGCACCTATCTCAGCGATCTGTCTATTTCGTTCATCCATAGTTGCCTGACTCCCC
GTCGTGTAGATAACTACGATACGGGAGGGCTTACCATCTGGCCCCAGTGCTGCAATGATACCGCGAGACCCACGCT
CACCGGCTCCAGATTTATCAGCAATAAACCAGCCAGCCGGAAGGGCCGAGCGCAGAAGTGGTCCTGCAACTTTAT
CCGCCTCCATCCAGTCTATTAATTGTTGCCGGGAAGCTAGAGTAAGTAGTTCGCCAGTTAATAGTTTGCGCAACGTT
GTTGCCATTGCTACAGGCATCGTGGTGTCACGCTCGTCGTTTGGTATGGCTTCATTCAGCTCCGGTTCCCAACGATC
AAGGCGAGTTACATGATCCCCCATGTTGTGCAAAAAAGCGGTTAGCTCCTTCGGTCCTCCGATCGTTGTCAGAAGT
AAGTTGGCCGCAGTGTTATCACTCATGGTTATGGCAGCACTGCATAATTCTAATACTGTCATGCCATCCGTAAGATG
CTTTTCTGTGACTGGTGAGTACTCAACCAAGTCATTCTGAGAATAGTGTATGCGGCGACCGAGTTGCTCTTGCCCGG
CGTCAATACGGGATAATACCGCGCCACATAGCAGAACTTTAAAAGTGCTCATCATTGGAAAACGTTCTTCGGGGCG
AAAACTCTCAAGGATCTTACCGCTGTTGAGATCCAGTTCGATGTAACCCACTCGTGCACCCAACTGATCTTCAGCAT
CTTTTACTTTCACCAGCGTTTCTGGGTGAGCAAAAACAGGAAGGCAAAATGCCGCAAAAAAGGGAATAAGGGCGA
CACGGAAATGTTGAATACTCATACTCTTCCTTTTTCAATATTATTGAAGCATTTATCAGGGTTATTGTCTCATGAGC
GGATACATATTTGAATGTATTTAGAAAAATAAACAAATAGGGGTTCCGCGCACATTTCCCCGAAAAGTGCCACCTG
ACGTCTAAGAAACCATTATTATCATGACATTAACCTATAAAAATAGGCGTATCACGAGGCCCTTTCGTCTCGCGCG
TTTCGGTGATGACGGTGAAAACCTCTGACACATGCAGCTCCCGGAGACGGTCACAGCTTGTCTGTAAGCGGATGCC
GGGAGCAGACAAGCCCGTCAGGGCGCGTCAGCGGGTGTTGGCGGGTGTCGGGGCTGGCTTAACTATGCGGCATCA
GAGCAGATTGTACTGAGAGTGCACCATATGCGGTGTGAAATACCGCACAGATGCGTAAGGAGAAAATACCGCATC
AGGCGCCATTCGCCATTCAGGCTGCGCAACTGTTGGGAAGGGCGATCGGTGCGGGCCTCTTCGCTATTACGCCAGC
TGGCGAAAGGGGGATGTGCTGCAAGGCGATTAAGTTGGGTAACGCCAGGGTTTTCCCAGTCACGACGTTGTAAAA
CGACGGCCAGTG

The present invention also encompasses a nucleic acid comprising a sequence that is at least about 70%, 75%, or 80% identical, preferably at least about 90% to about 95% identical, and more preferably at least about 96%, 97%, 98%, 99% or 100% identical to SEQ ID NO:20.

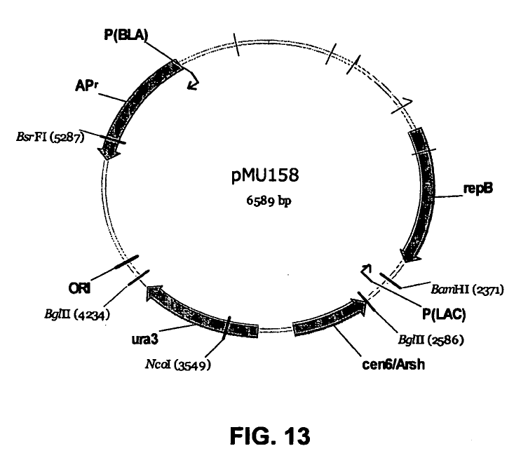

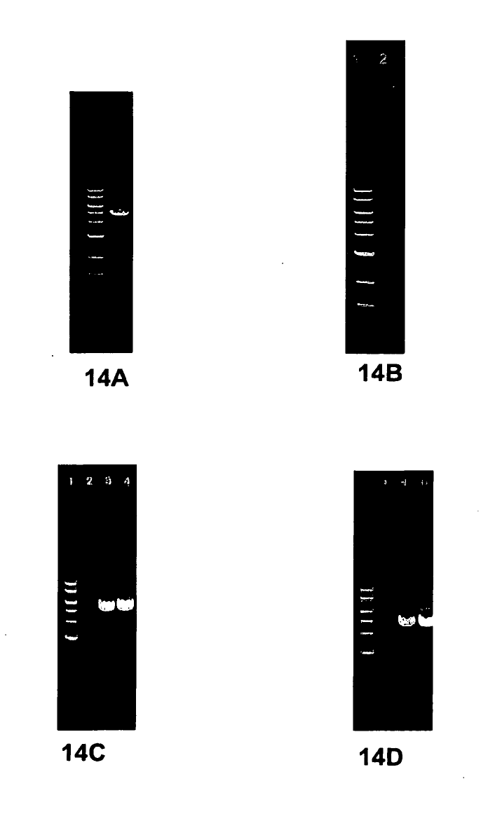

pMU158 has a size of about 6.5 kb and its map is shown in FIG. 13. The complete sequence of pMU158 is given in SEQ ID NO:25:

The present invention also encompasses a nucleic acid comprising a sequence that is at least about 70%, 75%, or 80% identical, preferably at least about 90% to about 95% identical, and more preferably at least about 96%, 97%, 98%, 99% or 100% identical to SEQ ID NO:25.

(SEQ ID NO: 25)
AATTGACAAAGTTTTCTATTTGTGTTAACATTGTTTATATAATAGTGAACAGTGTTAAGATTA
AATGTGAGGTGTTTGTATGGATATTAATGATTATAAAGAGAAGGGACTTTATTTATTAAGTA
GTATGGATGATTTTATTAAAATTAATGATTTGTTTATGGGTAAAGTTGTTTCTCCTGGCTATGT
TGCTTCGGTTTTTGGTGTTTCCAGGTCTACTGTTACACAATGGATTCAAAGACGTAAAATTAG
AGCTTTTAAGTATAAAGGTAAGGAAGGTGACTATATGGTTATACCTATTGCTGATATTATTGA
TTACAAAAGATTGAGTAATAATGATTTTATTTATGATAAGTTAGTGAGGTGATTTATTTTATG
TTTGACGATAGCTATGTTGTTAATGAGTGTTCGTCTAATGTTAGTGAAAATGATAGAGATTTT
TGTAGTTTGGTTGGTCGTTTTATGATTATTAATGGTATAGATAAGTTGGTTATTAAGATTAAT
AGAAAATTTAATAGGAAATCTTTAAGTTTAGATTTTAGTGTTGATTTATTCCCTTCTATCAAA
GTTTCTGAATTAGTTTTTTTTGATGAGTTTAACAAAACGTGTGGTTTTTATTTTTCTTTTAATTC
TTTTACAATTTTTAAGGCTTTTAGAGATGTTCATAATCATAATAAAATATCATTTTATTTTGCA
TAATTTCGGGTCTGGGCCGCAGACCAGGCCCAGTGCTAACAATATTAATTTTTAATGTTAGG
AATTGTTTAATTCTTAATTGTGTTTTTAAAGGTAGAATAATTACCCATTCGCCCTTTAGCCAA
CAAAAATTAAGGAGGTATAAACATGGATAAAATGGATTTGATTCTTCAAGATGAAAGACTG
GGTGAGATATTTAAAGATATAGATTTAACAGATAATGAAAAGAGATATCTTAAATGGTTATG
GAAATGGGATTATGAAACACGTGATACTTTTGTATCAATTTTTTTGAAGCTAAAAAATGGTG
GAAAATGATTTTTTTCTTATCTTGATATATTAGAAAAAAGCGTACTCACGAAGTAAGAATTTG
TAAAAAAAGAAGGGGGGATTTTTTTGGATGAGAGTTTGTACAAGCAGATTTTAAGTAATATT
ATTATTACTCGTGATTATTGTAAAAATGTTTTAGATAATATAAAGTTCAATGAAAAAATAATT
GATTATTATGTTATGTTACAAAATGATGTTTTTATTGATTTTACTAATAAAATAAATTCAATA
AGGGATTGTAATAAATATTGGTATTTGGATGTTTATAAAAAGCAGAAAATAAAGGATTTTAA
AAAGACTAATTTGTGTAAAGATAAGTTCTGTAATAATTGTAAGAAAGTTAAACAGGCTTCAA
GAATGCAAAAATATATTCCTGAATTACAGAAATACAAAGATGGCTTATATCATTTTATATTTA
CTGTTGAAAATGTGCCAGGTAGTGAATTAAGAGATACTATTGATAGGTTGTTTAAGTCTTTTA
AGTCATTTACAAGGTATTTAAGTGGTAATCTTAAAATAAAAGGTGTTAATTTTGATAAATGG
GGTTATAAAGGCTGTGTAAGGTCTTTAGAGGTAACTTATAGTATGATTGATAATCATATTATG
TATCATCCACACTTGCATGTTGCGATGATATTAGATCCTTTTTACGATGGTTTTAATGTTGAA
AGGATGCATATAATTAATAAGTTTAGTTATAGCTATGGTGTTTTAAAAAGGTTGTTTACTGAT
GATGAATTATTAATTCAAAAAATTTGGTATTTATTGTTTAATAATATTGAGGTTAACATGGCC
AATATAAATAATTTAGAGGATGGTTATTCTTGTTTAGTTAATAAGTTTAGTGATTATGATTAT
GCGGAGCTGTTTAAGTATATTTGTAAAAATACTGATGAACAAGGTTTACTTATGACTTATGAT
ATTTTTAAAGATTTATATTTTGCATTACATAATGTTCATCAGATACAAGGCTATGGTTGTTTAT
ATAATATAAGAGATGATACTCAATTAGATTTAAAGGTTGATGACATTTATAATGATTTGATTG
ATTTATTACAAGTTACAGAAAATCCTATACAGTCTATGGAAACTGTACAGGATTTATTAAAG
GATACTGAATATACAATAATAAGCCGTAAGCGTATATTTAAGTATCTAACACAATTATATCA
TAAGGATTGATATTTATACCGTCTGTCGGACTCATGCGGAGGGGGACTTGAGGGGGTCTCCC
CTCGCATTGTACGACAGACGGTATTATTATTATACAAATTTTTTTTATGTAATTTTTTTTGTGT
AATTTTTTTATACAAATAATATTTCAATTCGAGCTCGGTACCCGGGGATCCTCTAGAGTCGAC
CTGCAGGCATGCAAGCTTGGCGTAATCATGGTCATAGCTGTTTCCTGTGTGAAATTGTTATCC
GCTCACAATTCCACACAACATACGAGCCGGAAGCATAAAGTGTAAAGCCTGGGGTGCCTAAT
GAGTGAGCTAACTCACATTAATTGCGTTGCGCTCACTGCCCGCTTTCCAGTCGGGAAACCTGT
CGTGCCAGCAGATCTGATCGCTTGCCTGTAACTTACACGCGCCTCGTATCTTTTAATGATGGA
ATAATTTGGGAATTTACTCTGTGTTTATTTATTTTTATGTTTTGTATTTGGATTTTAGAAAGTA
AATAAAGAAGGTAGAAGAGTTACGGAATGAAGAAAAAAAAATAAACAAAGGTTTAAAAAA
TTTCAACAAAAAGCGTACTTTACATATATATTTATTAGACAAGAAAAGCAGATTAAATAGAT
ATACATTCGATTAACGATAAGTAAAATGTAAAATCACAGGATTTTCGTGTGTGGTCTTCTACA
CAGACAAGATGAAACAATTCGGCATTAATACCTGAGAGCAGGAAGAGCAAGATAAAAGGTA
GTATTTGTTGGCGATCCCCCTAGAGTCTTTTACATCTTCGGAAAACAAAAACTATTTTTTCTTT
AATTTCTTTTTTTACTTTCTATTTTTAATTTATATATTTATATTAAAAAATTTAAATTATAATTA
TTTTTATAGCACGTGATGAAAAGGACCCATCGATAAGCTAGCTTTTCAATTCAATTCATCATT
TTTTTTTTATTCTTTTTTTTGATTTCGGTTTCTTTGAAATTTTTTTGATTCGGTAATCTCCGAAC
AGAAGGAAGAACGAAGGAAGGAGCACAGACTTAGATTGGTATATATACGCATATGTAGTGT
TGAAGAAACATGAAATTGCCCAGTATTCTTAACCCAACTGCACAGAACAAAAACCTGCAGG
AAACGAAGATAAATCATGTCGAAAGCTACATATAAGGAACGTGCTGCTACTCATCCTAGTCC
TGTTGCTGCCAAGCTATTTAATATCATGCACGAAAAGCAAACAAACTTGTGTGCTTCATTGG
ATGTTCGTACCACCAAGGAATTACTGGAGTTAGTTGAAGCATTAGGTCCCAAAATTTGTTTAC
TAAAAACACATGTGGATATCTTGACTGATTTTTCCATGGAGGGCACAGTTAAGCCGCTAAAG
GCATTATCCGCCAAGTACAATTTTTTACTCTTCGAAGACAGAAAATTTGCTGACATTGGTAAT
ACAGTCAAATTGCAGTACTCTGCGGGTGTATACAGAATAGCAGAATGGGCAGACATTACGA
ATGCACACGGTGTGGTGGGCCCAGGTATTGTTAGCGGTTTGAAGCAGGCGGCAGAAGAAGT
AACAAAGGAACCTAGAGGCCTTTTGATGTTAGCAGAATTGTCATGCAAGGGCTCCCTATCTA
CTGGAGAATATACTAAGGGTACTGTTGACATTGCGAAGAGCGACAAAGATTTTGTTATCGGC
TTTATTGCTCAAAGAGACATGGGTGGAAGAGATGAAGGTTACGATTGGTTGATTATGACACC
CGGTGTGGGTTTAGATGACAAGGGAGACGCATTGGGTCAACAGTATAGAACCGTGGATGAT
GTGGTCTCTACAGGATCTGACATTATTATTGTTGGAAGAGGACTATTTGCAAAGGGAAGGGA
TGCTAAGGTAGAGGGTGAACGTTACAGAAAAGCAGGCTGGGAAGCATATTTGAGAAGATGC
GGCCAGCAAAACTAAAAAACTGTATTATAAGTAAATGCATGTATACTAAACTCACAAATTAG
AGCTTCAATTTAATTATATCAGTTATTACCCACTTTTCGAGATCTGCGGCGAGCGGTATCAGC
TCACTCAAAGGCGGTAATACGGTTATCCACAGAATCAGGGGATAACGCAGGAAAGAACATG
TGAGCAAAAGGCCAGCAAAAGGCCAGGAACCGTAAAAAGGCCGCGTTGCTGGCGTTTTTCC
ATAGGCTCCGCCCCCCTGACGAGCATCACAAAAATCGACGCTCAAGTCAGAGGTGGCGAAA
CCCGACAGGACTATAAAGATACCAGGCGTTTCCCCCTGGAAGCTCCCTCGTGCGCTCTCCTGT
TCCGACCCTGCCGCTTACCGGATACCTGTCCGCCTTTCTCCCTTCGGGAAGCGTGGCGCTTTC
TCATAGCTCACGCTGTAGGTATCTCAGTTCGGTGTAGGTCGTTCGCTCCAAGCTGGGCTGTGT
GCACGAACCCCCCGTTCAGCCCGACCGCTGCGCCTTATCCGGTAACTATCGTCTTGAGTCCAA
CCCGGTAAGACACGACTTATCGCCACTGGCAGCAGCCACTGGTAACAGGATTAGCAGAGCG
AGGTATGTAGGCGGTGCTACAGAGTTCTTGAAGTGGTGGCCTAACTACGGCTACACTAGAAG
GACAGTATTTGGTATCTGCGCTCTGCTGAAGCCAGTTACCTTCGGAAAAAGAGTTGGTAGCT
CTTGATCCGGCAAACAAACCACCGCTGGTAGCGGTGGTTTTTTTGTTTGCAAGCAGCAGATT
ACGCGCAGAAAAAAAGGATCTCAAGAAGATCCTTTGATCTTTTCTACGGGGTCTGACGCTCA
GTGGAACGAAAACTCACGTTAAGGGATTTTGGTCATGAGATTATCAAAAAGGATCTTCACCT
AGATCCTTTTAAATTAAAAATGAAGTTTTAAATCAATCTAAAGTATATATGAGTAAACTTGGT
CTGACAGTTACCAATGCTTAATCAGTGAGGCACCTATCTCAGCGATCTGTCTATTTCGTTCAT
CCATAGTTGCCTGACTCCCCGTCGTGTAGATAACTACGATACGGGAGGGCTTACCATCTGGC
CCCAGTGCTGCAATGATACCGCGAGACCCACGCTCACCGGCTCCAGATTTATCAGCAATAAA
CCAGCCAGCCGGAAGGGCCGAGCGCAGAAGTGGTCCTGCAACTTTATCCGCCTCCATCCAGT
CTATTAATTGTTGCCGGGAAGCTAGAGTAAGTAGTTCGCCAGTTAATAGTTTGCGCAACGTT
GTTGCCATTGCTACAGGCATCGTGGTGTCACGCTCGTCGTTTGGTATGGCTTCATTCAGCTCC
GGTTCCCAACGATCAAGGCGAGTTACATGATCCCCCATGTTGTGCAAAAAAGCGGTTAGCTC
CTTCGGTCCTCCGATCGTTGTCAGAAGTAAGTTGGCCGCAGTGTTATCACTCATGGTTATGGC
AGCACTGCATAATTCTCTTACTGTCATGCCATCCGTAAGATGCTTTTCTGTGACTGGTGAGTA
CTCAACCAAGTCATTCTGAGAATAGTGTATGCGGCGACCGAGTTGCTCTTGCCCGGCGTCAA
TACGGGATAATACCGCGCCACATAGCAGAACTTTAAAAGTGCTCATCATTGGAAAACGTTCT
TCGGGGCGAAAACTCTCAAGGATCTTACCGCTGTTGAGATCCAGTTCGATGTAACCCACTCG
TGCACCCAACTGATCTTCAGCATCTTTTACTTTCACCAGCGTTTCTGGGTGAGCAAAAACAGG
AAGGCAAAATGCCGCAAAAAAGGGAATAAGGGCGACACGGAAATGTTGAATACTCATACTC
TTCCTTTTTCAATATTATTGAAGCATTTATCAGGGTTATTGTCTCATGAGCGGATACATATTTG
AATGTATTTAGAAAAATAAACAAATAGGGGTTCCGCGCACATTTCCCCGAAAAGTGCCACCT
GACGTCTAAGAAACCATTATTATCATGACATTAACCTATAAAAATAGGCGTATCACGAGGCC
CTTTCGTCTCGCGCGTTTCGGTGATGACGGTGAAAACCTCTGACACATGCAGCTCCCGGAGA
CGGTCACAGCTTGTCTGTAAGCGGATGCCGGGAGCAGACAAGCCCGTCAGGGCGCGTCAGC
GGGTGTTGGCGGGTGTCGGGGCTGGCTTAACTATGCGGCATCAGAGCAGATTGTACTGAGAG
TGCACCATATGCGGTGTGAAATACCGCACAGATGCGTAAGGAGAAAATACCGCATCAGGCG
CCATTCGCCATTCAGGCTGCGCAACTGTTGGGAAGGGCGATCGGTGCGGGCCTCTTCGCTAT
TACGCCAGCTGGCGAAAGGGGGATGTGCTGCAAGGCGATTAAGTTGGGTAACGCCAGGGTT
TTCCCAGTCACGACGTTGTAAAACGACGGCCAGTG

pMU166 has a size of about 7 kb and its map is shown in FIG. 17. The complete sequence of pMU166 is given in SEQ ID NO:28.

The present invention also encompasses a nucleic acid comprising a sequence that is at least about 70%, 75%, or 80% identical, preferably at least about 90% to about 95% identical, and more preferably at least about 96%, 97%, 98%, 99% or 100% identical to SEQ ID NO:28.

(SEQ ID NO: 28)
AATTGACAAAGTTTTCTATTTGTGTTAACATTGTTTATATAATAGTGAACAGTGTTAAGATTA
AATGTGAGGTGTTTGTATGGATATTAATGATTATAAAGAGAAGGGACTTTATTTATTAAGTA
GTATGGATGATTTTATTAAAATTAATGATTTGTTTATGGGTAAAGTTGTTTCTCCTGGCTATGT
TGCTTCGGTTTTTGGTGTTTCCAGGTCTACTGTTACACAATGGATTCAAAGACGTAAAATTAG
AGCTTTTAAGTATAAAGGTAAGGAAGGTGACTATATGGTTATACCTATTGCTGATATTATTGA
TTACAAAAGATTGAGTAATAATGATTTTATTTATGATAAGTTAGTGAGGTGATTTATTTTATG
TTTGACGATAGCTATGTTGTTAATGAGTGTTCGTCTAATGTTAGTGAAAATGATAGAGATTTT
TGTAGTTTGGTTGGTCGTTTTATGATTATTAATGGTATAGATAAGTTGGTTATTAAGATTAAT
AGAAAATTTAATAGGAAATCTTTAAGTTTAGATTTTAGTGTTGATTTATTCCCTTCTATCAAA
GTTTCTGAATTAGTTTTTTTTGATGAGTTTAACAAAACGTGTGGTTTTTATTTTTCTTTTAATTC
TTTTACAATTTTTAAGGCTTTTAGAGATGTTCATAATCATAATAAAATATCATTTTATTTTGCA
TAATTTCGGGTCTGGGCCGCAGACCAGGCCCAGTGCTAACAATATTAATTTTTAATGTTAGG
AATTGTTTAATTCTTAATTGTGTTTTTAAAGGTAGAATAATTACCCATTCGCCCTTTAGCCAA
CAAAAATTAAGGAGGTATAAACATGGATAAAATGGATTTGATTCTTCAAGATGAAAGACTG
GGTGAGATATTTAAAGATATAGATTTAACAGATAATGAAAAGAGATATCTTAAATGGTTATG
GAAATGGGATTATGAAACACGTGATACTTTTGTATCAATTTTTTTGAAGCTAAAAAATGGTG
GAAAATGATTTTTTTCTTATCTTGATATATTAGAAAAAAGCGTACTCACGAAGTAAGAATTTG
TAAAAAAAGAAGGGGGGATTTTTTTGGATGAGAGTTTGTACAAGCAGATTTTAAGTAATATT
ATTATTACTCGTGATTATTGTAAAAATGTTTTAGATAATATAAAGTTCAATGAAAAAATAATT
GATTATTATGTTATGTTACAAAATGATGTTTTTATTGATTTTACTAATAAAATAAATTCAATA
AGGGATTGTAATAAATATTGGTATTTGGATGTTTATAAAAAGCAGAAAATAAAGGATTTTAA
AAAGACTAATTTGTGTAAAGATAAGTTCTGTAATAATTGTAAGAAAGTTAAACAGGCTTCAA
GAATGCAAAAATATATTCCTGAATTACAGAAATACAAAGATGGCTTATATCATTTTATATTTA
CTGTTGAAAATGTGCCAGGTAGTGAATTAAGAGATACTATTGATAGGTTGTTTAAGTCTTTTA
AGTCATTTACAAGGTATTTAAGTGGTAATCTTAAAATAAAAGGTGTTAATTTTGATAAATGG
GGTTATAAAGGCTGTGTAAGGTCTTTAGAGGTAACTTATAGTATGATTGATAATCATATTATG
TATCATCCACACTTGCATGTTGCGATGATATTAGATCCTTTTTACGATGGTTTTAATGTTGAA
AGGATGCATATAATTAATAAGTTTAGTTATAGCTATGGTGTTTTAAAAAGGTTGTTTACTGAT
GATGAATTATTAATTCAAAAAATTTGGTATTTATTGTTTAATAATATTGAGGTTAACATGGCC
AATATAAATAATTTAGAGGATGGTTATTCTTGTTTAGTTAATAAGTTTAGTGATTATGATTAT
GCGGAGCTGTTTAAGTATATTTGTAAAAATACTGATGAACAAGGTTTACTTATGACTTATGAT
ATTTTTAAAGATTTATATTTTGCATTACATAATGTTCATCAGATACAAGGCTATGGTTGTTTAT
ATAATATAAGAGATGATACTCAATTAGATTTAAAGGTTGATGACATTTATAATGATTTGATTG
ATTTATTACAAGTTACAGAAAATCCTATACAGTCTATGGAAACTGTACAGGATTTATTAAAG
GATACTGAATATACAATAATAAGCCGTAAGCGTATATTTAAGTATCTAACACAATTATATCA
TAAGGATTGATATTTATACCGTCTGTCGGACTCATGCGGAGGGGGACTTGAGGGGGTCTCCC
CTCGCATTGTACGACAGACGGTATTATTATTATACAAATTTTTTTTATGTAATTTTTTTTGTGT
AATTTTTTTATACAAATAATATTTCAATTCGAGCTCGGTACCCGGGGATCCTCTAGAGTCGAC
CTGCAGGCATGCAAGCTTGGCGTAATCATGGTCATAGCTGTTTCCTGTGTGAAATTGTTATCC
GCTCACAATTCCACACAACATACGAGCCGGAAGCATAAAGTGTAAAGCCTGGGGTGCCTAAT
GAGTGAGCTAACTCACATTAATTGCGTTGCGCTCACTGCCCGCTTTCCAGTCGGGAAACCTGT
CGTGCCAGCAGATCTGATCGCTTGCCTGTAACTTACACGCGCCTCGTATCTTTTAATGATGGA
ATAATTTGGGAATTTACTCTGTGTTTATTTATTTTTATGTTTTGTATTTGGATTTTAGAAAGTA
AATAAAGAAGGTAGAAGAGTTACGGAATGAAGAAAAAAAAATAAACAAAGGTTTAAAAAA
TTTCAACAAAAAGCGTACTTTACATATATATTTATTAGACAAGAAAAGCAGATTAAATAGAT
ATACATTCGATTAACGATAAGTAAAATGTAAAATCACAGGATTTTCGTGTGTGGTCTTCTACA
CAGACAAGATGAAACAATTCGGCATTAATACCTGAGAGCAGGAAGAGCAAGATAAAAGGTA
GTATTTGTTGGCGATCCCCCTAGAGTCTTTTACATCTTCGGAAAACAAAAACTATTTTTTCTTT
AATTTCTTTTTTTACTTTCTATTTTTAATTTATATATTTATATTAAAAAATTTAAATTATAATTA
TTTTTATAGCACGTGATGAAAAGGACCCATCGATAAGCTAGCTTTTCAATTCAATTCATCATT
TTTTTTTTATTCTTTTTTTTGATTTCGGTTTCTTTGAAATTTTTTTGATTCGGTAATCTCCGAAC
AGAAGGAAGAACGAAGGAAGGAGCACAGACTTAGATTGGTATATATACGCATATGTAGTGT
TGAAGAAACATGAAATTGCCCAGTATTCTTAACCCAACTGCACAGAACAAAAACCTGCAGG
AAACGAAGATAAATCATGTCGAAAGCTACATATAAGGAACGTGCTGCTACTCATCCTAGTCC
TGTTGCTGCCAAGCTATTTAATATCATGCACGAAAAGCAAACAAACTTGTGTGCTTCATTGG
ATGTTCGTACCACCAAGGAATTACTGGAGTTAGTTGAAGCATTAGGTCCCAAAATTTGTTTAC
TAAAAACACATGTGGATATCTTGACTGATTTTTCCATGGAGGGCACAGTTAAGCCGCTAAAG
GCATTATCCGCCAAGTACAATTTTTTACTCTTCGAAGACAGAAAATTTGCTGACATTGGTAAT
ACAGTCAAATTGCAGTACTCTGCGGGTGTATACAGAATAGCAGAATGGGCAGACATTACGA
ATGCACACGGTGTGGTGGGCCCAGGTATTGTTAGCGGTTTGAAGCAGGCGGCAGAAGAAGT
AACAAAGGAACCTAGAGGCCTTTTGATGTTAGCAGAATTGTCATGCAAGGGCTCCCTATCTA
CTGGAGAATATACTAAGGGTACTGTTGACATTGCGAAGAGCGACAAAGATTTTGTTATCGGC
TTTATTGCTCAAAGAGACATGGGTGGAAGAGATGAAGGTTACGATTGGTTGATTATGACACC
CGGTGTGGGTTTAGATGACAAGGGAGACGCATTGGGTCAACAGTATAGAACCGTGGATGAT
GTGGTCTCTACAGGATCTGACATTATTATTGTTGGAAGAGGACTATTTGCAAAGGGAAGGGA
TGCTAAGGTAGAGGGTGAACGTTACAGAAAAGCAGGCTGGGAAGCATATTTGAGAAGATGC
GGCCAGCAAAACTAAAAAACTGTATTATAAGTAAATGCATGTATACTAAACTCACAAATTAG
AGCTTCAATTTAATTATATCAGTTATTACCCACTTTTCGAGATCTGCGGCGAGCGGTATCAGC
TCACTCAAAGGCGGTAATACGGTTATCCACAGAATCAGGGGATAACGCAGGAAAGAACATG
TGAGCAAAAGGCCAGCAAAAGGCCAGGAACCGTAAAAAGGCCGCGTTGCTGGCGTTTTTCC
ATAGGCTCCGCCCCCCTGACGAGCATCACAAAAATCGACGCTCAAGTCAGAGGTGGCGAAA
CCCGACAGGACTATAAAGATACCAGGCGTTTCCCCCTGGAAGCTCCCTCGTGCGCTCTCCTGT
TCCGACCCTGCCGCTTACCGGATACCTGTCCGCCTTTCTCCCTTCGGGAAGCGTGGCGCTTTC
TCATAGCTCACGCTGTAGGTATCTCAGTTCGGTGTAGGTCGTTCGCTCCAAGCTGGGCTGTGT
GCACGAACCCCCCGTTCAGCCCGACCGCTGCGCCTTATCCGGTAACTATCGTCTTGAGTCCAA
CCCGGTAAGACACGACTTATCGCCACTGGCAGCAGCCACTGGTAACAGGATTAGCAGAGCG
AGGTATGTAGGCGGTGCTACAGAGTTCTTGAAGTGGTGGCCTAACTACGGCTACACTAGAAG
GACAGTATTTGGTATCTGCGCTCTGCTGAAGCCAGTTACCTTCGGAAAAAGAGTTGGTAGCT
CTTGATCCGGCAAACAAACCACCGCTGGTAGCGGTGGTTTTTTTGTTTGCAAGCAGCAGATT
ACGCGCAGAAAAAAAGGATCTCAAGAAGATCCTTTGATCTTTTCTACGGGGTCTGACGCTCA
GTGGAACGAAAACTCACGTTAAGGGATTTTGGTCATGAGATTATCAAAAAGGATCTTCACCT
AGATCCTTTTAAATTAAAAATGAAGTTTTAAATCAATCTAAAGTATATAGAGTCGATACAAA
TTCCTCGTAGGCGCTCGGGACCCCTATCTAGCGAACTTTTAGAAAAGATATAAAACATCAGA
GTATGGACAGTTGCGGATGTACTTCAGAAAAGATTAGATGTCTAAAAAGCTTTTTAGACATC
TAAATCTAGGTACTAAAACAATTCATCCAGTAAAATATAATATTTTATTTTCTCCCAATCAGG
CTTGATCCCCAGTAAGTCAAAAAATAGCTCGACATACTGTTCTTCCCCGATATCCTCCCTGAT
CGACCGGACGCAGAAGGCAATGTCATACCACTTGTCCGCCCTGCCGCTTCTCCCAAGATCAA
TAAAGCCACTTACTTTGCCATCTTTCACAAAGATGTTGCTGTCTCCCAGGTCGCCGTGGGAAA
AGACAAGTTCCTCTTCGGGCTTTTCCGTCTTTAAAAAATCATACAGCTCGCGCGGATCTTTAA
ATGGAGTGTCTTCTTCCCAGTTTTCGCAATCCACATCGGCCAGATCGTTATTCAGTAAGTAAT
CCAATTCGGCTAAGCGGCTGTCTAAGCTATTCGTATAGGGACAATCCGATATGTCGATGGAG
TGAAAGAGCCTGATGCACTCCGCATACAGCTCGATAATCTTTTCAGGGCTTTGTTCATCTTCA
TACTCTTCCGAGCAAAGGACGCCATCGGCCTCACTCATGAGCAGATTGCTCCAGCCATCATG
CCGTTCAAAGTGCAGGACCTTTGGAACAGGCAGCTTTCCTTCCAGCCATAGCATCATGTCCTT
TTCCCGTTCCACATCATAGGTGGTCCCTTTATACCGGCTGTCCGTCATTTTTAAATATAGGTTT
TCATTTTCTCCCACCAGCTTATATACCTTAGCAGGAGACATTCCTTCCGTATCTTTTACGCAGC
GGTATTTTTCGATCAGTTTTTTCAATTCCGGTGATATTCTCATTTTAGCCATTTATTATTTCCTT
CCTCTTTTCTACAGTATTTAAAGATACCCCAAGAAGCTAATTATAACAAGACGAACTCCAATT
CACTGTTCCTTGCATTCTAAAACCTTAAATACCAGAAAACAGCTTTTTCAAAGTTGTTTTGAA
AGTTGGCGTATAACATAGTATCGACGGAGCCGATTTTGAAACCACAATTATGATAGAATTTA
CAAGCTATAAGGTTATTGTCCTGGGTTTCAAGCATTAGTCCATGCAAGTTTTTATGCTTTGCC
CATTCTATAGATATATTGATAAGCGCGCTGCCTATGCCTTGCCCCCTGAAATCCTTACATACG
GCGATATCTTCTATATAAAAGATATATTATCTTATCAGTATTGTCAATATATTCAAGGCAATC
TGCCTCCTCATCCTCTTCATCCTCTTCGTCTTGGTAGCTTTTTAAATATGGCGCTTCATAGAGT
AATTCTGTAAAGGTCCAATTCTCGTTTTCATACCTCGGTATAATCTTACCTATCACCTCAAAT
GGTTCGCTGGGTTAAATAAACAAATAGGGGTTCCGCGCACATTTCCCCGAAAAGTGCCACCT
GACGTCTAAGAAACCATTATTATCATGACATTAACCTATAAAAATAGGCGTATCACGAGGCC
CTTTCGTCTCGCGCGTTTCGGTGATGACGGTGAAAACCTCTGACACATGCAGCTCCCGGAGA
CGGTCACAGCTTGTCTGTAAGCGGATGCCGGGAGCAGACAAGCCCGTCAGGGCGCGTCAGC
GGGTGTTGGCGGGTGTCGGGGCTGGCTTAACTATGCGGCATCAGAGCAGATTGTACTGAGAG
TGCACCATATGCGGTGTGAAATACCGCACAGATGCGTAAGGAGAAAATACCGCATCAGGCG
CCATTCGCCATTCAGGCTGCGCAACTGTTGGGAAGGGCGATCGGTGCGGGCCTCTTCGCTAT
TACGCCAGCTGGCGAAAGGGGGATGTGCTGCAAGGCGATTAAGTTGGGTAACGCCAGGGTT
TTCCCAGTCACGACGTTGTAAAACGACGGCCAGTG

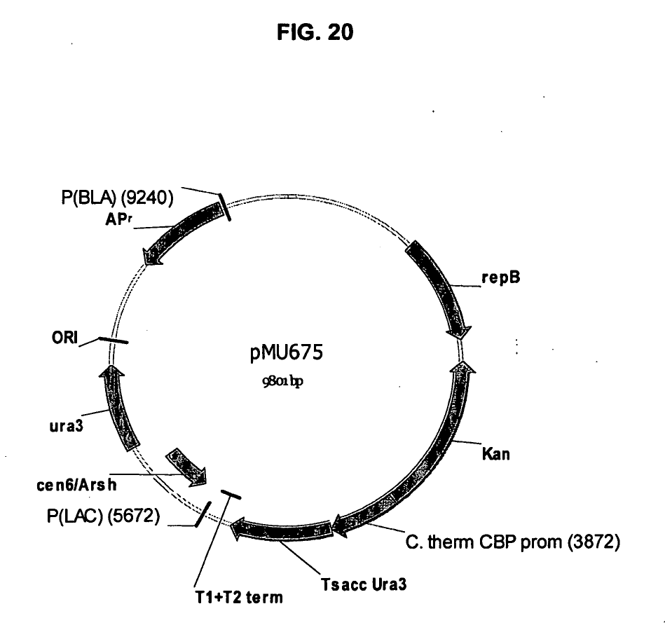

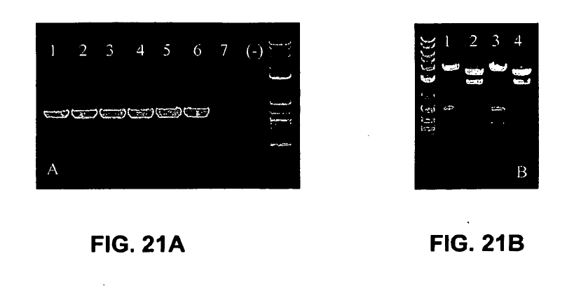

pMU675 has a size of about 9.8 kb and its map is shown in FIG. 20. The complete sequence of pMU675 is given in SEQ ID NO:39.

The present invention also encompasses a nucleic acid comprising a sequence that is at least about 70%, 75%, or 80% identical, preferably at least about 90% to about 95% identical, and more preferably at least about 96%, 97%, 98%, 99% or 100% identical to SEQ ID NO:39.

| LOCUS pMU675 9801 bp DNA circular | |
|---|---|
| FEATURES | Location/Qualifiers |
| rep_origin | 7586 . . . 7586 |
| | /vntifkey = “33” |
| | /label = ORI |
| | /note = “RNaseH cleavage point” |
| promoter | complement(5672 . . . 5672) |
| | /vntifkey = “30” |
| | /label = P(LAC) |
| | /note = “lac promoter” |
| CDS | complement(8348 . . . 9205) |
| | /vntifkey = “4” |
| | /label = AP(R) |
| | /note = “bla gene-Ap(r) determinant” |
| promoter | complement(9240 . . . 9240) |
| | /vntifkey = “30” |
| | /label = P(BLA) |
| | /note = “bla gene promoter” |
| CDS | 1207 . . . 2205 |
| | /vntifkey = “4” |
| | /label = repB |
| primer_bind | 9598 . . . 9618 |
| | /vntifkey = “28” |
| | /label = X00589 |
| CDS | 6555 . . . 7358 |
| | /vntifkey = “4” |
| | /label = ura3 |
| rep_origin | complement(5803 . . . 6316) |
| | /vntifkey = “33” |
| | /label = cen6/Arsh |
| CDS | 4493 . . . 5410 |
| | /vntifkey = “4” |
| | /label = Tsacc\Ura3 |
| CDS | complement(2401 . . . 3871) |
| | /vntifkey = “4” |
| | /label = Kan |
| terminator | 5411 . . . 5612 |
| | /vntifkey = “43” |
| | /label = T1 + T2\term |
| promoter | 3872 . . . 4492 |
| | /vntifkey = “30” |
| | /label = C.\therm\CBP\prom |

BASE COUNT 3017 a 1685 c 2051 g1 3048 t
ORIGIN

(SEQ ID NO: 39)

| | |
|---|---|
| 1 | aattgacaaa gttttctatt tgtgttaaca ttgtttatat aatagtgaac agtgttaaga |
| 61 | ttaaatgtga ggtgtttgta tggatattaa tgattataaa gagaagggac tttatttatt |
| 121 | aagtagtatg gatgatttta ttaaaattaa tgatttgttt atgggtaaag ttgtttctcc |
| 181 | tggctatgtt gcttcggttt ttggtgtttc caggtctact gttacacaat ggattcaaag |
| 241 | acgtaaaatt agagctttta agtataaagg taaggaaggt gactatatgg ttatacctat |
| 301 | tgctgatatt attgattaca aaagattgag taataatgat tttatttatg ataagttagt |
| 361 | gaggtgattt attttatgtt tgacgatagc tatgttgtta atgagtgttc gtctaatgtt |
| 421 | agtgaaaatg atagagattt ttgtagtttg gttggtcgtt ttatgattat taatggtata |
| 481 | gataagttgg ttattaagat taatagaaaa tttaatagga aatctttaag tttagatttt |
| 541 | agtgttgatt tattcccttc tatcaaagtt tctgaattag ttttttttga tgagtttaac |
| 601 | aaaacgtgtg gtttttattt ttcttttaat tcttttacaa tttttaaggc ttttagagat |
| 661 | gttcataatc ataataaaat atcattttat tttgcataat ttcgggtctg ggccgcagac |
| 721 | caggcccagt gctaacaata ttaattttta atgttaggaa ttgtttaatt cttaattgtg |
| 781 | tttttaaagg tagaataatt acccattcgc cctttagcca acaaaaatta aggaggtata |
| 841 | aacatggata aaatggattt gattcttcaa gatgaaagac tgggtgagat atttaaagat |
| 901 | atagatttaa cagataatga aaagagatat cttaaatggt tatggaaatg ggattatgaa |
| 961 | acacgtgata cttttgtatc aatttttttg aagctaaaaa atggtggaaa atgatttttt |
| 1021 | tcttatcttg atatattaga aaaaagcgta ctcacgaagt aagaatttgt aaaaaaagaa |
| 1081 | ggggggattt ttttggatga gagtttgtac aagcagattt taagtaatat tattattact |
| 1141 | cgtgattatt gtaaaaatgt tttagataat ataaagttca atgaaaaaat aattgattat |
| 1201 | tatgttatgt tacaaaatga tgtttttatt gattttacta ataaaataaa ttcaataagg |
| 1261 | gattgtaata aatattggta tttggatgtt tataaaaagc agaaaataaa ggattttaaa |
| 1321 | aagactaatt tgtgtaaaga taagttctgt aataattgta agaaagttaa acaggcttca |
| 1381 | agaatgcaaa aatatattcc tgaattacag aaatacaaag atggcttata tcattttata |
| 1441 | tttactgttg aaaatgtgcc aggtagtgaa ttaagagata ctattgatag gttgtttaag |
| 1501 | tcttttaagt catttacaag gtatttaagt ggtaatctta aaataaaagg tgttaatttt |
| 1561 | gataaatggg gttataaagg ctgtgtaagg tctttagagg taacttatag tatgattgat |
| 1621 | aatcatatta tgtatcatcc acacttgcat gttgcgatga tattagatcc tttttacgat |
| 1681 | ggttttaatg ttgaaaggat gcatataatt aataagttta gttatagcta tggtgtttta |
| 1741 | aaaaggttgt ttactgatga tgaattatta attcaaaaaa tttggtattt attgtttaat |
| 1801 | aatattgagg ttaacatggc caatataaat aatttagagg atggttattc ttgtttagtt |
| 1861 | aataagttta gtgattatga ttatgcggag ctgtttaagt atatttgtaa aaatactgat |
| 1921 | gaacaaggtt tacttatgac ttatgatatt tttaaagatt tatattttgc attacataat |
| 1981 | gttcatcaga tacaaggcta tggttgttta tataatataa gagatgatac tcaattagat |
| 2041 | ttaaaggttg atgacattta taatgatttg attgatttat tacaagttac agaaaatcct |
| 2101 | atacagtcta tggaaactgt acaggattta ttaaaggata ctgaatatac aataataagc |
| 2161 | cgtaagcgta tatttaagta tctaacacaa ttatatcata aggattgata tttataccgt |
| 2221 | ctgtcggact catgcggagg gggacttgag ggggtctccc ctcgcattgt acgacagacg |
| 2281 | gtattattat tatacaaatt ttttttatgt aatttttttt gtgtaatttt tttatacaaa |
| 2341 | taatatttca attcgagctc ggtacccggg gatcctctag agtcgacctg caggcatgca |
| 2401 | cgatacaaat tcctcgtagg cgctcgggac ccctatctag cgaactttta gaaaagatat |
| 2461 | aaaacatcag agtatggaca gttgcggatg tacttcagaa aagattagat gtctaaaaag |
| 2521 | ctttttagac atctaaatct aggtactaaa acaattcatc cagtaaaata taatatttta |
| 2581 | ttttctccca atcaggcttg atccccagta agtcaaaaaa tagctcgaca tactgttctt |
| 2641 | ccccgatatc ctccctgatc gaccggacgc agaaggcaat gtcataccac ttgtccgccc |
| 2701 | tgccgcttct cccaagatca ataaagccac ttactttgcc atctttcaca aagatgttgc |
| 2761 | tgtctcccag gtcgccgtgg gaaaagacaa gttcctcttc gggcttttcc gtctttaaaa |
| 2821 | aatcatacag ctcgcgcgga tctttaaatg gagtgtcttc ttcccagttt tcgcaatcca |
| 2881 | catcggccag atcgttattc agtaagtaat ccaattcggc taagcggctg tctaagctat |
| 2941 | tcgtataggg acaatccgat atgtcgatgg agtgaaagag cctgatgcac tccgcataca |
| 3001 | gctcgataat cttttcaggg ctttgttcat cttcatactc ttccgagcaa aggacgccat |
| 3061 | cggcctcact catgagcaga ttgctccagc catcatgccg ttcaaagtgc aggacctttg |
| 3121 | gaacaggcag ctttccttcc agccatagca tcatgtcctt ttcccgttcc acatcatagg |
| 3181 | tggtcccttt ataccggctg tccgtcattt ttaaatatag gttttcattt tctcccacca |
| 3241 | gcttatatac cttagcagga gacattcctt ccgtatcttt tacgcagcgg tatttttcga |
| 3301 | tcagtttttt caattccggt gatattctca ttttagccat ttattatttc cttcctcttt |
| 3361 | tctacagtat ttaaagatac cccaagaagc taattataac aagacgaact ccaattcact |
| 3421 | gttccttgca ttctaaaacc ttaaatacca gaaaacagct ttttcaaagt tgttttgaaa |
| 3481 | gttggcgtat aacatagtat cgacggagcc gattttgaaa ccacaattat gatagaattt |
| 3541 | acaagctata aggttattgt cctgggtttc aagcattagt ccatgcaagt ttttatgctt |
| 3601 | tgcccattct atagatatat tgataagcgc gctgcctatg ccttgccccc tgaaatcctt |
| 3661 | acatacggcg atatcttcta tataaaagat atattatctt atcagtattg tcaatatatt |
| 3721 | caaggcaatc tgcctcctca tcctcttcat cctcttcgtc ttggtagctt tttaaatatg |
| 3781 | gcgcttcata gagtaattct gtaaaggtcc aattctcgtt ttcatacctc ggtataatct |
| 3841 | tacctatcac ctcaaatggt tcgctgggtt tgagtcgtga ctaagaacgt caaagtaatt |
| 3901 | aacaatacag ctatttttct catgctttta cccctttcat aaaatttaat tttatcgtta |
| 3961 | tcataaaaaa ttatagacgt tatattgctt gccgggatat agtgctgggc attcgttggt |
| 4021 | gcaaaatgtt cggagtaagg tggatattga tttgcatgtt gatctattgc attgaaatga |
| 4081 | ttagttatcc gtaaatatta attaatcata tcataaatta attatatcat aattgttttg |
| 4141 | acgaatgaag gtttttggat aaattatcaa gtaaaggaac gctaaaaatt ttggcgtaaa |
| 4201 | atatcaaaat gaccacttga attaatatgg taaagtagat ataatatttt ggtaaacatg |
| 4261 | ccttcagcaa ggttagatta gctgtttccg tataaattaa ccgtatggta aaacggcagt |
| 4321 | cagaaaaata agtcataaga ttccgttatg aaaatatact tcggtagtta ataataagag |
| 4381 | atatgaggta agagatacaa gataagagat ataaggtacg aatgtataag atggtgcttt |
| 4441 | taggcacact aaataaaaaa caaataaacg aaaattttaa ggaggacgaa agatgttttc |
| 4501 | ggataatttg atacatgcaa taaaattcaa aaataatccc acggttgtcg gtttggatcc |
| 4561 | aagaattgaa agcattccag aattcataaa gaaagcggcc tttaataagt acgggaacaa |
| 4621 | tacaaaagga atatctgaag cgatgtataa ttttaataaa ggcattattg atgctgtatt |
| 4681 | tgatgtagta ccagcggtaa agattcaaat tgccttttac gaagtttatg gagcagatgg |
| 4741 | aatagaagct ttttataaaa ctgctgaata tgccaaagaa aaagggctta tagttatagc |
| 4801 | agatgtaaaa agaggtgata tagcagacgt agcagagatg tattcgaaag catatttgca |
| 4861 | gaatccatct attgacgcaa ttacaatcaa tccatacatg ggagaagata ccatgacacc |
| 4921 | atatatacat gacgtaatag aatacgataa aggactgttt attcttgtga aaacttccaa |
| 4981 | tgttggttct ggtacaattc aaaatttaaa aactatgaat ggcactgtgt atgaaaatgt |
| 5041 | ggcatacatg gttgataaga tttcaaaact ggccaaaggc agtttaggat atagttctat |
| 5101 | aggtgcagtt gttggagcta cgtataaaga ggaggccaaa atactgagaa aaataatgcc |
| 5161 | atctgctatc tttttggtgc ctggatatgg agcacagggt gctactgcag aagacgtcat |
| 5221 | taattgtttt gacgaaaaca acttaggtgc tatagttaac tcatcgagaa aagttatctt |
| 5281 | tgcttataaa agtcaatact ggaaagatgt ttattctgaa tatgagtatg ctcaagctgc |
| 5341 | acgtgctgaa gttcttctga tgatggggat gattaataat gcgtttttaa aaagaagata |
| 5401 | tgttgcgtgt taaaacgaaa ggctcagtcg aaagactggg cctttcgttt tatctgttgt |
| 5461 | ttgtcggtga acgctctcct gagtaggaca aatccgccgg gagcggattt gaacgttgcg |
| 5521 | aagcaacggc ccggagggtg gcgggcagga cgcccgccat aaactgccag gcatcaaatt |
| 5581 | aagcagaagg ccatcctgac ggatggcctt ttagcttggc gtaatcatgg tcatagctgt |
| 5641 | ttcctgtgtg aaattgttat ccgctcacaa ttccacacaa catacgagcc ggaagcataa |
| 5701 | agtgtaaagc ctggggtgcc taatgagtga gctaactcac attaattgcg ttgcgctcac |
| 5761 | tgcccgcttt ccagtcggga aacctgtcgt gccagcagat ctgatcgctt gcctgtaact |
| 5821 | tacacgcgcc tcgtatcttt taatgatgga ataatttggg aatttactct gtgtttattt |
| 5881 | atttttatgt tttgtatttg gattttagaa agtaaataaa gaaggtagaa gagttacgga |
| 5941 | atgaagaaaa aaaaataaac aaaggtttaa aaaatttcaa caaaaagcgt actttacata |
| 6001 | tatatttatt agacaagaaa agcagattaa atagatatac attcgattaa cgataagtaa |
| 6061 | aatgtaaaat cacaggattt tcgtgtgtgg tcttctacac agacaagatg aaacaattcg |
| 6121 | gcattaatac ctgagagcag gaagagcaag ataaaaggta gtatttgttg gcgatccccc |
| 6181 | tagagtcttt tacatcttcg gaaaacaaaa actatttttt ctttaatttc tttttttact |
| 6241 | ttctattttt aatttatata tttatattaa aaaatttaaa ttataattat ttttatagca |
| 6301 | cgtgatgaaa aggacccatc gataagctag cttttcaatt caattcatca tttttttttt |
| 6361 | attctttttt ttgatttcgg tttctttgaa atttttttga ttcggtaatc tccgaacaga |
| 6421 | aggaagaacg aaggaaggag cacagactta gattggtata tatacgcata tgtagtgttg |
| 6481 | aagaaacatg aaattgccca gtattcttaa cccaactgca cagaacaaaa acctgcagga |
| 6541 | aacgaagata aatcatgtcg aaagctacat ataaggaacg tgctgctact catcctagtc |
| 6601 | ctgttgctgc caagctattt aatatcatgc acgaaaagca aacaaacttg tgtgcttcat |
| 6661 | tggatgttcg taccaccaag gaattactgg agttagttga agcattaggt cccaaaattt |
| 6721 | gtttactaaa aacacatgtg gatatcttga ctgatttttc catggagggc acagttaagc |
| 6781 | cgctaaaggc attatccgcc aagtacaatt ttttactctt cgaagacaga aaatttgctg |
| 6841 | acattggtaa tacagtcaaa ttgcagtact ctgcgggtgt atacagaata gcagaatggg |
| 6901 | cagacattac gaatgcacac ggtgtggtgg gcccaggtat tgttagcggt ttgaagcagg |
| 6961 | cggcagaaga agtaacaaag gaacctagag gccttttgat gttagcagaa ttgtcatgca |
| 7021 | agggctccct atctactgga gaatatacta agggtactgt tgacattgcg aagagcgaca |
| 7081 | aagattttgt tatcggcttt attgctcaaa gagacatggg tggaagagat gaaggttacg |
| 7141 | attggttgat tatgacaccc ggtgtgggtt tagatgacaa gggagacgca ttgggtcaac |
| 7201 | agtatagaac cgtggatgat gtggtctcta caggatctga cattattatt gttggaagag |
| 7261 | gactatttgc aaagggaagg gatgctaagg tagagggtga acgttacaga aaagcaggct |
| 7321 | gggaagcata tttgagaaga tgcggccagc aaaactaaaa aactgtatta taagtaaatg |
| 7381 | catgtatact aaactcacaa attagagctt caatttaatt atatcagtta ttacccactt |
| 7441 | ttcgagatct gcggcgagcg gtatcagctc actcaaaggc ggtaatacgg ttatccacag |
| 7501 | aatcagggga taacgcagga aagaacatgt gagcaaaagg ccagcaaaag gccaggaacc |
| 7561 | gtaaaaaggc cgcgttgctg gcgtttttcc ataggctccg cccccctgac gagcatcaca |
| 7621 | aaaatcgacg ctcaagtcag aggtggcgaa acccgacagg actataaaga taccaggcgt |
| 7681 | ttccccctgg aagctccctc gtgcgctctc ctgttccgac cctgccgctt accggatacc |
| 7741 | tgtccgcctt tctcccttcg ggaagcgtgg cgctttctca tagctcacgc tgtaggtatc |
| 7801 | tcagttcggt gtaggtcgtt cgctccaagc tgggctgtgt gcacgaaccc cccgttcagc |
| 7861 | ccgaccgctg cgccttatcc ggtaactatc gtcttgagtc caacccggta agacacgact |
| 7921 | tatcgccact ggcagcagcc actggtaaca ggattagcag agcgaggtat gtaggcggtg |
| 7981 | ctacagagtt cttgaagtgg tggcctaact acggctacac tagaaggaca gtatttggta |
| 8041 | tctgcgctct gctgaagcca gttaccttcg gaaaaagagt tggtagctct tgatccggca |
| 8101 | aacaaaccac cgctggtagc ggtggttttt ttgtttgcaa gcagcagatt acgcgcagaa |
| 8161 | aaaaaggatc tcaagaagat cctttgatct tttctacggg gtctgacgct cagtggaacg |
| 8221 | aaaactcacg ttaagggatt ttggtcatga gattatcaaa aaggatcttc acctagatcc |
| 8281 | ttttaaatta aaaatgaagt tttaaatcaa tctaaagtat atatgagtaa acttggtctg |
| 8341 | acagttacca atgcttaatc agtgaggcac ctatctcagc gatctgtcta tttcgttcat |
| 8401 | ccatagttgc ctgactcccc gtcgtgtaga taactacgat acgggagggc ttaccatctg |
| 8461 | gccccagtgc tgcaatgata ccgcgagacc cacgctcacc ggctccagat ttatcagcaa |
| 8521 | taaaccagcc agccggaagg gccgagcgca gaagtggtcc tgcaacttta tccgcctcca |
| 8581 | tccagtctat taattgttgc cgggaagcta gagtaagtag ttcgccagtt aatagtttgc |
| 8641 | gcaacgttgt tgccattgct acaggcatcg tggtgtcacg ctcgtcgttt ggtatggctt |
| 8701 | cattcagctc cggttcccaa cgatcaaggc gagttacatg atcccccatg ttgtgcaaaa |
| 8761 | aagcggttag ctccttcggt cctccgatcg ttgtcagaag taagttggcc gcagtgttat |
| 8821 | cactcatggt tatggcagca ctgcataatt ctcttactgt catgccatcc gtaagatgct |
| 8881 | tttctgtgac tggtgagtac tcaaccaagt cattctgaga atagtgtatg cggcgaccga |
| 8941 | gttgctcttg cccggcgtca atacgggata ataccgcgcc acatagcaga actttaaaag |
| 9001 | tgctcatcat tggaaaacgt tcttcggggc gaaaactctc aaggatctta ccgctgttga |
| 9061 | gatccagttc gatgtaaccc actcgtgcac ccaactgatc ttcagcatct tttactttca |
| 9121 | ccagcgtttc tgggtgagca aaaacaggaa ggcaaaatgc cgcaaaaaag ggaataaggg |
| 9181 | cgacacggaa atgttgaata ctcatactct tcctttttca atattattga agcatttatc |
| 9241 | agggttattg tctcatgagc ggatacatat ttgaatgtat ttagaaaaat aaacaaatag |
| 9301 | gggttccgcg cacatttccc cgaaaagtgc cacctgacgt ctaagaaacc attattatca |
| 9361 | tgacattaac ctataaaaat aggcgtatca cgaggccctt tcgtctcgcg cgtttcggtg |
| 9421 | atgacggtga aaacctctga cacatgcagc tcccggagac ggtcacagct tgtctgtaag |
| 9481 | cggatgccgg gagcagacaa gcccgtcagg gcgcgtcagc gggtgttggc gggtgtcggg |
| 9541 | gctggcttaa ctatgcggca tcagagcaga ttgtactgag agtgcaccat atgcggtgtg |
| 9601 | aaataccgca cagatgcgta aggagaaaat accgcatcag gcgccattcg ccattcaggc |
| 9661 | tgcgcaactg ttgggaaggg cgatcggtgc gggcctcttc gctattacgc cagctggcga |
| 9721 | aagggggatg tgctgcaagg cgattaagtt gggtaacgcc agggttttcc cagtcacgac |
| 9781 | gttgtaaaac gacggccagt g |

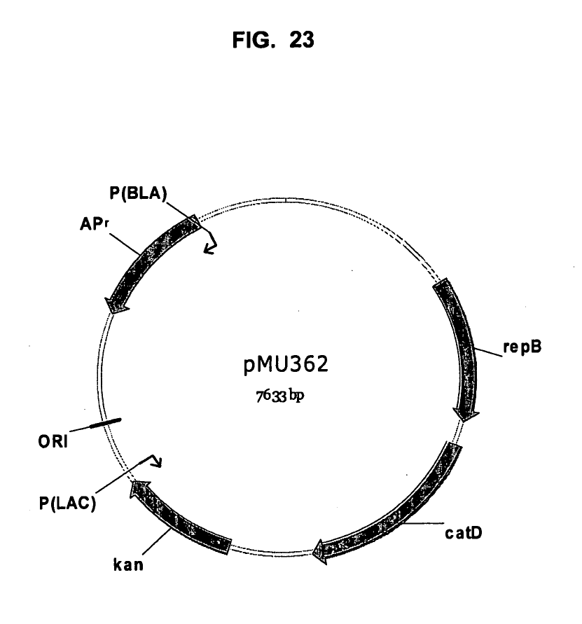

pMU362 has a size of about 7.6 kb and its map is shown in FIG. 23. The complete sequence of pMU166 is given in SEQ ID NO:40.

The present invention also encompasses a nucleic acid comprising a sequence that is at least about 70%, 75%, or 80% identical, preferably at least about 90% to about 95% identical, and more preferably at least about 96%, 97%, 98%, 99% or 100% identical to SEQ ID NO:40.

| LOCUS pMU362 7633 bp DNA circular | |
|---|---|
| FEATURES | Location/Qualifiers |
| rep_origin | 5418 . . . 5418 |
| | /vntifkey = “33” |
| | /label = ORI |
| | /note = “RNaseH cleavage point” |
| promoter | complement(5058 . . . 5058) |
| | /vntifkey = “30” |
| | /label = P(LAC) |
| | /note = “lac promoter” |
| CDS | complement(6180 . . . 7037) |
| | /vntifkey = “4” |
| | /label = AP(R) |
| | /note = “bla gene-Ap(r) determinant” |
| promoter | complement(7072 . . . 7072) |
| | /vntifkey = “30” |
| | /label = P(BLA) |
| | /note = “bla gene promoter” |
| CDS | 4181 . . . 4975 |
| | /vntifkey = “4” |
| | /label = kan |
| | /note = “kan from pMU131” |
| CDS | 2371 . . . 3623 |
| | /vntifkey = “4” |
| | /label = catD |
| CDS | 1207 . . . 2205 |
| | /vntifkey = “4” |
| | /label = repB |

| (SEQ ID NO: 40) | |
|---|---|
| 1 | aattgacaaa gttttctatt tgtgttaaca ttgtttatat aatagtgaac agtgttaaga |
| 61 | ttaaatgtga ggtgtttgta tggatattaa tgattataaa gagaagggac tttatttatt |
| 121 | aagtagtatg gatgatttta ttaaaattaa tgatttgttt atgggtaaag ttgtttctcc |
| 181 | tggctatgtt gcttcggttt ttggtgtttc caggtctact gttacacaat ggattcaaag |
| 241 | acgtaaaatt agagctttta agtataaagg taaggaaggt gactatatgg ttatacctat |
| 301 | tgctgatatt attgattaca aaagattgag taataatgat tttatttatg ataagttagt |
| 361 | gaggtgattt attttatgtt tgacgatagc tatgttgtta atgagtgttc gtctaatgtt |
| 421 | agtgaaaatg atagagattt ttgtagtttg gttggtcgtt ttatgattat taatggtata |
| 481 | gataagttgg ttattaagat taatagaaaa tttaatagga aatctttaag tttagatttt |
| 541 | agtgttgatt tattcccttc tatcaaagtt tctgaattag ttttttttga tgagtttaac |
| 601 | aaaacgtgtg gtttttattt ttcttttaat tcttttacaa tttttaaggc ttttagagat |
| 661 | gttcataatc ataataaaat atcattttat tttgcataat ttcgggtctg ggccgcagac |
| 721 | caggcccagt gctaacaata ttaattttta atgttaggaa ttgtttaatt cttaattgtg |
| 781 | tttttaaagg tagaataatt acccattcgc cctttagcca acaaaaatta aggaggtata |
| 841 | aacatggata aaatggattt gattcttcaa gatgaaagac tgggtgagat atttaaagat |
| 901 | atagatttaa cagataatga aaagagatat cttaaatggt tatggaaatg ggattatgaa |
| 961 | acacgtgata cttttgtatc aatttttttg aagctaaaaa atggtggaaa atgatttttt |
| 1021 | tcttatcttg atatattaga aaaaagcgta ctcacgaagt aagaatttgt aaaaaaagaa |
| 1081 | ggggggattt ttttggatga gagtttgtac aagcagattt taagtaatat tattattact |
| 1141 | cgtgattatt gtaaaaatgt tttagataat ataaagttca atgaaaaaat aattgattat |
| 1201 | tatgttatgt tacaaaatga tgtttttatt gattttacta ataaaataaa ttcaataagg |
| 1261 | gattgtaata aatattggta tttggatgtt tataaaaagc agaaaataaa ggattttaaa |
| 1321 | aagactaatt tgtgtaaaga taagttctgt aataattgta agaaagttaa acaggcttca |
| 1381 | agaatgcaaa aatatattcc tgaattacag aaatacaaag atggcttata tcattttata |
| 1441 | tttactgttg aaaatgtgcc aggtagtgaa ttaagagata ctattgatag gttgtttaag |
| 1501 | tcttttaagt catttacaag gtatttaagt ggtaatctta aaataaaagg tgttaatttt |
| 1561 | gataaatggg gttataaagg ctgtgtaagg tctttagagg taacttatag tatgattgat |
| 1621 | aatcatatta tgtatcatcc acacttgcat gttgcgatga tattagatcc tttttacgat |
| 1681 | ggttttaatg ttgaaaggat gcatataatt aataagttta gttatagcta tggtgtttta |
| 1741 | aaaaggttgt ttactgatga tgaattatta attcaaaaaa tttggtattt attgtttaat |
| 1801 | aatattgagg ttaacatggc caatataaat aatttagagg atggttattc ttgtttagtt |
| 1861 | aataagttta gtgattatga ttatgcggag ctgtttaagt atatttgtaa aaatactgat |
| 1921 | gaacaaggtt tacttatgac ttatgatatt tttaaagatt tatattttgc attacataat |
| 1981 | gttcatcaga tacaaggcta tggttgttta tataatataa gagatgatac tcaattagat |
| 2041 | ttaaaggttg atgacattta taatgatttg attgatttat tacaagttac agaaaatcct |
| 2101 | atacagtcta tggaaactgt acaggattta ttaaaggata ctgaatatac aataataagc |
| 2161 | cgtaagcgta tatttaagta tctaacacaa ttatatcata aggattgata tttataccgt |
| 2221 | ctgtcggact catgcggagg gggacttgag ggggtctccc ctcgcattgt acgacagacg |
| 2281 | gtattattat tatacaaatt ttttttatgt aatttttttt gtgtaatttt tttatacaaa |
| 2341 | taatatttca attcgagctc ggtacccggg atatggatcc agcttccaag gagctaaaga |
| 2401 | ggtccctagc gcctacgggg aatttgtatc gataaggggt acaaattccc actaagcgct |
| 2461 | cggcggggat cgatcccggg tacgtacccg gcagtttttc tttttcggca agtgttcaag |
| 2521 | aagttattaa gtcgggagtg cagtcgaagt gggcaagttg aaaaattcac aaaaatgtgg |
| 2581 | tataatatct ttgttcatta gagcgataaa cttgaatttg agagggaact tagatggtat |
| 2641 | ttgaaaaaat tgataaaaat agttggaaca gaaaagagta ttttgaccac tactttgcaa |
| 2701 | gtgtaccttg tacatacagc atgaccgtta aagtggatat cacacaaata aaggaaaagg |
| 2761 | gaatgaaact atatcctgca atgctttatt atattgcaat gattgtaaac cgccattcag |
| 2821 | agtttaggac ggcaatcaat caagatggtg aattggggat atatgatgag atgataccaa |
| 2881 | gctatacaat atttcacaat gatactgaaa cattttccag cctttggact gagtgtaagt |
| 2941 | ctgactttaa atcattttta gcagattatg aaagtgatac gcaacggtat ggaaacaatc |
| 3001 | atagaatgga aggaaagcca aatgctccgg aaaacatttt taatgtatct atgataccgt |
| 3061 | ggtcaacctt cgatggcttt aatctgaatt tgcagaaagg atatgattat ttgattccta |
| 3121 | tttttactat ggggaaatat tataaagaag ataacaaaat tatacttcct ttggcaattc |
| 3181 | aagttcatca cgcagtatgt gacggatttc acatttgccg ttttgtaaac gaattgcagg |
| 3241 | aattgataaa tagttaactt caggtttgtc tgtaactaaa aacaagtatt taagcaaaaa |
| 3301 | catcgtagaa atacggtgtt ttttgttacc ctaaaatcta caattttata cataaccaca |
| 3361 | ggttagtaca aagaccttgt gtttcttttt gaaaggctta aaacaaggat ttttccttga |
| 3421 | tttaagcccc gaaaagcaac acaaccaagg ttttagtatc aatctgtggt ttttatattt |
| 3481 | tcagagaaaa ggagaacaag aaaaaatgaa actaaatgaa aacgaaatga atttcagcgt |
| 3541 | acctcttgaa atcatcaagg caagtgaaat cgagccgaaa gaagtaaagt ggctgtggta |
| 3601 | tccgtatatt ccgctgcaga tatgcatgca agcttggctg caggtcgata aacccagcga |
| 3661 | accatttgag gtgataggta agattatacc gaggtatgaa aacgagaatt ggacctttac |
| 3721 | agaattactc tatgaagcgc catatttaaa aagctaccaa gacgaagagg atgaagagga |
| 3781 | tgaggaggca gattgccttg aatatattga caatactgat aagataatat atcttttata |
| 3841 | tagaagatat cgccgtatgt aaggatttca gggggcaagg cataggcagc gcgcttatca |
| 3901 | atatatctat agaatgggca aagcataaaa acttgcatgg actaatgctt gaaacccagg |
| 3961 | acaataacct tatagcttgt aaattctatc ataattgtgg tttcaaaatc ggctccgtcg |
| 4021 | atactatgtt atacgccaac tttcaaaaca actttgaaaa agctgttttc tggtatttaa |
| 4081 | ggttttagaa tgcaaggaac agtgaattgg agttcgtctt gttataatta gcttcttggg |
| 4141 | gtatctttaa atactgtaga aaagaggaag gaaataataa atggctaaaa tgagaatatc |
| 4201 | accggaattg aaaaaactga tcgaaaaata ccgctgcgta aaagatacgg aaggaatgtc |
| 4261 | tcctgctaag gtatataagc tggtgggaga aaatgaaaac ctatatttaa aaatgacgga |
| 4321 | cagccggtat aaagggacca cctatgatgt ggaacgggaa aaggacatga tgctatggct |
| 4381 | ggaaggaaag ctgcctgttc caaaggtcct gcactttgaa cggcatgatg gctggagcaa |
| 4441 | tctgctcatg agtgaggccg atggcgtcct ttgctcggaa gagtatgaag atgaacaaag |
| 4501 | ccctgaaaag attatcgagc tgtatgcgga gtgcatcagg ctctttcact ccatcgacat |
| 4561 | atcggattgt ccctatacga atagcttaga cagccgctta gccgaattgg attacttact |
| 4621 | gaataacgat ctggccgatg tggattgcga aaactgggaa gaagacactc catttaaaga |
| 4681 | tccgcgcgag ctgtatgatt ttttaaagac ggaaaagccc gaagaggaac ttgtcttttc |
| 4741 | ccacggcgac ctgggagaca gcaacatctt tgtgaaagat ggcaaagtaa gtggctttat |
| 4801 | tgatcttggg agaagcggca gggcggacaa gtggtatgac attgccttct gcgtccggtc |
| 4861 | gatcagggag gatatcgggg aagaacagta tgtcgagcta ttttttgact tactggggat |
| 4921 | caagcctgat tgggagaaaa taaaatatta tattttactg gatgaattgt tttagtacct |
| 4981 | agatttagat gtctaaaaag cttggcgtaa tcatggtcat agctgtttcc tgtgtgaaat |
| 5041 | tgttatccgc tcacaattcc acacaacata cgagccggaa gcataaagtg taaagcctgg |
| 5101 | ggtgcctaat gagtgagcta actcacatta attgcgttgc gctcactgcc cgctttccag |
| 5161 | tcgggaaacc tgtcgtgcca gctgcattaa tgaatcggcc aacgcgcggg gagaggcggt |
| 5221 | ttgcgtattg ggcgctcttc cgcttcctcg ctcactgact cgctgcgctc ggtcgttcgg |
| 5281 | ctgcggcgag cggtatcagc tcactcaaag gcggtaatac ggttatccac agaatcaggg |
| 5341 | gataacgcag gaaagaacat gtgagcaaaa ggccagcaaa aggccaggaa ccgtaaaaag |
| 5401 | gccgcgttgc tggcgttttt ccataggctc cgcccccctg acgagcatca caaaaatcga |
| 5461 | cgctcaagtc agaggtggcg aaacccgaca ggactataaa gataccaggc gtttccccct |
| 5521 | ggaagctccc tcgtgcgctc tcctgttccg accctgccgc ttaccggata cctgtccgcc |
| 5581 | tttctccctt cgggaagcgt ggcgctttct catagctcac gctgtaggta tctcagttcg |
| 5641 | gtgtaggtcg ttcgctccaa gctgggctgt gtgcacgaac cccccgttca gcccgaccgc |
| 5701 | tgcgccttat ccggtaacta tcgtcttgag tccaacccgg taagacacga cttatcgcca |
| 5761 | ctggcagcag ccactggtaa caggattagc agagcgaggt atgtaggcgg tgctacagag |
| 5821 | ttcttgaagt ggtggcctaa ctacggctac actagaagga cagtatttgg tatctgcgct |
| 5881 | ctgctgaagc cagttacctt cggaaaaaga gttggtagct cttgatccgg caaacaaacc |
| 5941 | accgctggta gcggtggttt ttttgtttgc aagcagcaga ttacgcgcag aaaaaaagga |
| 6001 | tctcaagaag atcctttgat cttttctacg gggtctgacg ctcagtggaa cgaaaactca |
| 6061 | cgttaaggga ttttggtcat gagattatca aaaaggatct tcacctagat ccttttaaat |
| 6121 | taaaaatgaa gttttaaatc aatctaaagt atatatgagt aaacttggtc tgacagttac |
| 6181 | caatgcttaa tcagtgaggc acctatctca gcgatctgtc tatttcgttc atccatagtt |
| 6241 | gcctgactcc ccgtcgtgta gataactacg atacgggagg gcttaccatc tggccccagt |
| 6301 | gctgcaatga taccgcgaga cccacgctca ccggctccag atttatcagc aataaaccag |
| 6361 | ccagccggaa gggccgagcg cagaagtggt cctgcaactt tatccgcctc catccagtct |
| 6421 | attaattgtt gccgggaagc tagagtaagt agttcgccag ttaatagttt gcgcaacgtt |
| 6481 | gttgccattg ctacaggcat cgtggtgtca cgctcgtcgt ttggtatggc ttcattcagc |
| 6541 | tccggttccc aacgatcaag gcgagttaca tgatccccca tgttgtgcaa aaaagcggtt |
| 6601 | agctccttcg gtcctccgat cgttgtcaga agtaagttgg ccgcagtgtt atcactcatg |
| 6661 | gttatggcag cactgcataa ttctcttact gtcatgccat ccgtaagatg cttttctgtg |
| 6721 | actggtgagt actcaaccaa gtcattctga gaatagtgta tgcggcgacc gagttgctct |
| 6781 | tgcccggcgt caatacggga taataccgcg ccacatagca gaactttaaa agtgctcatc |
| 6841 | attggaaaac gttcttcggg gcgaaaactc tcaaggatct taccgctgtt gagatccagt |
| 6901 | tcgatgtaac ccactcgtgc acccaactga tcttcagcat cttttacttt caccagcgtt |
| 6961 | tctgggtgag caaaaacagg aaggcaaaat gccgcaaaaa agggaataag ggcgacacgg |
| 7021 | aaatgttgaa tactcatact cttccttttt caatattatt gaagcattta tcagggttat |
| 7081 | tgtctcatga gcggatacat atttgaatgt atttagaaaa ataaacaaat aggggttccg |
| 7141 | cgcacatttc cccgaaaagt gccacctgac gtctaagaaa ccattattat catgacatta |
| 7201 | acctataaaa ataggcgtat cacgaggccc tttcgtctcg cgcgtttcgg tgatgacggt |
| 7261 | gaaaacctct gacacatgca gctcccggag acggtcacag cttgtctgta agcggatgcc |
| 7321 | gggagcagac aagcccgtca gggcgcgtca gcgggtgttg gcgggtgtcg gggctggctt |
| 7381 | aactatgcgg catcagagca gattgtactg agagtgcacc atatgcggtg tgaaataccg |
| 7441 | cacagatgcg taaggagaaa ataccgcatc aggcgccatt cgccattcag gctgcgcaac |
| 7501 | tgttgggaag ggcgatcggt gcgggcctct tcgctattac gccagctggc gaaaggggga |
| 7561 | tgtgctgcaa ggcgattaag ttgggtaacg ccagggtttt cccagtcacg acgttgtaaa |
| 7621 | acgacggcca gtg |

The vectors of the present invention will be particularly useful for expression of genes in one or more of the hosts listed above and may be used in combination with any functional unit and/or heterologous sequence.

### Methods for Gene Expression

Applicants' invention provides methods for gene expression in host cells, particularly in the cells of microbial hosts, and more particularly, in thermophilic microorganisms. Expression in recombinant microbial hosts, and in particular, thermophilic microorganisms, can be used for the expression of various pathway intermediates, for the modulation of pathways already existing in the host, or for the synthesis of new products heretofore not possible using the host. Additionally, the gene products may be useful for conferring higher growth yields of the host or for enabling the use of alternative growth modes.

Once suitable plasmids are constructed, they are used to transform appropriate host cells. Introduction of the plasmid into the host cell may be accomplished by known procedures such as by transformation, e.g., using calcium-permeabilized cells, electroporation, transduction, or by transfection using a recombinant phage virus (see, e.g., Maniatis, supra).

In one embodiment, the present vectors may be co-transformed with additional vectors, also containing DNA heterologous to the host. It will be appreciated that both the present vector and the additional vector(s) will have to reside in the same incompatibility group. Generally, plasmids that do not compete for the same metabolic elements will be compatible in the same host. Vectors of the present invention comprise the rep protein coding sequence as set forth in SEQ ID NO:21 or variants or fragments thereof as described in detail herein. Any vector containing the instant rep coding sequence and the ORI will be expected to replicate in Thermoanaerobacterium. Any plasmid that has the ability to co-exist with the rep-expressing plasmid of the present invention is in the same compatibility group as the instant plasmid and will be useful for the co-expression of heterologous genes in a specified host.

### Use of Transformed Microbial Hosts for Production Platforms

Once a suitable thermophilic host is successfully transformed with the appropriate vector of the present invention it may be cultured in a variety of ways to allow for the commercial production of the desired gene product. For example, large scale production of a specific gene product, overexpressed from a recombinant thermophilic host may be produced by both batch or continuous culture methodologies.

A classical batch culturing method is a closed system where the composition of the media is set at the beginning of the culture and not subject to artificial alterations during the culturing process. Thus, at the beginning of the culturing process the media is inoculated with the desired organism or organisms and growth or metabolic activity is permitted to occur adding nothing to the system. Typically, however, a “batch” culture is closed with respect to the addition of carbon source and attempts are often made at controlling factors such as pH and oxygen concentration. In batch systems the metabolite and biomass compositions of the system change constantly up to the time the culture is terminated. Within batch cultures, cells moderate through a static lag phase to a high growth log phase and finally to a stationary phase where growth rate is diminished or halted. If untreated, cells in the stationary phase will eventually die. Cells in log phase are often responsible for the bulk of production of end product or intermediate in some systems. Stationary or post-exponential phase production can be obtained in other systems.

A variation on the standard batch system is the “Fed-Batch” system. Fed-Batch culture processes are also suitable in the present invention and comprise a typical batch system with the exception that the substrate is added in increments as the culture progresses. Fed-Batch systems are useful when catabolite repression is apt to inhibit the metabolism of the cells and where it is desirable to have limited amounts of substrate in the media. Measurement of the actual substrate concentration in Fed-Batch systems is difficult and is therefore estimated on the basis of the changes of measurable factors such as pH, dissolved oxygen and the partial pressure of waste gases such as CO₂. Batch and Fed-Batch culturing methods are common and well known in the art and examples may be found in Thomas D. Brock in Biotechnology: A Textbook of Industrial Microbiology, Second Edition (1989) Sinauer Associates, Inc., Sunderland, Mass., or Deshpande, Mukund V., Appl. Biochem. Biotechnol., 36, 227, (1992).

Commercial production of the instant proteins may also be accomplished with a continuous culture. Continuous cultures are an open system where a defined culture media is added continuously to a bioreactor and an equal amount of conditioned media is removed simultaneously for processing. Continuous cultures generally maintain the cells at a constant high liquid phase density where cells are primarily in log phase growth. Alternatively continuous culture may be practiced with immobilized cells where carbon and nutrients are continuously added, and valuable products, by-products or waste products are continuously removed from the cell mass. Cell immobilization may be performed using a wide range of solid supports composed of natural and/or synthetic materials.

Continuous or semi-continuous culture allows for the modulation of one factor or any number of factors that affect cell growth or end product concentration. For example, one method will maintain a limiting nutrient such as the carbon source or nitrogen level at a fixed rate and allow all other parameters to moderate. In other systems a number of factors affecting growth can be altered continuously while the cell concentration, measured by media turbidity, is kept constant. Continuous systems strive to maintain steady state growth conditions and thus the cell loss due to media being drawn off must be balanced against the cell growth rate in the culture. Methods of modulating nutrients and growth factors for continuous culture processes as well as techniques for maximizing the rate of product formation are well known in the art of industrial microbiology and a variety of methods are detailed by Brock, supra.

Consolidated bioprocessing (CBP) is a processing strategy for cellulosic biomass that involves consolidating into a single process step four biologically-mediated events: enzyme production, hydrolysis, hexose fermentation, and pentose fermentation. Implementing this strategy requires development of microorganisms that both utilize cellulose, hemicellulosics, and other biomass components while also producing a product of interest at sufficiently high yield and concentrations. The feasibility of CBP is supported by kinetic and bioenergetic analysis. See van Walsum and Lynd (1998) Biotech. Bioeng. 58:316.

One approach to organism development for CBP begins with organisms that naturally utilize cellulose, hemicellulose and/or other biomass components, which are then genetically engineered to enhance product yield and tolerance. For example, Clostridium thermocellum is a thermophilic bacterium that has among the highest rates of cellulose utilization reported. Other organisms of interest are xylose-utilizing thermophiles such as Thermoanaerobacterium saccharolyticum and Thermoanaerobacterium thermosaccharolyticum. Organic acid production may be responsible for the low concentrations of produced ethanol generally associated with these organisms. Thus, one objective is to eliminate production of acetic and lactic acid in these organisms via metabolic engineering. Substantial efforts have been devoted to developing gene transfer systems for the above-described target organisms and multiple C. thermocellum isolates from nature have been characterized. See McLaughlin et al. (2002) Environ. Sci. Technol. 36:2122. Metabolic engineering of thermophilic, saccharolytic bacteria is an active area of interest, and knockout of lactate dehydrogenase in T. saccharolyticum has recently been reported. See Desai et al. (2004) Appl. Microbiol. Biotechnol. 65:600. Knockout of acetate kinase and phosphotransacetylase in this organism is also possible. Therefore, in certain embodiments, the plasmids and vectors of the present invention may be used to develop organisms for CBP.

An alternative approach to organism development for CBP involves conferring the ability to grow on lignocellulosic materials to microorganisms that naturally have high product yield and tolerance via expression of a heterologous cellulasic system and perhaps other features. For example, Saccharomyces cerevisiae has been engineered to express over two dozen different saccharolytic enzymes. See Lynd et al. (2002) Microbiol. Mol. Biol. Rev. 66:506. Therefore, in certain embodiments, the plasmids and vectors of the present invention may be used to confer the ability to grown on lignocellulosic materials.

Whereas cellulosic hydrolysis has been approached in the literature primarily in the context of an enzymatically-oriented intellectual paradigm, the CBP processing strategy requires that cellulosic hydrolysis be viewed in terms of a microbial paradigm. This microbial paradigm naturally leads to an emphasis on different fundamental issues, organisms, cellulasic systems, and applied milestones compared to those of the enzymatic paradigm. In this context, C. thermocellum has been a model organism because of its high growth rate on cellulose together with its potential utility for CBP.

In certain embodiments, organisms comprising plasmids and vectors of the present invention may be applicable to the process known as simultaneous saccharification and fermentation (SSF), which is intended to include the use of said microorganisms and/or one or more recombinant hosts (or extracts thereof, including purified or unpurified extracts) for the contemporaneous degradation or depolymerization of a complex sugar (i.e., cellulosic biomass) and bioconversion of that sugar residue into ethanol by fermentation.

The following examples illustrate various aspects of the invention, but in no way are intended to limit the scope thereof.

### Examples

### Example 1

Isolation and Sequencing of pMU120

A thermostable plasmid, pMU120 (also referred to herein as pB6A), was isolated from Thermoanaerobacterium saccharolyticum strain B6A, obtained from DSMZ, Braunschweig, Germany under number DSM7060 (also publicly available as ATCC Deposit No. 49915 from the American Type Culture Collection, 10801 University Blvd., Manassas, Va. 20110), using a modified commercial plasmid mini-prep kit (Qiagen™), as follows:

10 ml of an overnight culture of T. saccharolyticum strain B6A was spun down and resuspended in 700 μl of ice cold TE (10 mM Tris pH 8.0, 1 mM EDTA). 500 μl of ice cold acetone was added and the mixture was incubated on ice for 5 minutes. The mixture was microfuged for 1 minute to form a pellet. The supernatant was removed and the pellet was washed by resuspending in 500 μl of ice cold TE. The pellet was microfuged for 1 minute and the supernatant was removed. The pellet was suspended in 250 μl of P1 Buffer (Qiagen™) and 20 μl of lysozyme (50 mg/ml stock in Qiagen™ buffer EB) was added. The mixture was incubated for 20 minutes at 37° C. The next steps of the Qiagen™ plasmid prep protocol were followed according to the manufacturer's directions (Buffer P2-P3, etc.) The optional PB step in the Qiagen™ protocol was also used. 5 μl of the mini-prep was loaded onto a 1% agarose gel containing ethidium bromide. A supercoiled DNA ladder (Invitrogen™) was run alongside of the sample.

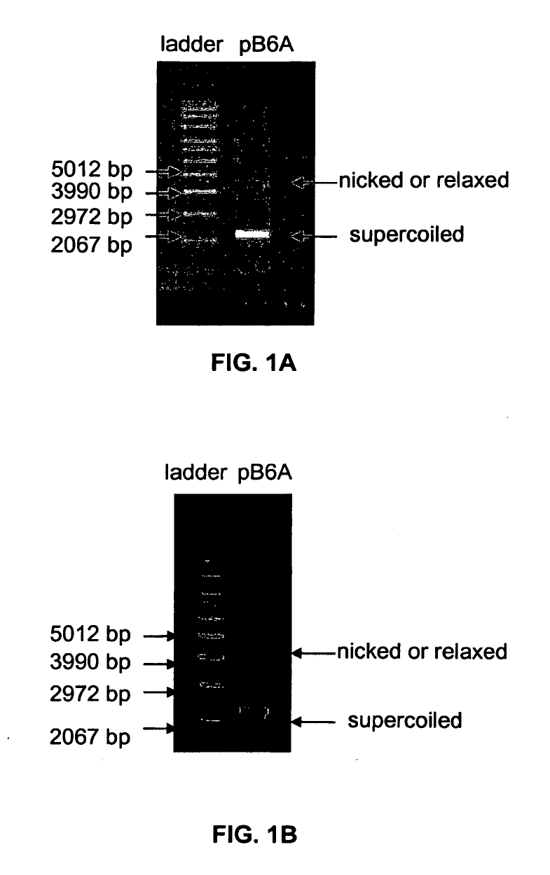

FIG. 1A shows the image of the gel. In the lane labeled “pB6A” there is a predominant band running at approximately 2,300 base pairs, based on the supercoiled DNA ladder, which is the reported size of the native plasmid in strain B6A. See Weimer et al., Arch Microbiol (1984) 138:31-36. There is also a fainter band running at approximately 4,500 base pairs, which is probably a nicked or relaxed form of the plasmid. The smear in the background is most likely genomic DNA contamination.

To further purify pMU120 (pB6A), gel extraction with a commercial gel purification kit (Qiagen™) was used to excise the 2,300 base-pair band. 5 μl of the gel-purified fragment was loaded on a 1% agarose gel containing ethidium bromide. A supercoiled DNA ladder (Invitrogen™) was run alongside of the sample. FIG. 1B shows the image of the gel. After gel purification, the smear of genomic DNA was minimized (FIG. 1B). The larger band at 4,500 base pairs is present after gel purifying the smaller 2,300 base pair band. This suggests that some of the supercoiled plasmid that was gel purified from the 2,300 base pair band changed forms to the relaxed state or was nicked and ran at a larger size.

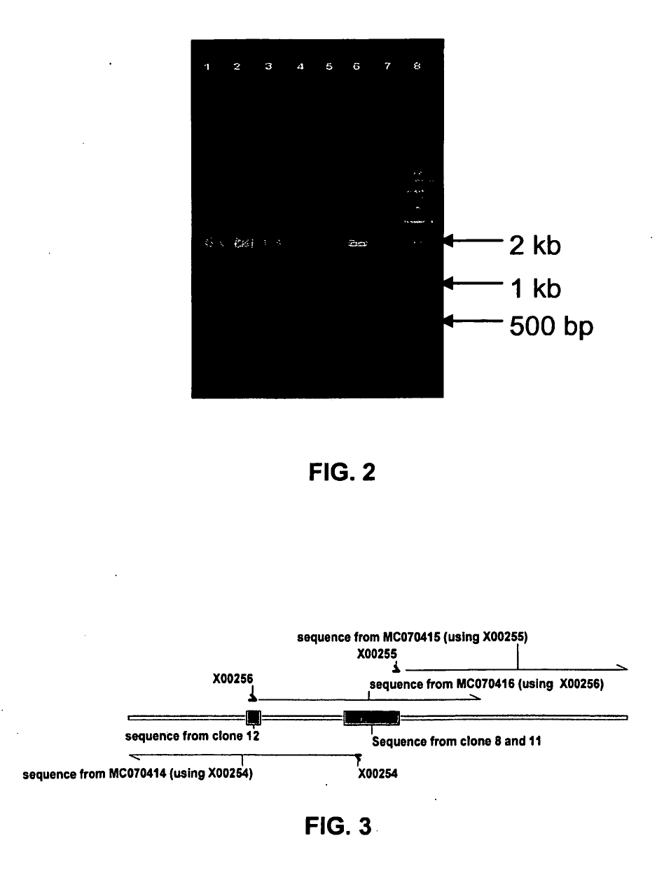

A restriction digest was performed on pMU120 (pB6A) using the restriction enzyme, AseI (FIG. 2). There are multiple AseI cut sites within pMU120 and the digest generated multiple fragments that were less than 500 base pairs and two fragments between 500 base pairs and 1 kilobase (FIG. 2). The AseI digestion products from pMU120 are shown in lane 7 of the gel in FIG. 2.

The restriction enzymes, AseI and NdeI, generate compatible overhangs after digestion. The standard cloning vector, pUC19, has a unique NdeI site. The pUC19 vector was digested with NdeI and the fragments generated from the pMU120 digestion with AseI were cloned into this site. Putative clones containing fragments of pMU120 were screened by digestion with XmnI and EcoRI. These restriction sites are positioned on either side of the NdeI site of pUC19. Thus, clones that have DNA inserted into the pUC19 NdeI site will produce larger DNA fragments after digestion with XmnI and EcoRI. Lanes 1-5 of the gel in FIG. 2 show the results of the XmnI and EcoRI digest performed on the putative clones. Lane 6 of FIG. 2 shows the same digest performed on pUC 19. The clones represented in lanes 1 and 4 of FIG. 2 have inserts that are clearly larger than those found in the control digest (lane 6).

Clones represented in lanes 2, 3, and 5 of FIG. 2 have inserts that are slightly larger than those found in the control digest (lane 6). To determine if inserts were indeed present, the M13 forward primer was used to sequence across the junction region of the NdeI site. The three clones sequenced represent lanes 1, 4, and 5 in FIG. 2. All three clones had DNA inserted in the NdeI site. The clone represented in lane 5 had a 60 base pair insertion and both clones represented in lanes 1 and 4 had identical 235 base pair insertions.

The DNA sequence of the 60 base pair insertion is:

(SEQ ID NO: 1)

5′GATTATAAAGAGAAGGGACTTTATTTATTAAGTAGTATGGATGATTT
TATTAAAATTATG 3′

The DNA sequence of the 235 base pair insertion is:

(SEQ ID NO: 2)

5′ATTGTTAGCACTGGGCCTGGTCTGCGGCCCAGACCCGAAATTATGCA
AAATAAAATGATATTTTATTATGATTATGAACATCTCTAAAAGCCTTAA
AAATTGTAAAAGAATTAAAAGAAAAATAAAAACCACACGTTTTGTTAA
ACTCATCAAAAAAAACTAATTCAGAAACTTTGATAGAAGGGAATAAAT
CAACACTAAAATCTAAACTTAAAGATTTCCTATTAAATTTTCT 3′

The above DNA sequences were used to design, by visual inspection, three primers that were used to obtain additional sequence from the plasmid. The primer sequences are as follows (5′-3′):

(SEQ ID NO: 3)

Primer X00254: CAGAAACTTTGATAGAAGG.

(SEQ ID NO: 4)

Primer X00255: CAGACCAGGCCCAGTGCTAAC.

(SEQ ID NO: 5)

Primer X00256: GGACTTTATTTATTAAGTAGTATGG.

The above primers were used in sequencing reactions with pMU120 (pB6A) as the template. Vector NTI was used to assemble all of the DNA fragments (fragments that were cloned into pUC 19 and those obtained by DNA sequencing). The assembled sequence was 2,085 base pairs. A map of the assembly and the locations of each fragment are shown in FIG. 3. The sequence of the assembly is represented by SEQ ID NO:6, below:

(SEQ ID NO: 6)
TAAAGATTTATATTTTGCATTACATAATGTTCATCAGATACAAGGCTATGGTTGTTTATATAATATAAGAGATGATA
CTCAATTAGATTTAAAGGTTGATGACATTTATAATGATTTGATTGATTTATTACAAGTTACAGAAAATCCTATACAG
TCTATGGAAACTGTACAGGATTTATTAAAGGATACTGAATATACAATAATAAGCCGTAAGCGTATATTTAAGTATC
TAACACAATTATATCATAAGGATTGATATTTATACCGTCTGTCGGACTCATGCGGAGGGGGACTTGAGGGGGTCTC
CCCTCGCATTGTACGACAGACGGTATTATTATTATACAAATTTTTTTTATGTAATTTTTTTTGTGTAATTTTTTTATAC
AAATAATATTTCAATTGACAAAGTTTTCTATTTGTGTTAACATTGTTTATATAATAGTGAACAGTGTTAAGATTAAA
TGTGAGGTGTTTGTATGGATATTAATGATTATAAAGAGAAGGGACTTTATTTATTAAGTAGTATGGATGATTTTATT
AAAATTAATGATTTGTTTATGGGTAAAGTTGTTTCTCCTGGCTATGTTGCTTCGGTTTTTGGTGTTTCCAGGTCTACT
GTTACACAATGGATTCAAAGACGTAAAATTAGAGCTTTTAAGTATAAAGGTAAGGAAGGTGACTATATGGTTATAC
CTATTGCTGATATTATTGATTACAAAAGATTGAGTAATAATGATTTTATTTATGATAAGTTAGTGAGGTGATTTATT
TTATGTTTGACGATAGCTATGTTGTTAATGAGTGTTCGTCTAATGTTAGTGAAAATGATAGAGATTTTTGTAGTTTG
GTTGGTCGTTTTATGATTATTAATGGTATAGATAAGTTGGTTATTAAGATTAATAGAAAATTTAATAGGAAATCTTT
AAGTTTAGATTTTAGTGTTGATTTATTCCCTTCTATCAAAGTTTCTGAATTAGTTTTTTTTGATGAGTTTAACAAAAC
GTGTGGTTTTTATTTTTCTTTTAATTCTTTTACAATTTTTAAGGCTTTTAGAGATGTTCATAATCATAATAAAATATC
ATTTTATTTTGCATAATTTCGGGTCTGGGCCGCAGACCAGGCCCAGTGCTAACAATATTAATTTTTAATGTTAGGAA
TTGTTTAATTCTTAATTGTGTTTTTAAAGGTAGAATAATTACCCATTCGCCCTTTAGCCAACAAAAATTAAGGAGGT
ATAAACATGGATAAAATGGATTTGATTCTTCAAGATGAAAGACTGGGTGAGATATTTAAAGATATAGATTTAACAG
ATAATGAAAAGAGATATCTTAAATGGTTATGGAAATGGGATTATGAAACACGTGATACTTTTGTATCAATTTTTTTT
GAAGCTAAAAAATGGTGGAAAATGATTTTTTTTCTTATCTTGATATATTAGAAAAAAGCGTACTCACGAAGTAAGA
ATTTGTAAAAAAAGAAGGGGGGATTTTTTTGGATGAGAGTTTGTACAAGCAGATTTTAAGTAATATTATTATTACTC
GTGATTATTGTAAAAATGTTTTAGATAATATAAAGTTCAATGAAAAAATAATTGATTATTATGTTATGTTACAAAAT
GATGTTTTTATTGATTTTACTAATAAAATAAATTCAATAAGGGATTGTAATAAATATTGGTATTTGGATGTTTATAA
AAAGCAGAAAATAAAGGATTTTAAAAAGACTAATTTGTGTAAAGATAAGTTCTGTAATAATTGTAAGAAAGTTAA
ACAGGCTTCAAGAATGCAAAAATATATTCCTGAATTACAGAAATACAAAGATGGCTTATATCATTTTATATTTACT
GTTGAAAATGTGCCAGGTAGTGAATTAAGAGATACTATTGATAGGTTGTTTAAGTCTTTTAAGTCATTTACAAGGTA
TTTAAGTGGTAATCTTAAAATAAAAGGTGTTAATTTTGATAAATGGGGTTATAAAGGCTGTGTAAGGTCTTTAGAG
GTAACTTATAGTATGATTGATAATCATATTATGTATCATCCACACTTGCATGTTGCGATGATATTAGATCCTTTTTAC
GATGGGTTA

Because the plasmid was predicted to be approximately 2.3 kb and the sequence assembly generated did not overlap at the ends, additional sequence information was needed. So the assembly sequence of SEQ ID NO:6 was used to design additional primers for further DNA sequencing. These primers were as follows (5′-3′):

(SEQ ID NO: 7)

Primer X00316: CCTGTACAGTTTCCATAGAC.

(SEQ ID NO: 8)

Primer X00317: GGTTATAAAGGCTGTGTAAGG.

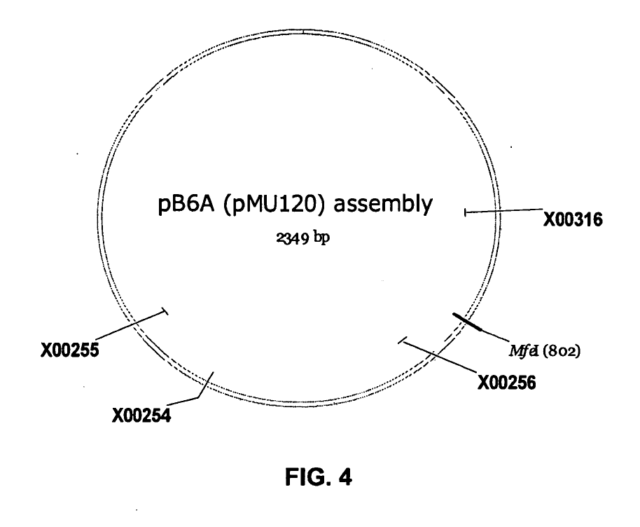

The above primers were used in sequencing reactions with pMU120 (pB6A) as the template. The reaction with the primer represented by SEQ ID NO:8 was unsuccessful. However, the sequencing reaction with the primer represented by SEQ ID NO:7 generated enough sequence to fill the gap, allowing a complete sequence map of pMU120 (pB6A) to be generated in Vector NTI (Invitrogen™). The sequencing reactions were repeated for confirmation. The second round of sequencing differed from the first round at only two bases, both of which were near the ends of sequencing reactions, in the middle of large stretches of Ts. Based on the two rounds of sequencing, a vector map was generated in Vector NTI (Invitrogen™). This map (including the locations of the primers) is shown in FIG. 4.

The entire sequence of pMU120 (pB6A) is 2,349 base pairs and is represented by SEQ ID NO:9.

### Analysis of Open Reading Frames

The sequence of pMU120 (SEQ ID NO:9) was analyzed using the open reading frame (orf)-finding properties built into Vector NTI (Invitrogen™). When a cut-off of 50 codons was assigned as the minimum orf size, six orfs were recognized. These are shown as arrows in the vector map of FIG. 5.

Each orf was searched (“blasted”) using the blastx algorithm on the NCBI website (ncbi.nlm.nih.gov/BLAST). Only the largest orf had significant homology to any sequences in the existing database. The translated protein encoded by this orf was most homologous to the RepB protein (Accession No. CAA44562), which is encoded on a cryptic plasmid (pCB101) found in Clostridium butyricum. This protein is involved in DNA replication. Replication proteins typically bind to the plasmid DNA and nick it at the single- or double-strand origin of replication.

In addition to the blastx algorithm, the entire nucleotide sequence of the plasmid was referenced against a nucleotide database using the blastn algorithm on the NCBI website (ncbi.nlm.nih.gov/BLAST). As expected, a portion of the repB gene of pCB101 was homologous to the repB oil of pMU120. Furthermore, two small regions (one of 40 base pairs and another of 48 base pairs) of an indigenous plasmid found in Clostridium MCF-1 were 87% and 90% identical at the nucleotide level, respectively, to portions of the pMU120 repB orf.

### Example 2

### Engineering a Shuttle Vector

The sequence information obtained in Example 1, above, was used to engineer a shuttle plasmid with the ability to replicate both in thermophilic organisms and in E. coli hosts. First, plasmid from strain B6A (pMU120) was ligated into pUC19. Plasmid pMU120 has a unique MfeI site (see plasmid map in FIG. 5). DNA digested with MfeI has the same overhangs as DNA digested with EcoRI. Thus, pMU120 that has been digested with MfeI can be cloned into the unique EcoRI site found on pUC19.

Plasmid pMU120 was cut with MfeI and pUC19 was cut with EcoRI. Plasmid pMU120 was ligated into pUC19, then electroporated into TOP10 competent cells (Invitrogen™) and selected on ampicillin. Plasmid DNA was prepared from 4 colonies. Restriction digests of the eluted plasmids were set up using NdeI plus HindIII. One mini-prep had two bands, one of about 2.6 kb and one of about 2.4 kb, while pUC had only one band of about 2.6 kb. This was as expected, as shown in the plasmid in FIG. 6 (note that the EcoRI site in pUC19 has been destroyed).

This new plasmid, designated pMU121 (pB6ApUC), is 5035 base pairs and is represented by SEQ ID NO:10.

### Addition of a Kanamycin Marker

The construct pIKM1 was digested with HindIII, which liberates three fragments, the smallest of which (˜1.4 kb) contains the kanamycin resistance gene with a suspected promoter. This fragment was gel purified. The construct pMU121 was also digested with HindIII. These DNAs were ligated then transformed into TOP10 E. coli cells (Invitrogen™) and plated on kanamycin. Plasmid DNA was prepared from six colonies. To test that they ligated correctly, the plasmid DNAs were digested with PciI plus BamHI. Digestion of all the potential clones resulted in two bands of approximately 4,646 base pairs and approximately 1,757 base pairs, as expected (see map in FIG. 7). This construct has been named pMU131.

The sequence of pMU131, which is 6,403 base pairs, is represented by SEQ ID NO:11.

### Example 3

Transformation of pMU131 into T. saccharolyticum

DNA of pMU131 was transformed into wild-type T. saccharolyticum strain YS485 using a method based on those described previously (Mai, V., W. W. Lorenz, and J. Wiegel. 1997. “Transformation of Thermoanaerobacterium sp. strain JW/SL-Y485 with plasmid pIKM1 conferring kanamycin resistance.” FEMS Microbial. Lett. 148:163-167 and Tyurin M. V., Desai S. G., Lynd L. R. 2004. “Electrotransformation of Clostridium thermocellum.” Appl Environ Microbiol. 70:883-890) and selection was performed for kanamycin resistance. Transformations were performed with the resulting number of cfu/ml/μg DNA shown in Table 1, below:

TABLE 1

| Transformation | pMU131 | pMU130 | pHK03 |
|---|---|---|---|
| 1 | 600 | 0 | — |
| 2 | 12000 | 0 | 3600 |
| 3 | 19080 | 24 | >12000 |

pMU130 is a plasmid derived from pIKM1, a published T. saccharolyticum plasmid (Mai, V., W. W. Lorenz, and J. Wiegel. 1997. “Transformation of Thermoanaerobacterium sp. strain JW/SL-Y485 with plasmid pIKM1 conferring kanamycin resistance.” FEMS Microbial. Lett. 148:163-167).

pHK03 is a non-replicating suicide plasmid obtained from Arthur J. Shaw, designed to replace a T. saccharolyticum gene encoding hydrogenase-1 with a kanamycin resistance gene. It was derived from the cloning vector pBluescript II SK(+) by adding sequences flanking the hydrogenase-1 gene and the kanamycin resistance gene.

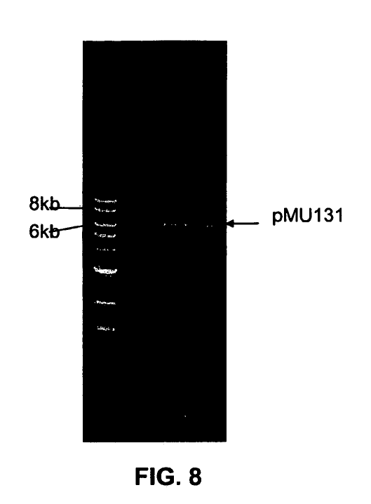

These results show that pMU131 readily transforms T. saccharolyticum at a much higher efficiency than a plasmid derived from pIKM1. These results also suggest that a replicating plasmid transforms more efficiently than a suicide plasmid. Transformation was confirmed by recovering plasmid DNA from the T. saccharolyticum strains and digesting with BamHI (upon BamHI digestion a 6.4 kb band is expected). As shown in FIG. 8, this is the case. Two candidates produced a plasmid of approximately 6.4 kb, the size expected for pMU131 (FIG. 8). The marker used was the NEB 1 kb ladder.

### Example 4

Adding Chloramphenicol and Erythromycin Markers to pMU121

The chloramphenicol and erythromycin resistance genes from pJIR418 were amplified using the following primers (5′-3′):

(SEQ ID NO: 12)

Primer X00385: ggcgAAGCTTggtctttgtactaacctgtgg

(SEQ ID NO: 13)

Primer X00388: GGCGaagcttGAG TTA GCT CAC TCA TTA
GG

These primers were engineered with HindIII sites, so the resulting PCR product, along with pMU121, was digested with HindIII. After CIP-treatment, the pMU121 and PCR product were ligated together. This resulted in a construct, pB6ApUCcatery (pMU141) as shown in FIG. 9

The sequence of pMU141, which is 7106 base pairs, is represented by SEQ ID NO:14.

The chloramphenicol resistance gene from pJIR418 was amplified using primers (5′-3′):

(SEQ ID NO: 15)

Primer X00385: ggcgAAGCTTggtctttgtactaacctgtgg.

(SEQ ID NO: 16)

Primer X00386: GGCGaagcttCTA CTG ACA GCT TCC AAG
GAG.

These primers were engineered with HindIII sites so the resulting PCR product, along with pMU121, was digested with HindIII. After CIP-treatment, the pMU121 and PCR product were ligated together. This resulted in a construct, pB6ApUCcat (pMU144), as shown in FIG. 10.

The sequence of pMU144, which is 6,045 base pairs, is represented by SEQ ID NO:17.

The erythromycin resistance gene from pJIR418 was amplified using the following primers (5′-3′):

(SEQ ID NO: 18)

Primer X00387: ggcgAAGCTTctccttggaagctgtcagtag.

(SEQ ID NO: 19)

Primer X00388: GGCGaagcttGAG TTA GCT CAC TCA TTA
GG.

These primers were engineered with HindIII sites so the resulting PCR product, along with pMU121, was digested with HindIII. After CIP-treatment, the pMU121 and PCR product were ligated together. This resulted in a construct, pB6ApUCery (pMU143), as shown in FIG. 11.

The sequence of pMU143, which is 6,143 base pairs, is represented by SEQ ID NO:20.

### Example 5

Determination of the pMU120 Origin of Replication (ORI)

The origin of replication of pMU120 (pB6A) was determined by aligning the origin of replication sequences of gram-positive rolling circle plasmids pAO1, pC194, pNB2, pUB110, pBC1, pBAA1, pBAS2, and pLS11 to derive the following consensus on sequence: TTTTTTCTTATCTTGATA TATAT (SEQ ID NO:29). See, e.g., Clausen et al., Plasmid (2004) 52:131-8. A map of the pMU120 plasmid, including the origin of replication, is shown in FIG. 5.

Vector NTI was used to search the pMU120 (pB6A) DNA sequence for the TCTTAT sequence found within SEQ ID NO:29, which was completely conserved among the different ORIs. The sequence was located in a single location spanning base pairs 1822-1827 of pMU120 (amino acids 1822-1827 of SEQ ID NO:9). The region surrounding the TCTTAT sequence of pMU120 was aligned with the ori sequences of the eight gram-positive rolling circle plasmids listed above listed above using Vector NTI. The result of the alignment is shown below:

| | | |
|---|---|---|
| | 1 25 | |
| pB6A ori | TTTTTTCTTATCTTGATA-TATTA- | (SEQ ID NO: 30) |
| pAO1 ori | TTTTTTCTTATCTTGATCA-AGTGT | (SEQ ID NO: 31) |
| pC194 ori | TTCTTTCTTATCTTGATAATAACG- | (SEQ ID NO: 32) |
| pNB2 ori | TTTTCTCTTATTCTGTTTTAATAC- | (SEQ ID NO: 33) |
| pUB110 ori | TTCTTTCTTATCTTGATA-CATAT- | (SEQ ID NO: 34) |
| pBC1 ori | TTTTTTCTTATCTTGATAATATAT- | (SEQ ID NO: 35) |
| pBAA1 ori | TCTTTTCTTATCTTGATAGTATAT- | (SEQ ID NO: 36) |
| pBAS2 ori | TTTATTCTTATCTATGTA-TATAT- | (SEQ ID NO: 37) |
| pLS11 ori | TTTTTTCTTATCTTGATACTATAT-- | (SEQ ID NO: 38) |
| Consensus | TTTTTTCTTATCTTGATA TATAT | (SEQ ID NO: 29) |

The alignment indicates that pB6A has a conserved gram-positive rolling circle origin of replication.

### Example 6

Addition of a Yeast Marker/Replicon to pMU121 to Generate pMU158

The pMU158 was generated by linearizing the plasmid pMU121 plasmid and adding a yeast selectable marker and a yeast origin of replication. As shown in FIG. 6B, the pMU121 plasmid has a unique SapI site. The plasmid pMU121 was digested overnight with the SapI restriction enzyme in a reaction volume of 20 μl containing 5.0 μl of pMU121, 2 μl buffer 4, 1 μl SapI and 12 μl dH2O. 5 μl of SapI digested pMU121 plasmid was run on a 1% agarose gel. As shown in FIG. 14A, the Sap I restriction digest reaction generated a DNA corresponding to the predicted size (approximately 5 kb) of a linearized pMU121 plasmid.

A yeast Ura3-CEN6/ARSH amplicon was generated by PCR amplification of plasmid pMU110 using primers X00592 and X00593. A map of the pMU110 plasmid is shown in FIG. 11. The sequence of primers 592 and 593 are (5′-3′):

(SEQ ID NO: 23)

Primer X00592: Ctttccagtcgggaaacctgtcgtgccagcagatc
tgatcgcttgcctgtaacttac.

(SEQ ID NO: 24)

Primer X00593: GCC TTT GAG TGA GCT GAT ACC GCT CGC
CGC AGA TCT CGA AAA GTG GGT AAT AAC TG.

The PCR amplification reaction was performed in a total reaction volume of 100 μl having 1.0 μl of pMU110 (template), 1.0 μl of primer X00592 (100 μM), 1.0 μl of primer X00593 (100 μM), 4.0 μl of dNTP's (2.5 mM stock), 10.0 μl of Taq Buffer, 1.0 μl of Taq Polymerase, and 82.0 μl of dH2O. As shown in FIG. 14B, the amplified Ura3-CEN6/ARSH sequence is of the predicted size (approximately 1.7 kB).

The Ura3-CEN6/ARSH amplicon and SapI-linearized pMU121 plasmid were ligated together using a yeast mediated ligation reaction as follows: (1) S. cerevisiae cells were cultured overnight in yeast minimal medium (YPD); (2) 0.5 mL of overnight yeast culture was added to a 1.5 mL microfuge tube and cells were spun down at 8-10K for 10 seconds. The supernatant was removed and washed with 0.5 mL sterile TE. (3) To the cell pellet, 0.5 mL “Lazy Bones Solution,” 20 μL of carrier DNA (Salmon sperm DNA at 2 mg/mL), and plasmid DNA (5 μl of linear DNA) was added. If in vivo cloning were performed the second DNA (entire PCR reaction) would be added at this time as well. The “Lazy Bones Solution” contained 40% Polyethylene glycol (MW 3350; Sigma P3640), 0.1 M Lithium acetate (LiAc), 10 mM Tris-HCl (pH 7.5), 1 mM EDTA. The single-stranded carrier DNA contained high-molecular-weight DNA (Deoxyribonucleic acid Sodium Salt Type III from Salmon Testes; Sigma D1626). The TE buffer (pH 8.0) corresponded to 10 mM Tris-Cl pH 8.0, 1 mM EDTA; (4) The cells with added solution were vortexed hard for 1 minute; (5) Cells were then incubated overnight at room temperature; (6) After overnight incubation, cells were heat shocked for 10-12 minutes at 42° C.; (7) Cells were pelleted, washed with TE, and plated onto selective plates (lacking uracil) and incubated at 30° C.

The DNA from colonies selected above was extracted using the “smash and grab” protocol. The “smash and grab” protocol is a method to release plasmids from S. cerevisiae for transformation into E. coli. based on Hoffman and Winston, Gene 57:267-272 (1987) and was performed as follows: (1) Yeast transformants were scraped off of the agar surface using a spreader and 2 ml of sterile TE buffer. After centrifugation, the final volume of cells was approximately 50-100 μL in a graduated microfuge tube; (2) 0.2 mL of “Smash and Grab” buffer were added and the pellet was resuspended. The “Smash and Grab” Buffer contained 1% SDS, 2% Triton X-100, 100 mM NaCl, 10 mM Tris-HCl pH 8.0, and 1 mM EDTA. Next, 0.3 g of 0.5 mm glass beads were added. Then, 0.2 mL phenol: chloroform: isoamyl alcohol (25:24:1) was further added; (3) The resulting suspension was vortexed at high speed for 2 minutes; (4) The vortexed suspension was then centrifuged for 5 minutes in a microcentrifuge; (5) The aqueous phase was removed by pipetting and transferred to a new 1.5 ml tube. 0.7 volumes isopropanol was added, mixed, and set aside for 5 minutes at room temperature; (6) The solution was then spun down in a microfuge tube for 5 minutes at high speed; (7) The supernatant was removed and the pellet was washed twice with 70% Ethanol (0.5 mL); (8) The pellet was dried briefly and then resuspended in 30 μL TE or water. 3.0 μL of the resuspended pellet was then transformed into E. coli.

Three colonies of potential E. coli transformants were picked and grown overnight in LB ampicillin (100 μg/ml). The following day the DNA from the overnight cultures were miniprepped and digested with either BamHI and Nco I, or BglII alone.

As shown in FIG. 14C, the BamH1/NcoI digestion of the pMU158 plasmid resulted in the predicted 5.4 and 1.2 kb bands in two of the three clones analyzed. As shown in FIG. 14D, the Bgl II digestion of the pMU158 plasmid resulted in the predicted 4.9 and 1.6 kb bands in two of the three clones analyzed.

A map of the resulting plasmid, pMU158, is shown in FIG. 13. The sequence of pMU158, which is 6589 bp, is represented as SEQ ID NO: 25.

### Example 7

Adding a Selectable Marker to pMU158 to Generate pMU166

The pMU158 plasmid was used to generated the pMU166 plasmid, which contains a selectable marker for T. saccharolyticum.

As shown in FIG. 13, the pMU158 plasmid has a unique BsrFI site in the amplicillin (Ap) resistance cassette that can be used to linearize the plasmid and insert a Kn cassette in its place using yeast mediated ligation. The pMU158 plasmid was digested overnight with BsrFI in 20 μl reaction volume containing 5.0 μl of pMU158 plasmid, 2 μl buffer BsrFI, 1 μl BsrFI and 12 μl dH2O.

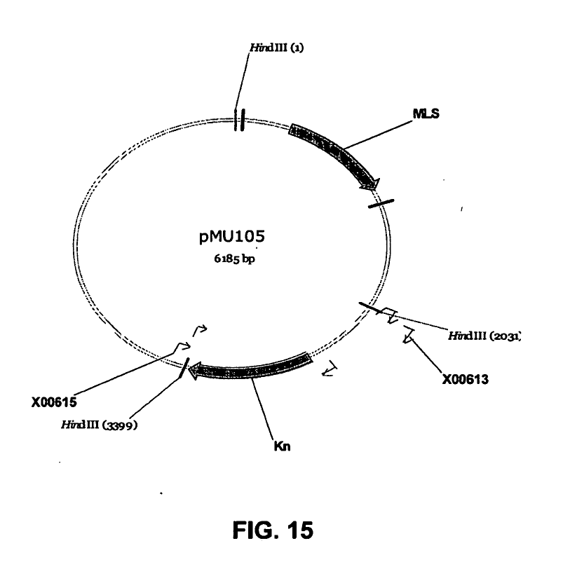

A DNA fragment containing the kanamycin (Kn) resistance selectable marker was generated by PCR amplification of the pMU105 plasmid using primers X00613 and X00615. A map of the pMU105 plasmid is shown in FIG. 15. The X00613 and X00615 primers (5′-3′) are as follows:

(SEQ ID NO: 26)

Primer X00613: AATGTGCGCGGAACCCCTATTTGTTTATTTaaccc
agcgaaccatttgag.

(SEQ ID NO: 27)

Primer X00615: aatgaagttttaaatcaatctaaagtatatAGA
GTC GAT ACA AAT TCC TCG.

PCR amplification was performed in a 100 μl reaction volume containing 1.0 ul of pMU105 diluted 1:100 (template), 1.0 ul of primer 613 (100 uM), 1.0 ul of primer 615 (100 uM), 4.0 ul of DNTP's (2.5 mM stock), 10.0 ul of Taq Buffer, 1.0 ul of Taq Polymerase and 82.0 ul of dH2O.

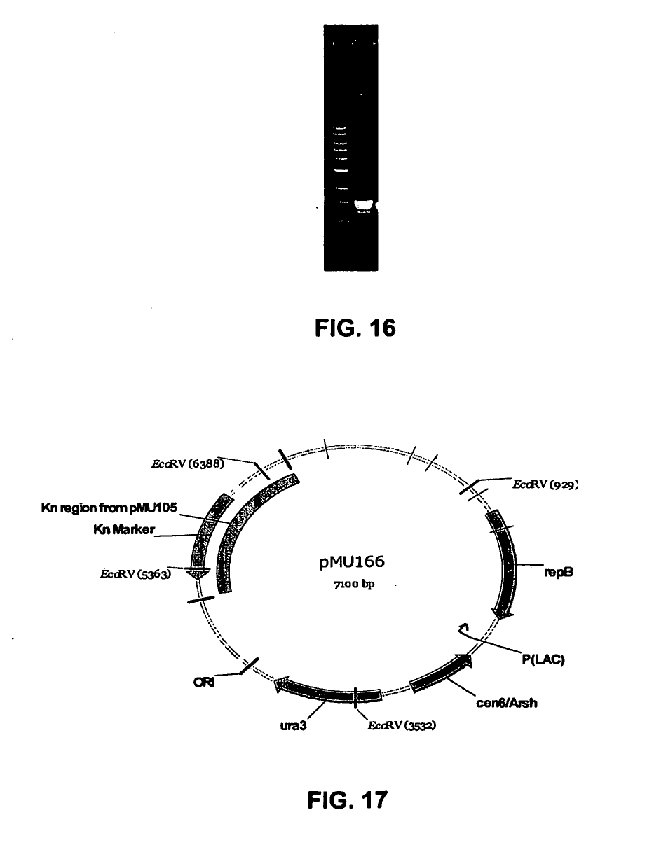

As shown in FIG. 16, the amplified Kn sequence is of the predicted size (approximately 1,475 bp). The Kn amplicon and linearized pMU105 vector were used in a yeast mediated ligation reaction, as described above. Colonies that resulted from the yeast mediated ligation reaction were subjected to the “smash and grab” protocol, as described above, to isolate plasmid from the yeast and transform E. coli, and select on kanamycin for the insertion of the new marker.

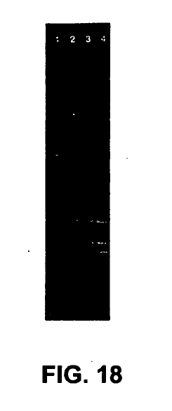

Three Kn-resistant E. coli colonies were selected and DNA was isolated by miniprep and subjected to a diagnostic EcoRV digest. As shown in FIG. 18, Eco RV digestion of the ligated plasmid resulted in the predicted 2.6, 1.8, 1.6, 1.0 kb bands in all three clones. A map of the resultant plasmid pMU166, showing the EcoRV sites is shown in FIG. 17. The sequence of pMU166; which is 7000 bp, is represented as SEQ ID NO: 28.

During the construction of the pMU166 plasmid, as described above, the plasmid was cultured both in S. cerevisiae and E. coli. Thus, the pMU166 plasmid was maintained in both of these hosts. It was also successfully transformed into T. saccharolyticum. The pMU166 plasmid is therefore capable of functioning as an E. coli-S. cerevisiae-thermophile shuttle vector.

### Example 8

pMU675-pyrF (Ura3) expression in T. Saccharolyticum

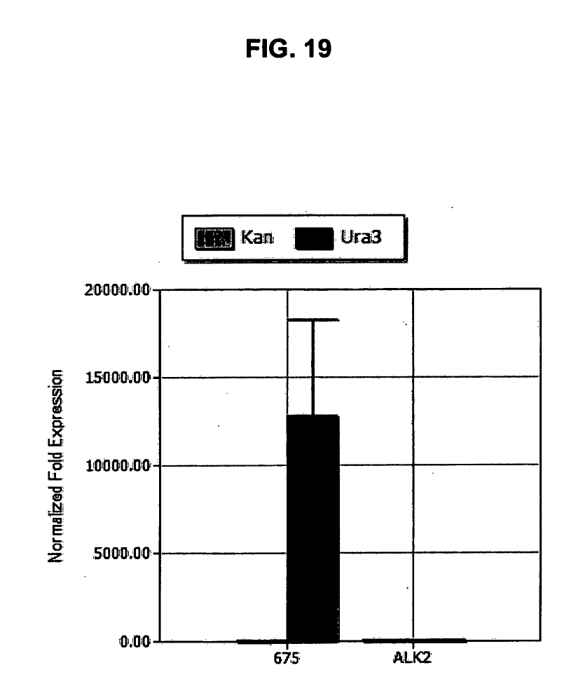

A nutritional marker was used as a selective agent carried on the B6A plasmid. The pyrF (commonly referred to as Ura3) gene, encoding orotidine 5-phosphate decarboxylase activity (EC 4.1.1.23) is required for de novo uracil synthesis. A T. saccharolyticum JW/SL-YS485 strain with a Ura3 deletion requires external supplementation of uracil in order to grow. When the Ura3− strain was transformed with a B6A-derived plasimd containing the native T. saccharolyticum Ura3 gene, the ability to grow without uracil supplementation was restored. Expression of the plasmid carried Ura3 gene was 10,000 fold higher than the native Ura3 expression level (FIG. 19).

### Plasmid Construction and Experimental Results

The pMU675 vector was constructed by independent PCR amplification of the kanamycin selectable marker, the C. thermocellum CBP promoter, the T. saccharolyticum Ura3 gene, and the T1+T2 terminator sequence. The PCR products were fused and inserted into the pMU158 backbone using yeast-mediated ligation and subsequently transformed into E. coli. The vector was confirmed using PCR and restriction enzyme diagnostics. pMU675 was then transformed into Ura3− T. saccharolyticum mutants containing a deletion in the Ura3 gene by first using kanamycin selection followed by selection on defined medium without uracil. The transformants were successful in growing on medium without uracil, indicating that autotrophy was restored to the Ura3− strain by the expression of the native Ura3 gene from the pMU675 plasmid. Ura3 expression was further monitored using real-time PCR. RNA was isolated from the pMU675 transformed T. saccharolyticum cultures using the Qiagen® RNeasy Mini Isolation kit and cDNA prepared using the Invitrogen® Thermoscript cDNA Synthesis Kit. Real-time expression was monitored using Bio-Rad® SYBR Green and normalized to the T. saccharolyticum ribosomal recycling factor housekeeping gene. Expression of the Ura3 gene, under control of the CBP promoter, was greater than 10,000 fold higher in pMU675 harboring T. saccharolyticum when compared to native Ura3 expression in the Ura3+ strain ALK2 (FIG. 19).

### Example 9

pMU362—Thiamphenicol Selection in Tsacc

An additional antibiotic selection gene is shown to function with the B6A plasmid for selection in T. saccharolyticum JW/SL-YS485.

### Plasmid Construction and Experimental Results

The catD chloramphenicol resistance-conferring gene and its native promoter were PCR amplified from the pMU180 vector (carrying the catD gene from the plasmid known to the art as pJIR418, see, e.g., Rood and Cole, Microbiol. Rev. 55: 621-648 (1991)) and cloned into the pCR2.1-TOPO TA cloning vector. The fragment was gel purified from the TOPO vector and ligated into the pMU131 vector using the BamHI and PstI restriction sites. The ligation product was transformed into Top10 chemical competent E. coli and selected on LB-Chloramphenicol 25 μg/ml plates. The plasmid was PCR screened (Figure A) using the cloning primers and further screened with a BamII+EcoRV and SacI+ApalLI digest (Figure B). The resulting plasmid was annotated as pMU362.

The pMU362 vector was successfully transformed into YS485 T. saccharolyticum using 10 μg/ml thiamphenicol on pH 6.1 M122C medium, incubated at 48° C. for approximately 72-96 h. The table below provides one example of a successful transformation at 48° C.

Table A shows a T. saccharolyticum colony count of catD vector transformation after 96 h incubation at 48° C., plated in 100 μl or 1000 μl volumes.

Table B shows the OD of the initial transformation culture and the final OD after the 3 h incubation, just prior to plating

A

| | |
|---|---|
| Kan | Thiam |

| | 100 ul | 1000 ul | 100 ul | 1000 ul |
|---|---|---|---|---|
| pMU131 | 240 | 1254 | 0 | 0 |
| pMU362 | 250 | 1490 | 45 | 648 |

B

| | Initial | Final |
|---|---|---|
| pMU131 | 0.08 | 0.56 |
| pMU362 | 0.08 | 0.48 |

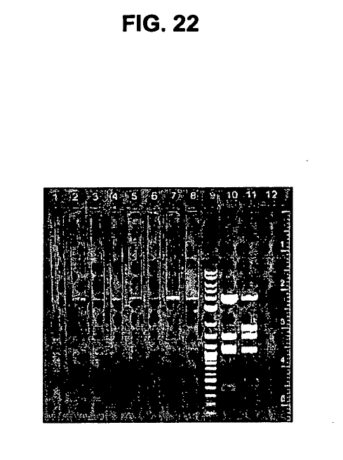

To further confirm successful transformation and selection, a plasmid isolation was performed on 8 random pMU362 transformed T. saccharolyticum colonies using the plasmid isolation protocol described in example 1. Plasmid isolations were screened with an EcoRV+SacI double digest to determine the presence of the pMU362 vector. FIG. 22 provides evidence that the pMU362 transformation was successful and the thiamphenicol resistance is due to the catD gene.

All publications such as textbooks, journal articles, GenBank or other sequence database entries, published applications, and patent applications mentioned in this specification are herein incorporated by reference to the same extent as if each individual publication or patent application was specifically and individually indicated to be incorporated by reference. This application claims the benefit of U.S. Provisional Application No. 60/971,225, filed Sep. 10, 2007, the entire contents of which are incorporated herein by reference.

What is claimed is:

1. An isolated nucleic acid comprising a sequence that is at least about 90% identical to SEQ ID NO:21, wherein said nucleic acid does not consist only of the plasmid pB6A of SEQ ID NO:9 or the plasmid isolated from T. Saccharolyticum type strain B6A deposited as ATCC No. 49915.

2. The isolated nucleic acid of claim 1 comprising a sequence that is at least about 95% identical to SEQ ID NO:21.

3. The isolated nucleic acid of claim 2 comprising a sequence that is at least about 99% identical to SEQ ID NO:21.

4. The isolated nucleic acid of claim 3 comprising the sequence of SEQ ID NO:21.

5. An isolated nucleic acid comprising a sequence that encodes a polypeptide that is at least about 90% identical to the amino acid sequence of SEQ ID NO:22, wherein said nucleic acid does not consist only of the plasmid pB6A of SEQ ID NO:9 or the plasmid isolated from T. Saccharolyticum type strain B6A deposited as ATCC No. 49915.

6. The isolated nucleic acid of claim 5, comprising a sequence that encodes a polypeptide that is at least about 95% identical to the amino acid sequence of SEQ ID NO:22.

7. The isolated nucleic acid of claim 5, comprising a sequence that encodes a polypeptide that is at least about 99% identical to the amino acid sequence of SEQ ID NO:22.

8. A plasmid comprising the isolated nucleic acid of any of claims 1-7, wherein said plasmid does not consist only of the plasmid pB6A of SEQ ID NO:9 or the plasmid isolated from T. Saccharolyticum type strain B6A deposited as ATCC No. 49915.

9. The plasmid of claim 8, wherein said plasmid is replicative and stable in a thermophilic host.

10. The plasmid of claim 8 or 9, wherein said plasmid further comprises at least one functional unit.

11. The plasmid of claim 10, wherein said functional unit is selected from the group consisting of: a replicon, an origin of replication, a sequence encoding a protein or a functional protein fragment, a restriction site, a multiple cloning site, and any combination thereof.

12. The plasmid of any one of claims 8-11, wherein said plasmid comprises a gram-positive rolling circle origin of replication.

13. The plasmid of claim 12, wherein said gram-positive rolling circle origin of replication comprises the sequence of SEQ ID NO:30.

14. The plasmid of any of claims 10-13, wherein said functional unit is a replicon.

15. The plasmid of claim 14, wherein said replicon is a broad host-range replicon.

16. The plasmid of claim 15, wherein said broad host range replicon is selected from the group consisting of: an RK2 replicon, a pRO1600 replicon, and a p15a/ColE1 replicon.

17. The plasmid of claim 14, wherein said replicon is functional in an organism selected from the genera consisting of: Acetobacter, Achromobacter, Acinetobacter, Aeromonas, Agrobacterium, Alcaligenes, Anabaena, Anaerocellum, Azospirrillum, Azotobacter, Bartonella, Bordetella, Caldicellulosiruptor, Caulobacter, Clavobacter, Clostridium, Enterobacteriaceae, Haemophilus, Hypomycrobium, Legionella, Klebsiella, Methylophilus, Methylosinus, Myxococcus, Neisseria, Paracoccus, Proteus, Pseudomonas, Rhizobium, Rhodopseudomonas, Rhodospirillum, Salmonella, Serratia, Thermoanaerobacter, Thermoanaerobacterium, Thermobacteroides, Thiobacillus, Vibrio, Xanthomonas, Yersinia, and Zymomonas.

18. The plasmid of claim 14, wherein said replicon is a yeast replicon.

19. The plasmid of claim 10, wherein said yeast replicon is CEN6/ARSH.

20. The plasmid of any of claims 10-19, wherein said at least one functional unit encodes a selectable marker.

21. The plasmid of claim 20, wherein said selectable marker confers resistance to an antibiotic selected from the group consisting of: ampicillin, kanamycin, erythromycin, chloramphenicol, gentamycin, kasugamycin, rifampicin, spectinomycin, D-Cycloserine, nalidixic acid, streptomycin, tetracycline, and a combination thereof.

22. The plasmid of claim 20, wherein the selectable marker is a nutritional marker.

23. The plasmid of claim 20, wherein said selectable marker is a yeast selectable marker.

24. The plasmid of claim 23, wherein said yeast selectable marker is selected from the group consisting of URA3, HIS3, LEU2, TRP1, LYS2 and ADE2.

25. The plasmid of any of claims 10-24, wherein said at least one functional unit is a multiple cloning site.

26. The plasmid of claim 25, wherein said multiple cloning site comprises one or more restriction sites selected from the group consisting of: HindIII, MluI, SpeI, BglII, StuI, BspDI/ClaI, PvuII, NdeI, NcoI, SmaI/XmaI, SacII, PvuI, EagI/XmaIII, PaeR7I/XhoI, PstI, EcoRI, SqacI, EcoRV, SphI, NaeI, NheI, BamHI, NazI, ApaI, Acc65L/KpnI, SalI, ApaLI, HpaI, BspEI, NruI, XbaI, BclI, BalI, SwaI, Sse8387I, SrfI, NotI, AscI, PacI, and PmeI, and a combination thereof.

27. The plasmid of claim 26, wherein said multiple cloning site comprises one or more restriction sites selected from the group consisting of: EcoRI, SacI, KpnI, SmaI, XmaI, BamHI, XbaI, HindII, PstI, SphI, HindIII, AvaI, and a combination thereof.

28. The plasmid of any of claims 10-27, wherein said at least one functional unit comprises a sequence that encodes a protein or functional protein fragment.

29. The plasmid of claim 28, wherein said protein or functional fragment thereof facilitates the anaerobic oxidation of an organic compound.

30. The plasmid of claim 28, wherein said protein or functional protein fragment is an enzyme.

31. The plasmid of claim 30, wherein said enzyme is a saccharolytic enzyme or a fermentation enzyme.

32. The plasmid of any of claims 8-31, further comprising a sequence that encodes a reporter gene.

33. The plasmid of claim 32, wherein said reporter gene encodes a protein that is functional in anaerobic conditions.

34. The plasmid of claim 33, wherein said reporter gene is catechol 2,3-oxygenase (xylE).

35. The plasmid of claim 32, wherein said reporter gene is selected from the group consisting of: β-galactosidase, β-glucuronidase, luciferase, green fluorescent protein, and red fluorescent protein.

36. The plasmid of any of claims 32-35, wherein said reporter gene is operably linked to a promoter.

37. The plasmid of claim 36, wherein said promoter is a heterologous promoter.

38. The plasmid of any of claims 8-37, wherein said plasmid further comprises a selectable marker.

39. The plasmid of any of claims 8-38, wherein said plasmid further comprises a sequence encoding a protein or a functional protein fragment.

40. The plasmid of any of claims 8-39, wherein said plasmid further comprises a restriction site.

41. The plasmid of any of claims 8-39, wherein said plasmid further comprises a multiple cloning site.

42. The plasmid of any of claims 8-41, wherein said plasmid is capable of replicating in a yeast host cell.

43. The plasmid of any of claims 8-42, wherein said plasmid is capable of replicating in a yeast host cell and an E. coli host cell.

44. The plasmid of any of claims 8-43, wherein said plasmid is capable of replicating in a yeast host cell, an E. coli host cell, and a thermophilic bacterium host cell.

45. The plasmid of any of claims 8-44, wherein said plasmid is a shuttle vector.

46. The plasmid of claim 45, wherein said shuttle vector is an E. coli-S. cerevisiae-thermophile shuttle vector.

47. The plasmid of claim 46, wherein said E. coli-S. cerevisiae-thermophile shuttle vector comprises a gram-positive rolling circle origin of replication, an antibiotic-resistance gene, a yeast selectable marker, and a yeast replicon.

48. The plasmid of claim 46, wherein said E. coli-S. cerevisiae-thermophile shuttle vector comprises a selectable marker for a thermophilic bacterium.

49. The plasmid of claim 48, wherein said thermophilic bacterium is selected from the group consisting of a Thermoanaerobacterium species, Clostridium species, Thermoanaerobacter species, Thermobacteroides species, Anaerocellum species, and Caldicellulosiruptor species.

50. The plasmid of any of claims 8-49, wherein said plasmid comprises a nucleotide sequence that is at least 90% identical to the sequence of SEQ ID NO:10 or to the sequence of the plasmid deposited as ATCC Deposit No. ______.

51. The plasmid of any of claims 8-49, wherein said plasmid comprises a nucleotide sequence that is at least 90% identical to the sequence of SEQ ID NO:11 or to the sequence of ATCC Deposit No. ______.

52. The plasmid of any of claims 8-49, wherein said plasmid comprises a nucleotide sequence that is at least 90% identical to the sequence of SEQ ID NO:14/

53. The plasmid of any of claims 8-49, wherein said plasmid comprises a nucleotide sequence that is at least 90% identical to the sequence of SEQ ID NO:17.

54. The plasmid of any of claims 8-49, wherein said plasmid comprises a nucleotide sequence that is at least 90% identical to the sequence of SEQ ID NO:20.

55. The plasmid of any of claims 8-49, wherein said plasmid comprises a nucleotide sequence that is at least 90% identical to the sequence of SEQ ID NO:25.

56. The plasmid of any of claims 8-49, wherein said plasmid comprises a nucleotide sequence that is at least 90% identical to the sequence of SEQ ID NO:28.

57. The plasmid of any of claims 8-49, wherein said plasmid comprises a nucleotide sequence that is at least 90% identical to the sequence of SEQ ID NO:39.

58. The plasmid of any of claims 8-49, wherein said plasmid comprises a nucleotide sequence that is at least 90% identical to the sequence of SEQ ID NO:40.

59. A host cell comprising the plasmid of any of claims 8-58.

60. The host cell of claim 59, wherein said host cell is a bacterium.

61. The host cell of claim 59, wherein said bacterium is a thermophilic bacterium.

62. The host cell of claim 61, wherein said thermophilic bacterium is selected from the group consisting of a Thermoanaerobacterium species, Clostridium species, Thermoanaerobacter species, Thermobacteroides species, Anaerocellum species, and Caldicellulosiruptor species.

63. The host cell of claim 59, wherein said host cell is a yeast cell.

64. The host cell of claim 63, wherein said yeast cell is a thermophilic yeast cell.

65. A method for expressing a heterologous sequence encoding a protein or functional protein fragment in a thermophilic host, said method comprising:
(a) transforming a thermophilic host with the plasmid of any of claims 8-58; and
(b) culturing the transformed thermophilic host of (a) for a length of time and under conditions whereby the sequence encoding a protein or a functional protein fragment is expressed.

66. A method for propagating a plasmid in a thermophilic host, said method comprising:
(a) transforming a thermophilic host with the plasmid of any of claims 8-58; and
(b) culturing the transformed thermophilic host of (a) for a length of time and under conditions whereby the plasmid replicates.

67. A method of producing a replicative, thermostable plasmid, said method comprising:
(a) obtaining an isolated nucleotide sequence according to claim 1;
(b) obtaining at least one nucleotide sequence encoding at least one functional unit; and
(c) combining the nucleotide sequences of (a) and (b) together.

68. The method of claim 67, wherein said method further comprises:
(d) obtaining a nucleotide sequence comprising the on sequence of SEQ ID NO:30; and
(e) combining the nucleotide sequences of (a), (b), and (d) together.

69. A plasmid produced by the method of claim 67 or 68.

70. A method of producing a shuttle vector, said method comprising:
(a) providing a first replicon that is autonomously replicable in a first host, said replicon comprising a nucleotide sequence encoding a polypeptide having Rep protein activity, wherein said polypeptide is at least 90% identical to the amino acid sequence of SEQ ID NO:22;
(b) digesting the first replicon with one or more restriction enzymes to obtain a fragment of said first replicon comprising at least the nucleotide sequence encoding a polypeptide having Rep protein activity;
(c) digesting a second replicon that is heterologous to said first replicon and autonomously replicable in a second host with one or more restriction enzymes to obtain a fragment of said second replicon comprising at least an origin of replication; and
(d) ligating said fragments to obtain a shuttle vector that is autonomously replicable in both said first host and said second host.

71. A method of producing a shuttle vector, said method comprising:
(a) providing a first replicon that is autonomously replicable in a first host, said replicon comprising a nucleotide sequence encoding a polypeptide having Rep protein activity, wherein said polypeptide sequence is at least 90% identical to the amino acid sequence of SEQ ID NO:22;
(b) digesting the first replicon with one or more restriction enzymes to obtain a fragment of said first replicon comprising at least the nucleotide sequence encoding a polypeptide having Rep protein activity;
(c) digesting a second replicon that is heterologous to said first replicon and autonomously replicable in a second host with one or more restriction enzymes to obtain a fragment of said second replicon comprising at least an origin of replication;
(d) digesting a third replicon that is heterologous to said first replicon and to said second replicon and that is autonomously replicable in a third host with one or more restriction enzymes to obtain a fragment of said third replicon comprising at least an origin of replication; and
(d) ligating said fragments to obtain a shuttle vector that is autonomously replicable in said first host, said second host and said third host.

72. A method of introducing a functional unit into a shuttle vector, said method comprising:
(a) providing the shuttle vector produced by claim 67 or 68;
(b) digesting said shuttle vector with one or more restriction enzymes;
(c) obtaining a functional unit capable of ligation with said shuttle vector, and
(d) ligating said functional unit to said shuttle vector.

73. The method of any of claims 67-69, wherein said fragment of said first replicon, said fragment of said second replicon, said fragment of said third replicon or said fragment comprising a functional unit is obtained by polymerase chain reaction (PCR) or oligonucleotide synthesis.

74. A shuttle vector produced by the method of any of claims 67-70.

75. A method of propagating a shuttle vector, said method comprising:
(a) transforming a first host cell with the plasmid of any of claim 8-58 or 69, or the shuttle vector of claim 74;
(b) culturing the transformed host cell of (a) for a length of time and under conditions whereby the plasmid or shuttle vector replicates;
(c) isolating the plasmid or shuttle vector of (b); and
(d) transforming a second host cell of a different species than said first host cell with said plasmid or shuttle vector.

76. The method of claim 75, wherein said plasmid or shuttle vector comprises a heterologous sequence encoding a protein or functional fragment thereof.

77. The method of claim 76, wherein said method comprises expressing said heterologous sequence in said first host cell.

78. The method of claim 76, wherein said method comprises expressing said heterologous sequence in said second host cell.

79. An isolated polypeptide comprising a sequence that is at least about 90% identical to SEQ ID NO:22 or a functional fragment thereof.

80. The isolated polypeptide of claim 79, wherein said polypeptide comprises a sequence that is at least about 95% identical to SEQ ID NO:22.

81. The isolated polypeptide of claim 79, wherein said polypeptide comprises a sequence that is at least about 99% identical to SEQ ID NO:22.

82. The isolated polypeptide of claim 79, wherein said polypeptide comprises SEQ ID NO:22.

83. The isolated polypeptide of claim 79, wherein said functional fragment has DNA nicking activity.

84. The isolated polypeptide of claim 79, wherein said functional fragment has specific origin site recognition activity.


SEQUENCE LISTING (40 sequences)

SEQ ID NO: 1 - DNA, 60 bp, Artificial sequence
  Synthetic putative clone containing fragments of pB6A
gattataaagagaagggactttatttattaagtagtatggatgattttattaaaattatg

SEQ ID NO: 2 - DNA, 235 bp, Artificial sequence
  Putative clone containing fragments of pB6A
attgttagcactgggcctggtctgcggcccagacccgaaattatgcaaaataaaatgatattttattatgattatgaacatctctaaaagccttaaaaattgtaaaagaattaaaagaaaaataaaaaccacacgttttgttaaactcatcaaaaaaaactaattcagaaactttgatagaagggaataaatcaacactaaaatctaaacttaaagatttcctattaaattttct

SEQ ID NO: 3 - DNA, 19 bp, Artificial sequence
  Synthetic primer used in sequence reaction with pB6A as the template
cagaaactttgatagaagg

SEQ ID NO: 4 - DNA, 21 bp, Artificial sequence
  Synthetic primer used in sequence reaction with pB6A as the template
cagaccaggcccagtgctaac

SEQ ID NO: 5 - DNA, 25 bp, Artificial sequence
  Synthetic primer used in sequence reaction with pB6A as the template
ggactttatttattaagtagtatgg

SEQ ID NO: 6 - DNA, 2085 bp, Artificial sequence
  Synthetic assembler pBGA sequence
taaagatttatattttgcattacataatgttcatcagatacaaggctatggttgtttatataatataagagatgatactcaattagatttaaaggttgatgacatttataatgatttgattgatttattacaagttacagaaaatcctatacagtctatggaaactgtacaggatttattaaaggatactgaatatacaataataagccgtaagcgtatatttaagtatctaacacaattatatcataaggattgatatttataccgtctgtcggactcatgcggagggggacttgagggggtctcccctcgcattgtacgacagacggtattattattatacaaattttttttatgtaattttttttgtgtaatttttttatacaaataatatttcaattgacaaagttttctatttgtgttaacattgtttatataatagtgaacagtgttaagattaaatgtgaggtgtttgtatggatattaatgattataaagagaagggactttatttattaagtagtatggatgattttattaaaattaatgatttgtttatgggtaaagttgtttctcctggctatgttgcttcggtttttggtgtttccaggtctactgttacacaatggattcaaagacgtaaaattagagcttttaagtataaaggtaaggaaggtgactatatggttatacctattgctgatattattgattacaaaagattgagtaataatgattttatttatgataagttagtgaggtgatttattttatgtttgacgatagctatgttgttaatgagtgttcgtctaatgttagtgaaaatgatagagatttttgtagtttggttggtcgttttatgattattaatggtatagataagttggttattaagattaatagaaaatttaataggaaatctttaagtttagattttagtgttgatttattcccttctatcaaagtttctgaattagttttttttgatgagtttaacaaaacgtgtggtttttatttttcttttaattcttttacaatttttaaggcttttagagatgttcataatcataataaaatatcattttattttgcataatttcgggtctgggccgcagaccaggcccagtgctaacaatattaatttttaatgttaggaattgtttaattcttaattgtgtttttaaaggtagaataattacccattcgccctttagccaacaaaaattaaggaggtataaacatggataaaatggatttgattcttcaagatgaaagactgggtgagatatttaaagatatagatttaacagataatgaaaagagatatcttaaatggttatggaaatgggattatgaaacacgtgatacttttgtatcaattttttttgaagctaaaaaatggtggaaaatgattttttttcttatcttgatatattagaaaaaagcgtactcacgaagtaagaatttgtaaaaaaagaaggggggatttttttggatgagagtttgtacaagcagattttaagtaatattattattactcgtgattattgtaaaaatgttttagataatataaagttcaatgaaaaaataattgattattatgttatgttacaaaatgatgtttttattgattttactaataaaataaattcaataagggattgtaataaatattggtatttggatgtttataaaaagcagaaaataaaggattttaaaaagactaatttgtgtaaagataagttctgtaataattgtaagaaagttaaacaggcttcaagaatgcaaaaatatattcctgaattacagaaatacaaagatggcttatatcattttatatttactgttgaaaatgtgccaggtagtgaattaagagatactattgataggttgtttaagtcttttaagtcatttacaaggtatttaagtggtaatcttaaaataaaaggtgttaattttgataaatggggttataaaggctgtgtaaggtctttagaggtaacttatagtatgattgataatcatattatgtatcatccacacttgcatgttgcgatgatattagatcctttttacgatgggtta

SEQ ID NO: 7 - DNA, 20 bp, Artificial sequence
  Synthetic primer used in sequence reaction with pB6A as the template
cctgtacagtttccatagac

SEQ ID NO: 8 - DNA, 21 bp, Artificial sequence
  Synthetic primer used in sequence reaction with pB6A as the template
ggttataaaggctgtgtaagg

SEQ ID NO: 9 - DNA, 2349 bp, Thermoanaerobacterium Saccharolyticum
ggtgttaattttgataaatggggttataaaggctgtgtaaggtctttagaggtaacttatagtatgattgataatcatattatgtatcatccacacttgcatgttgcgatgatattagatcctttttacgatggttttaatgttgaaaggatgcatataattaataagtttagttatagctatggtgttttaaaaaggttgtttactgatgatgaattattaattcaaaaaatttggtatttattgtttaataatattgaggttaacatggccaatataaataatttagaggatggttattcttgtttagttaataagtttagtgattatgattatgcggagctgtttaagtatatttgtaaaaatactgatgaacaaggtttacttatgacttatgatatttttaaagatttatattttgcattacataatgttcatcagatacaaggctatggttgtttatataatataagagatgatactcaattagatttaaaggttgatgacatttataatgatttgattgatttattacaagttacagaaaatcctatacagtctatggaaactgtacaggatttattaaaggatactgaatatacaataataagccgtaagcgtatatttaagtatctaacacaattatatcataaggattgatatttataccgtctgtcggactcatgcggagggggacttgagggggtctcccctcgcattgtacgacagacggtattattattatacaaattttttttatgtaattttttttgtgtaatttttttatacaaataatatttcaattgacaaagttttctatttgtgttaacattgtttatataatagtgaacagtgttaagattaaatgtgaggtgtttgtatggatattaatgattataaagagaagggactttatttattaagtagtatggatgattttattaaaattaatgatttgtttatgggtaaagttgtttctcctggctatgttgcttcggtttttggtgtttccaggtctactgttacacaatggattcaaagacgtaaaattagagcttttaagtataaaggtaaggaaggtgactatatggttatacctattgctgatattattgattacaaaagattgagtaataatgattttatttatgataagttagtgaggtgatttattttatgtttgacgatagctatgttgttaatgagtgttcgtctaatgttagtgaaaatgatagagatttttgtagtttggttggtcgttttatgattattaatggtatagataagttggttattaagattaatagaaaatttaataggaaatctttaagtttagattttagtgttgatttattcccttctatcaaagtttctgaattagttttttttgatgagtttaacaaaacgtgtggtttttatttttcttttaattcttttacaatttttaaggcttttagagatgttcataatcataataaaatatcattttattttgcataatttcgggtctgggccgcagaccaggcccagtgctaacaatattaatttttaatgttaggaattgtttaattcttaattgtgtttttaaaggtagaataattacccattcgccctttagccaacaaaaattaaggaggtataaacatggataaaatggatttgattcttcaagatgaaagactgggtgagatatttaaagatatagatttaacagataatgaaaagagatatcttaaatggttatggaaatgggattatgaaacacgtgatacttttgtatcaatttttttgaagctaaaaaatggtggaaaatgatttttttcttatcttgatatattagaaaaaagcgtactcacgaagtaagaatttgtaaaaaaagaaggggggatttttttggatgagagtttgtacaagcagattttaagtaatattattattactcgtgattattgtaaaaatgttttagataatataaagttcaatgaaaaaataattgattattatgttatgttacaaaatgatgtttttattgattttactaataaaataaattcaataagggattgtaataaatattggtatttggatgtttataaaaagcagaaaataaaggattttaaaaagactaatttgtgtaaagataagttctgtaataattgtaagaaagttaaacaggcttcaagaatgcaaaaatatattcctgaattacagaaatacaaagatggcttatatcattttatatttactgttgaaaatgtgccaggtagtgaattaagagatactattgataggttgtttaagtcttttaagtcatttacaaggtatttaagtggtaatcttaaaataaaa

SEQ ID NO: 10 - DNA, 5035 bp, Artificial sequence
  Synthetic plasmid pMU121
aattgacaaagttttctatttgtgttaacattgtttatataatagtgaacagtgttaagattaaatgtgaggtgtttgtatggatattaatgattataaagagaagggactttatttattaagtagtatggatgattttattaaaattaatgatttgtttatgggtaaagttgtttctcctggctatgttgcttcggtttttggtgtttccaggtctactgttacacaatggattcaaagacgtaaaattagagcttttaagtataaaggtaaggaaggtgactatatggttatacctattgctgatattattgattacaaaagattgagtaataatgattttatttatgataagttagtgaggtgatttattttatgtttgacgatagctatgttgttaatgagtgttcgtctaatgttagtgaaaatgatagagatttttgtagtttggttggtcgttttatgattattaatggtatagataagttggttattaagattaatagaaaatttaataggaaatctttaagtttagattttagtgttgatttattcccttctatcaaagtttctgaattagttttttttgatgagtttaacaaaacgtgtggtttttatttttcttttaattcttttacaatttttaaggcttttagagatgttcataatcataataaaatatcattttattttgcataatttcgggtctgggccgcagaccaggcccagtgctaacaatattaatttttaatgttaggaattgtttaattcttaattgtgtttttaaaggtagaataattacccattcgccctttagccaacaaaaattaaggaggtataaacatggataaaatggatttgattcttcaagatgaaagactgggtgagatatttaaagatatagatttaacagataatgaaaagagatatcttaaatggttatggaaatgggattatgaaacacgtgatacttttgtatcaatttttttgaagctaaaaaatggtggaaaatgatttttttcttatcttgatatattagaaaaaagcgtactcacgaagtaagaatttgtaaaaaaagaaggggggatttttttggatgagagtttgtacaagcagattttaagtaatattattattactcgtgattattgtaaaaatgttttagataatataaagttcaatgaaaaaataattgattattatgttatgttacaaaatgatgtttttattgattttactaataaaataaattcaataagggattgtaataaatattggtatttggatgtttataaaaagcagaaaataaaggattttaaaaagactaatttgtgtaaagataagttctgtaataattgtaagaaagttaaacaggcttcaagaatgcaaaaatatattcctgaattacagaaatacaaagatggcttatatcattttatatttactgttgaaaatgtgccaggtagtgaattaagagatactattgataggttgtttaagtcttttaagtcatttacaaggtatttaagtggtaatcttaaaataaaaggtgttaattttgataaatggggttataaaggctgtgtaaggtctttagaggtaacttatagtatgattgataatcatattatgtatcatccacacttgcatgttgcgatgatattagatcctttttacgatggttttaatgttgaaaggatgcatataattaataagtttagttatagctatggtgttttaaaaaggttgtttactgatgatgaattattaattcaaaaaatttggtatttattgtttaataatattgaggttaacatggccaatataaataatttagaggatggttattcttgtttagttaataagtttagtgattatgattatgcggagctgtttaagtatatttgtaaaaatactgatgaacaaggtttacttatgacttatgatatttttaaagatttatattttgcattacataatgttcatcagatacaaggctatggttgtttatataatataagagatgatactcaattagatttaaaggttgatgacatttataatgatttgattgatttattacaagttacagaaaatcctatacagtctatggaaactgtacaggatttattaaaggatactgaatatacaataataagccgtaagcgtatatttaagtatctaacacaattatatcataaggattgatatttataccgtctgtcggactcatgcggagggggacttgagggggtctcccctcgcattgtacgacagacggtattattattatacaaattttttttatgtaattttttttgtgtaatttttttatacaaataatatttcaattcgagctcggtacccggggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtg

SEQ ID NO: 11 - DNA, 6403 bp, Artificial sequence
  Synthetic plasmid pMU131 with a Kanamycin marker
aattgacaaagttttctatttgtgttaacattgtttatataatagtgaacagtgttaagattaaatgtgaggtgtttgtatggatattaatgattataaagagaagggactttatttattaagtagtatggatgattttattaaaattaatgatttgtttatgggtaaagttgtttctcctggctatgttgcttcggtttttggtgtttccaggtctactgttacacaatggattcaaagacgtaaaattagagcttttaagtataaaggtaaggaaggtgactatatggttatacctattgctgatattattgattacaaaagattgagtaataatgattttatttatgataagttagtgaggtgatttattttatgtttgacgatagctatgttgttaatgagtgttcgtctaatgttagtgaaaatgatagagatttttgtagtttggttggtcgttttatgattattaatggtatagataagttggttattaagattaatagaaaatttaataggaaatctttaagtttagattttagtgttgatttattcccttctatcaaagtttctgaattagttttttttgatgagtttaacaaaacgtgtggtttttatttttcttttaattcttttacaatttttaaggcttttagagatgttcataatcataataaaatatcattttattttgcataatttcgggtctgggccgcagaccaggcccagtgctaacaatattaatttttaatgttaggaattgtttaattcttaattgtgtttttaaaggtagaataattacccattcgccctttagccaacaaaaattaaggaggtataaacatggataaaatggatttgattcttcaagatgaaagactgggtgagatatttaaagatatagatttaacagataatgaaaagagatatcttaaatggttatggaaatgggattatgaaacacgtgatacttttgtatcaatttttttgaagctaaaaaatggtggaaaatgatttttttcttatcttgatatattagaaaaaagcgtactcacgaagtaagaatttgtaaaaaaagaaggggggatttttttggatgagagtttgtacaagcagattttaagtaatattattattactcgtgattattgtaaaaatgttttagataatataaagttcaatgaaaaaataattgattattatgttatgttacaaaatgatgtttttattgattttactaataaaataaattcaataagggattgtaataaatattggtatttggatgtttataaaaagcagaaaataaaggattttaaaaagactaatttgtgtaaagataagttctgtaataattgtaagaaagttaaacaggcttcaagaatgcaaaaatatattcctgaattacagaaatacaaagatggcttatatcattttatatttactgttgaaaatgtgccaggtagtgaattaagagatactattgataggttgtttaagtcttttaagtcatttacaaggtatttaagtggtaatcttaaaataaaaggtgttaattttgataaatggggttataaaggctgtgtaaggtctttagaggtaacttatagtatgattgataatcatattatgtatcatccacacttgcatgttgcgatgatattagatcctttttacgatggttttaatgttgaaaggatgcatataattaataagtttagttatagctatggtgttttaaaaaggttgtttactgatgatgaattattaattcaaaaaatttggtatttattgtttaataatattgaggttaacatggccaatataaataatttagaggatggttattcttgtttagttaataagtttagtgattatgattatgcggagctgtttaagtatatttgtaaaaatactgatgaacaaggtttacttatgacttatgatatttttaaagatttatattttgcattacataatgttcatcagatacaaggctatggttgtttatataatataagagatgatactcaattagatttaaaggttgatgacatttataatgatttgattgatttattacaagttacagaaaatcctatacagtctatggaaactgtacaggatttattaaaggatactgaatatacaataataagccgtaagcgtatatttaagtatctaacacaattatatcataaggattgatatttataccgtctgtcggactcatgcggagggggacttgagggggtctcccctcgcattgtacgacagacggtattattattatacaaattttttttatgtaattttttttgtgtaatttttttatacaaataatatttcaattcgagctcggtacccggggatcctctagagtcgacctgcaggcatgcaagcttggctgcaggtcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgtggtttcaaaatcggctccgtcgatactatgttatacgccaactttcaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatttagatgtctaaaaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtg

SEQ ID NO: 12 - DNA, 31 bp, Artificial sequence
  Synthetic primer used to amplify chloramphenicol and erythromycin resistance genes
ggcgaagcttggtctttgtactaacctgtgg

SEQ ID NO: 13 - DNA, 30 bp, Artificial sequence
  Synthetic primer used to amplify chloramphenicol and erythromycin resistance genes
ggcgaagcttgagttagctcactcattagg

SEQ ID NO: 14 - DNA, 7106 bp, Artificial sequence
  Synthetic plasmid PMU141
aattgacaaagttttctatttgtgttaacattgtttatataatagtgaacagtgttaagattaaatgtgaggtgtttgtatggatattaatgattataaagagaagggactttatttattaagtagtatggatgattttattaaaattaatgatttgtttatgggtaaagttgtttctcctggctatgttgcttcggtttttggtgtttccaggtctactgttacacaatggattcaaagacgtaaaattagagcttttaagtataaaggtaaggaaggtgactatatggttatacctattgctgatattattgattacaaaagattgagtaataatgattttatttatgataagttagtgaggtgatttattttatgtttgacgatagctatgttgttaatgagtgttcgtctaatgttagtgaaaatgatagagatttttgtagtttggttggtcgttttatgattattaatggtatagataagttggttattaagattaatagaaaatttaataggaaatctttaagtttagattttagtgttgatttattcccttctatcaaagtttctgaattagttttttttgatgagtttaacaaaacgtgtggtttttatttttcttttaattcttttacaatttttaaggcttttagagatgttcataatcataataaaatatcattttattttgcataatttcgggtctgggccgcagaccaggcccagtgctaacaatattaatttttaatgttaggaattgtttaattcttaattgtgtttttaaaggtagaataattacccattcgccctttagccaacaaaaattaaggaggtataaacatggataaaatggatttgattcttcaagatgaaagactgggtgagatatttaaagatatagatttaacagataatgaaaagagatatcttaaatggttatggaaatgggattatgaaacacgtgatacttttgtatcaatttttttgaagctaaaaaatggtggaaaatgatttttttcttatcttgatatattagaaaaaagcgtactcacgaagtaagaatttgtaaaaaaagaaggggggatttttttggatgagagtttgtacaagcagattttaagtaatattattattactcgtgattattgtaaaaatgttttagataatataaagttcaatgaaaaaataattgattattatgttatgttacaaaatgatgtttttattgattttactaataaaataaattcaataagggattgtaataaatattggtatttggatgtttataaaaagcagaaaataaaggattttaaaaagactaatttgtgtaaagataagttctgtaataattgtaagaaagttaaacaggcttcaagaatgcaaaaatatattcctgaattacagaaatacaaagatggcttatatcattttatatttactgttgaaaatgtgccaggtagtgaattaagagatactattgataggttgtttaagtcttttaagtcatttacaaggtatttaagtggtaatcttaaaataaaaggtgttaattttgataaatggggttataaaggctgtgtaaggtctttagaggtaacttatagtatgattgataatcatattatgtatcatccacacttgcatgttgcgatgatattagatcctttttacgatggttttaatgttgaaaggatgcatataattaataagtttagttatagctatggtgttttaaaaaggttgtttactgatgatgaattattaattcaaaaaatttggtatttattgtttaataatattgaggttaacatggccaatataaataatttagaggatggttattcttgtttagttaataagtttagtgattatgattatgcggagctgtttaagtatatttgtaaaaatactgatgaacaaggtttacttatgacttatgatatttttaaagatttatattttgcattacataatgttcatcagatacaaggctatggttgtttatataatataagagatgatactcaattagatttaaaggttgatgacatttataatgatttgattgatttattacaagttacagaaaatcctatacagtctatggaaactgtacaggatttattaaaggatactgaatatacaataataagccgtaagcgtatatttaagtatctaacacaattatatcataaggattgatatttataccgtctgtcggactcatgcggagggggacttgagggggtctcccctcgcattgtacgacagacggtattattattatacaaattttttttatgtaattttttttgtgtaatttttttatacaaataatatttcaattcgagctcggtacccggggatcctctagagtcgacctgcaggcatgcaagcttgttatgtataaaattgtagattttagggtaacaaaaaacaccgtatttctacgatgtttttgcttaaatacttgtttttagttacagacaaacctgaagttaactatttatcaattcctgcaattcgtttacaaaacggcaaatgtgaaatccgtcacatactgcgtgatgaacttgaattgccaaaggaagtataattttgttatcttctttataatatttccccatagtaaaaataggaatcaaataatcatatcctttctgcaaattcagattaaagccatcgaaggttgaccacggtatcatagatacattaaaaatgttttccggagcatttggctttccttccattctatgattgtttccataccgttgcgtatcactttcataatctgctaaaaatgatttaaagtcagacttacactcagtccaaaggctggaaaatgtttcagtatcattgtgaaatattgtatagcttggtatcatctcatcatatatccccaattcaccatcttgattgattgccgtcctaaactctgaatggcggtttacaatcattgcaatataataaagcattgcaggatatagtttcattcccttttcctttatttgtgtgatatccactttaacggtcatgctgtatgtacaaggtacacttgcaaagtagtggtcaaaatactcttttctgttccaactatttttatcaattttttcaaataccatctaagttccctctcaaattcaagtttatcgctctaatgaacaaagatattataccacatttttgtgaatttttcaacttgcccacttcgactgcactcccgacttaataacttcttgaacacttgccgaaaaagaaaaactgccgggtacgtacccgggatcgatccccgccgagcgcttagtgggaatttgtaccccttatcgatacaaattccccgtaggcgctagggacctctttagctccttggaagctgtcagtagtatacctaataatttatctacattccctttagtaacgtgtaactttccaaatttacaaaagcgactcatagaattatttcctcccgttaaataatagataactattaaaaatagacaatacttgctcataagtaacggtacttaaattgtttactttggcgtgtttcattgcttgatgaaactgatttttagtaaacagttgacgatattctcgattgacccattttgaaacaaagtacgtatatagcttccaatatttatctggaacatctgtggtatggcgggtaagttttattaagacactgtttacttttggtttaggatgaaagcattccgctggcagcttaagcaattgctgaatcgagacttgagtgtgcaagagcaaccctagtgttcggtgaatatccaaggtacgcttgtagaatccttcttcaacaatcagatagatgtcagacgcatggctttcaaaaaccacttttttaataatttgtgtgcttaaatggtaaggaatactcccaacaattttatacctctgtttgttagggaattgaaactgtagaatatcttggtgaattaaagtgacacgagtattcagttttaatttttctgacgataagttgaatagatgactgtctaattcaatagacgttacctgtttacttattttagccagtttcgtcgttaaatgccctttacctgttccaatttcgtaaacggtatcggtttcttttaaattcaattgttttattatttggttgagtactttttcactcgttaaaaagttttgagaatattttatatttttgttcatgtaatcactccttcttaattacaaatttttagcatctaatttaacttcaattcctattatacaaaattttaagatactgcactatcaacacactcttaagtttgcttctaagtcttatttccataacttcttttacgtttccgggtacaattcgtaatcatgtcatagctgtttcctgtgtgaaattcttatccgctcacaattccacacaacatacgagccggaagcataaagtgtaaagcctggggtgcctaatgagtgagctaactcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtg

SEQ ID NO: 15 - DNA, 31 bp, Artificial sequence; identical to SEQ ID NO: 12
  Synthetic primer used to amplify chloramphenicol resistance gene

SEQ ID NO: 16 - DNA, 31 bp, Artificial sequence
  Synthetic primer used to amplify chloramphenicol resistance gene
ggcgaagcttctactgacagcttccaaggag

SEQ ID NO: 17 - DNA, 6045 bp, Artificial sequence
  Synthetic plasmid pMU144
aattgacaaagttttctatttgtgttaacattgtttatataatagtgaacagtgttaagattaaatgtgaggtgtttgtatggatattaatgattataaagagaagggactttatttattaagtagtatggatgattttattaaaattaatgatttgtttatgggtaaagttgtttctcctggctatgttgcttcggtttttggtgtttccaggtctactgttacacaatggattcaaagacgtaaaattagagcttttaagtataaaggtaaggaaggtgactatatggttatacctattgctgatattattgattacaaaagattgagtaataatgattttatttatgataagttagtgaggtgatttattttatgtttgacgatagctatgttgttaatgagtgttcgtctaatgttagtgaaaatgatagagatttttgtagtttggttggtcgttttatgattattaatggtatagataagttggttattaagattaatagaaaatttaataggaaatctttaagtttagattttagtgttgatttattcccttctatcaaagtttctgaattagttttttttgatgagtttaacaaaacgtgtggtttttatttttcttttaattcttttacaatttttaaggcttttagagatgttcataatcataataaaatatcattttattttgcataatttcgggtctgggccgcagaccaggcccagtgctaacaatattaatttttaatgttaggaattgtttaattcttaattgtgtttttaaaggtagaataattacccattcgccctttagccaacaaaaattaaggaggtataaacatggataaaatggatttgattcttcaagatgaaagactgggtgagatatttaaagatatagatttaacagataatgaaaagagatatcttaaatggttatggaaatgggattatgaaacacgtgatacttttgtatcaatttttttgaagctaaaaaatggtggaaaatgatttttttcttatcttgatatattagaaaaaagcgtactcacgaagtaagaatttgtaaaaaaagaaggggggatttttttggatgagagtttgtacaagcagattttaagtaatattattattactcgtgattattgtaaaaatgttttagataatataaagttcaatgaaaaaataattgattattatgttatgttacaaaatgatgtttttattgattttactaataaaataaattcaataagggattgtaataaatattggtatttggatgtttataaaaagcagaaaataaaggattttaaaaagactaatttgtgtaaagataagttctgtaataattgtaagaaagttaaacaggcttcaagaatgcaaaaatatattcctgaattacagaaatacaaagatggcttatatcattttatatttactgttgaaaatgtgccaggtagtgaattaagagatactattgataggttgtttaagtcttttaagtcatttacaaggtatttaagtggtaatcttaaaataaaaggtgttaattttgataaatggggttataaaggctgtgtaaggtctttagaggtaacttatagtatgattgataatcatattatgtatcatccacacttgcatgttgcgatgatattagatcctttttacgatggttttaatgttgaaaggatgcatataattaataagtttagttatagctatggtgttttaaaaaggttgtttactgatgatgaattattaattcaaaaaatttggtatttattgtttaataatattgaggttaacatggccaatataaataatttagaggatggttattcttgtttagttaataagtttagtgattatgattatgcggagctgtttaagtatatttgtaaaaatactgatgaacaaggtttacttatgacttatgatatttttaaagatttatattttgcattacataatgttcatcagatacaaggctatggttgtttatataatataagagatgatactcaattagatttaaaggttgatgacatttataatgatttgattgatttattacaagttacagaaaatcctatacagtctatggaaactgtacaggatttattaaaggatactgaatatacaataataagccgtaagcgtatatttaagtatctaacacaattatatcataaggattgatatttataccgtctgtcggactcatgcggagggggacttgagggggtctcccctcgcattgtacgacagacggtattattattatacaaattttttttatgtaattttttttgtgtaatttttttatacaaataatatttcaattcgagctcggtacccggggatcctctagagtcgacctgcaggcatgcaagcttggtctttgtactaacctgtggttatgtataaaattgtagattttagggtaacaaaaaacaccgtatttctacgatgtttttgcttaaatacttgtttttagttacagacaaacctgaagttaactatttatcaattcctgcaattcgtttacaaaacggcaaatgtgaaatccgtcacatactgcgtgatgaacttgaattgccaaaggaagtataattttgttatcttctttataatatttccccatagtaaaaataggaatcaaataatcatatcctttctgcaaattcagattaaagccatcgaaggttgaccacggtatcatagatacattaaaaatgttttccggagcatttggctttccttccattctatgattgtttccataccgttgcgtatcactttcataatctgctaaaaatgatttaaagtcagacttacactcagtccaaaggctggaaaatgtttcagtatcattgtgaaatattgtatagcttggtatcatctcatcatatatccccaattcaccatcttgattgattgccgtcctaaactctgaatggcggtttacaatcattgcaatataataaagcattgcaggatatagtttcattcccttttcctttatttgtgtgatatccactttaacggtcatgctgtatgtacaaggtacacttgcaaagtagtggtcaaaatactcttttctgttccaactatttttatcaattttttcaaataccatctaagttccctctcaaattcaagtttatcgctctaatgaacaaagatattataccacatttttgtgaatttttcaacttgcccacttcgactgcactcccgacttaataacttcttgaacacttgccgaaaaagaaaaactgccgggtacgtacccgggatcgatccccgccgagcgcttagtgggaatttgtaccccttatcgatacaaattccccgtaggcgctagggacctctttagctccttggaagctgtcagtagaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtg

SEQ ID NO: 18 - DNA, 31 bp, Artificial sequence
  Synthetic primer used to amplify erythromycin resistance gene
ggcgaagcttctccttggaagctgtcagtag

SEQ ID NO: 19 - DNA, 30 bp, Artificial sequence; identical to SEQ ID NO: 13
  Synthetic primer used to amplify erythromycin resistance gene

SEQ ID NO: 20 - DNA, 6143 bp, Artificial sequence
  Synthetic plasmid pMU143
aattgacaaagttttctatttgtgttaacattgtttatataatagtgaacagtgttaagattaaatgtgaggtgtttgtatggatattaatgattataaagagaagggactttatttattaagtagtatggatgattttattaaaattaatgatttgtttatgggtaaagttgtttctcctggctatgttgcttcggtttttggtgtttccaggtctactgttacacaatggattcaaagacgtaaaattagagcttttaagtataaaggtaaggaaggtgactatatggttatacctattgctgatattattgattacaaaagattgagtaataatgattttatttatgataagttagtgaggtgatttattttatgtttgacgatagctatgttgttaatgagtgttcgtctaatgttagtgaaaatgatagagatttttgtagtttggttggtcgttttatgattattaatggtatagataagttggttattaagattaatagaaaatttaataggaaatctttaagtttagattttagtgttgatttattcccttctatcaaagtttctgaattagttttttttgatgagtttaacaaaacgtgtggtttttatttttcttttaattcttttacaatttttaaggcttttagagatgttcataatcataataaaatatcattttattttgcataatttcgggtctgggccgcagaccaggcccagtgctaacaatattaatttttaatgttaggaattgtttaattcttaattgtgtttttaaaggtagaataattacccattcgccctttagccaacaaaaattaaggaggtataaacatggataaaatggatttgattcttcaagatgaaagactgggtgagatatttaaagatatagatttaacagataatgaaaagagatatcttaaatggttatggaaatgggattatgaaacacgtgatacttttgtatcaatttttttgaagctaaaaaatggtggaaaatgatttttttcttatcttgatatattagaaaaaagcgtactcacgaagtaagaatttgtaaaaaaagaaggggggatttttttggatgagagtttgtacaagcagattttaagtaatattattattactcgtgattattgtaaaaatgttttagataatataaagttcaatgaaaaaataattgattattatgttatgttacaaaatgatgtttttattgattttactaataaaataaattcaataagggattgtaataaatattggtatttggatgtttataaaaagcagaaaataaaggattttaaaaagactaatttgtgtaaagataagttctgtaataattgtaagaaagttaaacaggcttcaagaatgcaaaaatatattcctgaattacagaaatacaaagatggcttatatcattttatatttactgttgaaaatgtgccaggtagtgaattaagagatactattgataggttgtttaagtcttttaagtcatttacaaggtatttaagtggtaatcttaaaataaaaggtgttaattttgataaatggggttataaaggctgtgtaaggtctttagaggtaacttatagtatgattgataatcatattatgtatcatccacacttgcatgttgcgatgatattagatcctttttacgatggttttaatgttgaaaggatgcatataattaataagtttagttatagctatggtgttttaaaaaggttgtttactgatgatgaattattaattcaaaaaatttggtatttattgtttaataatattgaggttaacatggccaatataaataatttagaggatggttattcttgtttagttaataagtttagtgattatgattatgcggagctgtttaagtatatttgtaaaaatactgatgaacaaggtttacttatgacttatgatatttttaaagatttatattttgcattacataatgttcatcagatacaaggctatggttgtttatataatataagagatgatactcaattagatttaaaggttgatgacatttataatgatttgattgatttattacaagttacagaaaatcctatacagtctatggaaactgtacaggatttattaaaggatactgaatatacaataataagccgtaagcgtatatttaagtatctaacacaattatatcataaggattgatatttataccgtctgtcggactcatgcggagggggacttgagggggtctcccctcgcattgtacgacagacggtattattattatacaaattttttttatgtaattttttttgtgtaatttttttatacaaataatatttcaattcgagctcggtacccggggatcctctagagtcgacctgcaggcatgcaagcttctccttggaagctgtcagtagtatacctaataatttatctacattccctttagtaacgtgtaactttccaaatttacaaaagcgactcatagaattatttcctcccgttaaataatagataactattaaaaatagacaatacttgctcataagtaacggtacttaaattgtttactttggcgtgtttcattgcttgatgaaactgatttttagtaaacagttgacgatattctcgattgacccattttgaaacaaagtacgtatatagcttccaatatttatctggaacatctgtggtatggcgggtaagttttattaagacactgtttacttttggtttaggatgaaagcattccgctggcagcttaagcaattgctgaatcgagacttgagtgtgcaagagcaaccctagtgttcggtgaatatccaaggtacgcttgtagaatccttcttcaacaatcagatagatgtcagacgcatggctttcaaaaaccacttttttaataatttgtgtgcttaaatggtaaggaatactcccaacaattttatacctctgtttgttagggaattgaaactgtagaatatcttggtgaattaaagtgacacgagtattcagttttaatttttctgacgataagttgaatagatgactgtctaattcaatagacgttacctgtttacttattttagccagtttcgtcgttaaatgccctttacctgttccaatttcgtaaacggtatcggtttcttttaaattcaattgttttattatttggttgagtactttttcactcgttaaaaagttttgagaatattttatatttttgttcatgtaatcactccttcttaattacaaatttttagcatctaatttaacttcaattcctattatacaaaattttaagatactgcactatcaacacactcttaagtttgcttctaagtcttatttccataacttcttttacgtttccgggtacaattcgtaatcatgtcatagctgtttcctgtgtgaaattcttatccgctcacaattccacacaacatacgagccggaagcataaagtgtaaagcctggggtgcctaatgagtgagctaactcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtg

SEQ ID NO: 21 - DNA, 999 bp, Thermoanaerobacterium Saccharolyticum
atgttacaaaatgatgtttttattgattttactaataaaataaattcaataagggattgtaataaatattggtatttggatgtttataaaaagcagaaaataaaggattttaaaaagactaatttgtgtaaagataagttctgtaataattgtaagaaagttaaacaggcttcaagaatgcaaaaatatattcctgaattacagaaatacaaagatggcttatatcattttatatttactgttgaaaatgtgccaggtagtgaattaagagatactattgataggttgtttaagtcttttaagtcatttacaaggtatttaagtggtaatcttaaaataaaaggtgttaattttgataaatggggttataaaggctgtgtaaggtctttagaggtaacttatagtatgattgataatcatattatgtatcatccacacttgcatgttgcgatgatattagatcctttttacgatggttttaatgttgaaaggatgcatataattaataagtttagttatagctatggtgttttaaaaaggttgtttactgatgatgaattattaattcaaaaaatttggtatttattgtttaataatattgaggttaacatggccaatataaataatttagaggatggttattcttgtttagttaataagtttagtgattatgattatgcggagctgtttaagtatatttgtaaaaatactgatgaacaaggtttacttatgacttatgatatttttaaagatttatattttgcattacataatgttcatcagatacaaggctatggttgtttatataatataagagatgatactcaattagatttaaaggttgatgacatttataatgatttgattgatttattacaagttacagaaaatcctatacagtctatggaaactgtacaggatttattaaaggatactgaatatacaataataagccgtaagcgtatatttaagtatctaacacaattatatcataaggat

SEQ ID NO: 22 - protein, 332 aa, Thermoanaerobacterium Saccharolyticum
MLQNDVFIDFTNKINSIRDCNKYWYLDVYKKQKIKDFKKTNLCKDKFCNNCKKVKQASRMQKYIPELQKYKDGLYHFIFTVENVPGSELRDTIDRLFKSFKSFTRYLSGNLKIKGVNFDKWGYKGCVRSLEVTYSMIDNHIMYHPHLHVAMILDPFYDGFNVERMHIINKFSYSYGVLKRLFTDDELLIQKIWYLLFNNIEVNMANINNLEDGYSCLVNKFSDYDYAELFKYICKNTDEQGLMTYDIFKDLYFALHNVHQIQGYGCLYNIRDDTQLDLKVDDIYNDLIDLLQVTENPIQSMETVQDLLKDTEYTIISRKRIFKYLTQLYHKD

SEQ ID NO: 23 - DNA, 57 bp, Artificial sequence
  Synthetic primer used to amplify Ura3-Cen6/Arsh sequence
ctttccagtcgggaaacctgtcgtgccagcagatctgatcgcttgcctgtaacttac

SEQ ID NO: 24 - DNA, 56 bp, Artificial sequence
  Synthetic primer used to amplify Ura3-Cen6/Arsh sequence
gcctttgagtgagctgataccgctcgccgcagatctcgaaaagtgggtaataactg

SEQ ID NO: 25 - DNA, 6589 bp, Artificial sequence
  Synthetic plasmid pMU158
aattgacaaagttttctatttgtgttaacattgtttatataatagtgaacagtgttaagattaaatgtgaggtgtttgtatggatattaatgattataaagagaagggactttatttattaagtagtatggatgattttattaaaattaatgatttgtttatgggtaaagttgtttctcctggctatgttgcttcggtttttggtgtttccaggtctactgttacacaatggattcaaagacgtaaaattagagcttttaagtataaaggtaaggaaggtgactatatggttatacctattgctgatattattgattacaaaagattgagtaataatgattttatttatgataagttagtgaggtgatttattttatgtttgacgatagctatgttgttaatgagtgttcgtctaatgttagtgaaaatgatagagatttttgtagtttggttggtcgttttatgattattaatggtatagataagttggttattaagattaatagaaaatttaataggaaatctttaagtttagattttagtgttgatttattcccttctatcaaagtttctgaattagttttttttgatgagtttaacaaaacgtgtggtttttatttttcttttaattcttttacaatttttaaggcttttagagatgttcataatcataataaaatatcattttattttgcataatttcgggtctgggccgcagaccaggcccagtgctaacaatattaatttttaatgttaggaattgtttaattcttaattgtgtttttaaaggtagaataattacccattcgccctttagccaacaaaaattaaggaggtataaacatggataaaatggatttgattcttcaagatgaaagactgggtgagatatttaaagatatagatttaacagataatgaaaagagatatcttaaatggttatggaaatgggattatgaaacacgtgatacttttgtatcaatttttttgaagctaaaaaatggtggaaaatgatttttttcttatcttgatatattagaaaaaagcgtactcacgaagtaagaatttgtaaaaaaagaaggggggatttttttggatgagagtttgtacaagcagattttaagtaatattattattactcgtgattattgtaaaaatgttttagataatataaagttcaatgaaaaaataattgattattatgttatgttacaaaatgatgtttttattgattttactaataaaataaattcaataagggattgtaataaatattggtatttggatgtttataaaaagcagaaaataaaggattttaaaaagactaatttgtgtaaagataagttctgtaataattgtaagaaagttaaacaggcttcaagaatgcaaaaatatattcctgaattacagaaatacaaagatggcttatatcattttatatttactgttgaaaatgtgccaggtagtgaattaagagatactattgataggttgtttaagtcttttaagtcatttacaaggtatttaagtggtaatcttaaaataaaaggtgttaattttgataaatggggttataaaggctgtgtaaggtctttagaggtaacttatagtatgattgataatcatattatgtatcatccacacttgcatgttgcgatgatattagatcctttttacgatggttttaatgttgaaaggatgcatataattaataagtttagttatagctatggtgttttaaaaaggttgtttactgatgatgaattattaattcaaaaaatttggtatttattgtttaataatattgaggttaacatggccaatataaataatttagaggatggttattcttgtttagttaataagtttagtgattatgattatgcggagctgtttaagtatatttgtaaaaatactgatgaacaaggtttacttatgacttatgatatttttaaagatttatattttgcattacataatgttcatcagatacaaggctatggttgtttatataatataagagatgatactcaattagatttaaaggttgatgacatttataatgatttgattgatttattacaagttacagaaaatcctatacagtctatggaaactgtacaggatttattaaaggatactgaatatacaataataagccgtaagcgtatatttaagtatctaacacaattatatcataaggattgatatttataccgtctgtcggactcatgcggagggggacttgagggggtctcccctcgcattgtacgacagacggtattattattatacaaattttttttatgtaattttttttgtgtaatttttttatacaaataatatttcaattcgagctcggtacccggggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcagatctgatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggacccatcgataagctagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccacttttcgagatctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtg

SEQ ID NO: 26 - DNA, 50 bp, Artificial sequence
  Synthetic primer used to amplify kanamycin resistance gene
aatgtgcgcggaacccctatttgtttatttaacccagcgaaccatttgag

SEQ ID NO: 27 - DNA, 51 bp, Artificial sequence
  Synthetic primer used to amplify kanamycin resistance gene
aatgaagttttaaatcaatctaaagtatatagagtcgatacaaattcctcg

SEQ ID NO: 28 - DNA, 7100 bp, Artificial sequence
  Synthetic plasmid pMU166
aattgacaaagttttctatttgtgttaacattgtttatataatagtgaacagtgttaagattaaatgtgaggtgtttgtatggatattaatgattataaagagaagggactttatttattaagtagtatggatgattttattaaaattaatgatttgtttatgggtaaagttgtttctcctggctatgttgcttcggtttttggtgtttccaggtctactgttacacaatggattcaaagacgtaaaattagagcttttaagtataaaggtaaggaaggtgactatatggttatacctattgctgatattattgattacaaaagattgagtaataatgattttatttatgataagttagtgaggtgatttattttatgtttgacgatagctatgttgttaatgagtgttcgtctaatgttagtgaaaatgatagagatttttgtagtttggttggtcgttttatgattattaatggtatagataagttggttattaagattaatagaaaatttaataggaaatctttaagtttagattttagtgttgatttattcccttctatcaaagtttctgaattagttttttttgatgagtttaacaaaacgtgtggtttttatttttcttttaattcttttacaatttttaaggcttttagagatgttcataatcataataaaatatcattttattttgcataatttcgggtctgggccgcagaccaggcccagtgctaacaatattaatttttaatgttaggaattgtttaattcttaattgtgtttttaaaggtagaataattacccattcgccctttagccaacaaaaattaaggaggtataaacatggataaaatggatttgattcttcaagatgaaagactgggtgagatatttaaagatatagatttaacagataatgaaaagagatatcttaaatggttatggaaatgggattatgaaacacgtgatacttttgtatcaatttttttgaagctaaaaaatggtggaaaatgatttttttcttatcttgatatattagaaaaaagcgtactcacgaagtaagaatttgtaaaaaaagaaggggggatttttttggatgagagtttgtacaagcagattttaagtaatattattattactcgtgattattgtaaaaatgttttagataatataaagttcaatgaaaaaataattgattattatgttatgttacaaaatgatgtttttattgattttactaataaaataaattcaataagggattgtaataaatattggtatttggatgtttataaaaagcagaaaataaaggattttaaaaagactaatttgtgtaaagataagttctgtaataattgtaagaaagttaaacaggcttcaagaatgcaaaaatatattcctgaattacagaaatacaaagatggcttatatcattttatatttactgttgaaaatgtgccaggtagtgaattaagagatactattgataggttgtttaagtcttttaagtcatttacaaggtatttaagtggtaatcttaaaataaaaggtgttaattttgataaatggggttataaaggctgtgtaaggtctttagaggtaacttatagtatgattgataatcatattatgtatcatccacacttgcatgttgcgatgatattagatcctttttacgatggttttaatgttgaaaggatgcatataattaataagtttagttatagctatggtgttttaaaaaggttgtttactgatgatgaattattaattcaaaaaatttggtatttattgtttaataatattgaggttaacatggccaatataaataatttagaggatggttattcttgtttagttaataagtttagtgattatgattatgcggagctgtttaagtatatttgtaaaaatactgatgaacaaggtttacttatgacttatgatatttttaaagatttatattttgcattacataatgttcatcagatacaaggctatggttgtttatataatataagagatgatactcaattagatttaaaggttgatgacatttataatgatttgattgatttattacaagttacagaaaatcctatacagtctatggaaactgtacaggatttattaaaggatactgaatatacaataataagccgtaagcgtatatttaagtatctaacacaattatatcataaggattgatatttataccgtctgtcggactcatgcggagggggacttgagggggtctcccctcgcattgtacgacagacggtattattattatacaaattttttttatgtaattttttttgtgtaatttttttatacaaataatatttcaattcgagctcggtacccggggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcagatctgatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggacccatcgataagctagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccacttttcgagatctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatagagtcgatacaaattcctcgtaggcgctcgggacccctatctagcgaacttttagaaaagatataaaacatcagagtatggacagttgcggatgtacttcagaaaagattagatgtctaaaaagctttttagacatctaaatctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttgaaagttggcgtataacatagtatcgacggagccgattttgaaaccacaattatgatagaatttacaagctataaggttattgtcctgggtttcaagcattagtccatgcaagtttttatgctttgcccattctatagatatattgataagcgcgctgcctatgccttgccccctgaaatccttacatacggcgatatcttctatataaaagatatattatcttatcagtattgtcaatatattcaaggcaatctgcctcctcatcctcttcatcctcttcgtcttggtagctttttaaatatggcgcttcatagagtaattctgtaaaggtccaattctcgttttcatacctcggtataatcttacctatcacctcaaatggttcgctgggttaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtg

SEQ ID NO: 29 - DNA, 23 bp, Artificial sequence
  Synthetic consensus sequence
ttttttcttatcttgatatatat

SEQ ID NO: 30 - DNA, 23 bp, Artificial sequence
  Synthetic plasmid pB6A orgin of replication
ttttttcttatcttgatatatta

SEQ ID NO: 31 - DNA, 24 bp, Artificial sequence
  Synthetic plasmid pAO1 origin of replication
ttttttcttatcttgatcaagtgt

SEQ ID NO: 32 - DNA, 24 bp, Artificial sequence
  Synthetic plasmid pC194 origin of replication
ttctttcttatcttgataataacg

SEQ ID NO: 33 - DNA, 24 bp, Artificial sequence
  Synthetic plasmid pNB2 origin of replication
ttttctcttattctgttttaatac

SEQ ID NO: 34 - DNA, 23 bp, Artificial sequence
  Synthetic plasmid pUB110 origin of replication
ttctttcttatcttgatacatat

SEQ ID NO: 35 - DNA, 24 bp, Artificial sequence
  Synthetic plasmid pBC1 origin of replication
ttttttcttatcttgataatatat

SEQ ID NO: 36 - DNA, 24 bp, Artificial sequence
  Synthetic plasmid pBAA1 origin of replication
tcttttcttatcttgatagtatat

SEQ ID NO: 37 - DNA, 23 bp, Artificial sequence
  Synthetic plasmid pBAS2 origin of replication
tttattcttatctatgtatatat

SEQ ID NO: 38 - DNA, 24 bp, Artificial sequence
  Synthetic plasmid pLS11 origin of replication
ttttttcttatcttgatactatat

SEQ ID NO: 39 - DNA, 9801 bp, Artificlal sequence
  Synthetic pMU675 plasmid
aattgacaaagttttctatttgtgttaacattgtttatataatagtgaacagtgttaagattaaatgtgaggtgtttgtatggatattaatgattataaagagaagggactttatttattaagtagtatggatgattttattaaaattaatgatttgtttatgggtaaagttgtttctcctggctatgttgcttcggtttttggtgtttccaggtctactgttacacaatggattcaaagacgtaaaattagagcttttaagtataaaggtaaggaaggtgactatatggttatacctattgctgatattattgattacaaaagattgagtaataatgattttatttatgataagttagtgaggtgatttattttatgtttgacgatagctatgttgttaatgagtgttcgtctaatgttagtgaaaatgatagagatttttgtagtttggttggtcgttttatgattattaatggtatagataagttggttattaagattaatagaaaatttaataggaaatctttaagtttagattttagtgttgatttattcccttctatcaaagtttctgaattagttttttttgatgagtttaacaaaacgtgtggtttttatttttcttttaattcttttacaatttttaaggcttttagagatgttcataatcataataaaatatcattttattttgcataatttcgggtctgggccgcagaccaggcccagtgctaacaatattaatttttaatgttaggaattgtttaattcttaattgtgtttttaaaggtagaataattacccattcgccctttagccaacaaaaattaaggaggtataaacatggataaaatggatttgattcttcaagatgaaagactgggtgagatatttaaagatatagatttaacagataatgaaaagagatatcttaaatggttatggaaatgggattatgaaacacgtgatacttttgtatcaatttttttgaagctaaaaaatggtggaaaatgatttttttcttatcttgatatattagaaaaaagcgtactcacgaagtaagaatttgtaaaaaaagaaggggggatttttttggatgagagtttgtacaagcagattttaagtaatattattattactcgtgattattgtaaaaatgttttagataatataaagttcaatgaaaaaataattgattattatgttatgttacaaaatgatgtttttattgattttactaataaaataaattcaataagggattgtaataaatattggtatttggatgtttataaaaagcagaaaataaaggattttaaaaagactaatttgtgtaaagataagttctgtaataattgtaagaaagttaaacaggcttcaagaatgcaaaaatatattcctgaattacagaaatacaaagatggcttatatcattttatatttactgttgaaaatgtgccaggtagtgaattaagagatactattgataggttgtttaagtcttttaagtcatttacaaggtatttaagtggtaatcttaaaataaaaggtgttaattttgataaatggggttataaaggctgtgtaaggtctttagaggtaacttatagtatgattgataatcatattatgtatcatccacacttgcatgttgcgatgatattagatcctttttacgatggttttaatgttgaaaggatgcatataattaataagtttagttatagctatggtgttttaaaaaggttgtttactgatgatgaattattaattcaaaaaatttggtatttattgtttaataatattgaggttaacatggccaatataaataatttagaggatggttattcttgtttagttaataagtttagtgattatgattatgcggagctgtttaagtatatttgtaaaaatactgatgaacaaggtttacttatgacttatgatatttttaaagatttatattttgcattacataatgttcatcagatacaaggctatggttgtttatataatataagagatgatactcaattagatttaaaggttgatgacatttataatgatttgattgatttattacaagttacagaaaatcctatacagtctatggaaactgtacaggatttattaaaggatactgaatatacaataataagccgtaagcgtatatttaagtatctaacacaattatatcataaggattgatatttataccgtctgtcggactcatgcggagggggacttgagggggtctcccctcgcattgtacgacagacggtattattattatacaaattttttttatgtaattttttttgtgtaatttttttatacaaataatatttcaattcgagctcggtacccggggatcctctagagtcgacctgcaggcatgcacgatacaaattcctcgtaggcgctcgggacccctatctagcgaacttttagaaaagatataaaacatcagagtatggacagttgcggatgtacttcagaaaagattagatgtctaaaaagctttttagacatctaaatctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttgaaagttggcgtataacatagtatcgacggagccgattttgaaaccacaattatgatagaatttacaagctataaggttattgtcctgggtttcaagcattagtccatgcaagtttttatgctttgcccattctatagatatattgataagcgcgctgcctatgccttgccccctgaaatccttacatacggcgatatcttctatataaaagatatattatcttatcagtattgtcaatatattcaaggcaatctgcctcctcatcctcttcatcctcttcgtcttggtagctttttaaatatggcgcttcatagagtaattctgtaaaggtccaattctcgttttcatacctcggtataatcttacctatcacctcaaatggttcgctgggtttgagtcgtgactaagaacgtcaaagtaattaacaatacagctatttttctcatgcttttacccctttcataaaatttaattttatcgttatcataaaaaattatagacgttatattgcttgccgggatatagtgctgggcattcgttggtgcaaaatgttcggagtaaggtggatattgatttgcatgttgatctattgcattgaaatgattagttatccgtaaatattaattaatcatatcataaattaattatatcataattgttttgacgaatgaaggtttttggataaattatcaagtaaaggaacgctaaaaattttggcgtaaaatatcaaaatgaccacttgaattaatatggtaaagtagatataatattttggtaaacatgccttcagcaaggttagattagctgtttccgtataaattaaccgtatggtaaaacggcagtcagaaaaataagtcataagattccgttatgaaaatatacttcggtagttaataataagagatatgaggtaagagatacaagataagagatataaggtacgaatgtataagatggtgcttttaggcacactaaataaaaaacaaataaacgaaaattttaaggaggacgaaagatgttttcggataatttgatacatgcaataaaattcaaaaataatcccacggttgtcggtttggatccaagaattgaaagcattccagaattcataaagaaagcggcctttaataagtacgggaacaatacaaaaggaatatctgaagcgatgtataattttaataaaggcattattgatgctgtatttgatgtagtaccagcggtaaagattcaaattgccttttacgaagtttatggagcagatggaatagaagctttttataaaactgctgaatatgccaaagaaaaagggcttatagttatagcagatgtaaaaagaggtgatatagcagacgtagcagagatgtattcgaaagcatatttgcagaatccatctattgacgcaattacaatcaatccatacatgggagaagataccatgacaccatatatacatgacgtaatagaatacgataaaggactgtttattcttgtgaaaacttccaatgttggttctggtacaattcaaaatttaaaaactatgaatggcactgtgtatgaaaatgtggcatacatggttgataagatttcaaaactggccaaaggcagtttaggatatagttctataggtgcagttgttggagctacgtataaagaggaggccaaaatactgagaaaaataatgccatctgctatctttttggtgcctggatatggagcacagggtgctactgcagaagacgtcattaattgttttgacgaaaacaacttaggtgctatagttaactcatcgagaaaagttatctttgcttataaaagtcaatactggaaagatgtttattctgaatatgagtatgctcaagctgcacgtgctgaagttcttctgatgatggggatgattaataatgcgtttttaaaaagaagatatgttgcgtgttaaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggccttttagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcagatctgatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggacccatcgataagctagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccacttttcgagatctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtg

SEQ ID NO: 40 - DNA, 7633 bp, Artificial Sequence
  Synthetic pMU362 plasmid
aattgacaaagttttctatttgtgttaacattgtttatataatagtgaacagtgttaagattaaatgtgaggtgtttgtatggatattaatgattataaagagaagggactttatttattaagtagtatggatgattttattaaaattaatgatttgtttatgggtaaagttgtttctcctggctatgttgcttcggtttttggtgtttccaggtctactgttacacaatggattcaaagacgtaaaattagagcttttaagtataaaggtaaggaaggtgactatatggttatacctattgctgatattattgattacaaaagattgagtaataatgattttatttatgataagttagtgaggtgatttattttatgtttgacgatagctatgttgttaatgagtgttcgtctaatgttagtgaaaatgatagagatttttgtagtttggttggtcgttttatgattattaatggtatagataagttggttattaagattaatagaaaatttaataggaaatctttaagtttagattttagtgttgatttattcccttctatcaaagtttctgaattagttttttttgatgagtttaacaaaacgtgtggtttttatttttcttttaattcttttacaatttttaaggcttttagagatgttcataatcataataaaatatcattttattttgcataatttcgggtctgggccgcagaccaggcccagtgctaacaatattaatttttaatgttaggaattgtttaattcttaattgtgtttttaaaggtagaataattacccattcgccctttagccaacaaaaattaaggaggtataaacatggataaaatggatttgattcttcaagatgaaagactgggtgagatatttaaagatatagatttaacagataatgaaaagagatatcttaaatggttatggaaatgggattatgaaacacgtgatacttttgtatcaatttttttgaagctaaaaaatggtggaaaatgatttttttcttatcttgatatattagaaaaaagcgtactcacgaagtaagaatttgtaaaaaaagaaggggggatttttttggatgagagtttgtacaagcagattttaagtaatattattattactcgtgattattgtaaaaatgttttagataatataaagttcaatgaaaaaataattgattattatgttatgttacaaaatgatgtttttattgattttactaataaaataaattcaataagggattgtaataaatattggtatttggatgtttataaaaagcagaaaataaaggattttaaaaagactaatttgtgtaaagataagttctgtaataattgtaagaaagttaaacaggcttcaagaatgcaaaaatatattcctgaattacagaaatacaaagatggcttatatcattttatatttactgttgaaaatgtgccaggtagtgaattaagagatactattgataggttgtttaagtcttttaagtcatttacaaggtatttaagtggtaatcttaaaataaaaggtgttaattttgataaatggggttataaaggctgtgtaaggtctttagaggtaacttatagtatgattgataatcatattatgtatcatccacacttgcatgttgcgatgatattagatcctttttacgatggttttaatgttgaaaggatgcatataattaataagtttagttatagctatggtgttttaaaaaggttgtttactgatgatgaattattaattcaaaaaatttggtatttattgtttaataatattgaggttaacatggccaatataaataatttagaggatggttattcttgtttagttaataagtttagtgattatgattatgcggagctgtttaagtatatttgtaaaaatactgatgaacaaggtttacttatgacttatgatatttttaaagatttatattttgcattacataatgttcatcagatacaaggctatggttgtttatataatataagagatgatactcaattagatttaaaggttgatgacatttataatgatttgattgatttattacaagttacagaaaatcctatacagtctatggaaactgtacaggatttattaaaggatactgaatatacaataataagccgtaagcgtatatttaagtatctaacacaattatatcataaggattgatatttataccgtctgtcggactcatgcggagggggacttgagggggtctcccctcgcattgtacgacagacggtattattattatacaaattttttttatgtaattttttttgtgtaatttttttatacaaataatatttcaattcgagctcggtacccgggatatggatccagcttccaaggagctaaagaggtccctagcgcctacggggaatttgtatcgataaggggtacaaattcccactaagcgctcggcggggatcgatcccgggtacgtacccggcagtttttctttttcggcaagtgttcaagaagttattaagtcgggagtgcagtcgaagtgggcaagttgaaaaattcacaaaaatgtggtataatatctttgttcattagagcgataaacttgaatttgagagggaacttagatggtatttgaaaaaattgataaaaatagttggaacagaaaagagtattttgaccactactttgcaagtgtaccttgtacatacagcatgaccgttaaagtggatatcacacaaataaaggaaaagggaatgaaactatatcctgcaatgctttattatattgcaatgattgtaaaccgccattcagagtttaggacggcaatcaatcaagatggtgaattggggatatatgatgagatgataccaagctatacaatatttcacaatgatactgaaacattttccagcctttggactgagtgtaagtctgactttaaatcatttttagcagattatgaaagtgatacgcaacggtatggaaacaatcatagaatggaaggaaagccaaatgctccggaaaacatttttaatgtatctatgataccgtggtcaaccttcgatggctttaatctgaatttgcagaaaggatatgattatttgattcctatttttactatggggaaatattataaagaagataacaaaattatacttcctttggcaattcaagttcatcacgcagtatgtgacggatttcacatttgccgttttgtaaacgaattgcaggaattgataaatagttaacttcaggtttgtctgtaactaaaaacaagtatttaagcaaaaacatcgtagaaatacggtgttttttgttaccctaaaatctacaattttatacataaccacaggttagtacaaagaccttgtgtttctttttgaaaggcttaaaacaaggatttttccttgatttaagccccgaaaagcaacacaaccaaggttttagtatcaatctgtggtttttatattttcagagaaaaggagaacaagaaaaaatgaaactaaatgaaaacgaaatgaatttcagcgtacctcttgaaatcatcaaggcaagtgaaatcgagccgaaagaagtaaagtggctgtggtatccgtatattccgctgcagatatgcatgcaagcttggctgcaggtcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgtggtttcaaaatcggctccgtcgatactatgttatacgccaactttcaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatttagatgtctaaaaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtg